(19) United States
(12) Patent Application Publication
(10) Pub. No.: US 2023/0040597 A1
(43) Pub. Date: Feb. 9, 2023

(54) USE OF NICOLSAMIDE FORMULATIONS FOR ANTIVIRAL THERAPY

(71) Applicant: YourChoice Therapeutics, Inc., Berkeley, CA (US)

(72) Inventors: Nadja MANNOWETZ, Berkeley, CA (US); Akash BAKSHI, Berkeley, CA (US); Andrew BARTYNSKI, Berkeley, CA (US)

(21) Appl. No.: 17/877,602

(22) Filed: Jul. 29, 2022

Related U.S. Application Data
(63) Continuation of application No. PCT/US2021/018844, filed on Feb. 19, 2021.
(60) Provisional application No. 62/980,105, filed on Feb. 21, 2020.
(60) Provisional application No. 63/020,519, filed on May 5, 2020.
(60) Provisional application No. 63/049,086, filed on Jul. 7, 2020.

(51) Int. Cl. A61K 31/167; A61K 9/06; A61K 47/12; A61K 47/02; A61K 47/10

(52) U.S. Cl. CPC A61K 31/167; A61K 9/06; A61K 47/12; A61K 47/02; A61K 47/10

(57) ABSTRACT

Disclosed are niclosamide formulations for use as antiviral therapy. The formulations disclosed herein may be used for treating a sexually-transmitted virus or a respiratory virus (e.g., coronavirus).

### RELATED APPLICATIONS

This application is a continuation of International Application No. PCT/US2021/018844, filed Feb. 19, 2021, which claims priority to and the benefit of U.S. Provisional Patent Application No. 62/980,105, filed on Feb. 21, 2020, U.S. Provisional Patent Application No. 63/020,519, filed on May 5, 2020, and U.S. Provisional Patent Application No. 63/049,086, filed on Jul. 7, 2020, the entire contents of which are incorporated herein by reference.

### FIELD OF THE DISCLOSURE

The present invention is directed to formulations of niclosamide for use in gels and creams and methods of using the same, in methods of contraception and methods of treating and preventing infection.

### BACKGROUND

Present contraceptive methods depend on hormonal control mechanisms or on physically disrupting contact between sperm cells and an egg using a barrier method. Women are more often the subject of these methods and, consequently, experience the negative side effects and secondary medical complications of systemic medication or implantation in the case of intrauterine devices. There is a long-felt and unmet need for non-hormonal and unisex, contraceptive compositions and methods that are effective, safe and easily applicable.

The increasing prevalence of sexually transmitted diseases (STD), like acquired immune deficiency syndrome (AIDS), is a serious public health problem worldwide. Conventional contraceptive formulations and devices are ineffective in preventing transmission of STDs. Apart from a contraceptive use, barrier methods for preventing transmission of fluids between individuals are not effective at preventing transmission of STDs.

A need in the art exists, both within the context of contraception and in a larger context of sexual contact, for a safe, efficacious, unisex, non-hormonal, non-barrier agent that can treat or prevent STD transmission.

### SUMMARY

The disclosure provides formulations of niclosamide, and use of the same, for treating or preventing infection, either alone or in combination with promoting contraception by reducing or inhibiting sperm motility. The formulations of the disclosure are non-hormonal, unisex with respect to administration and transient in terms of clearance from the body of a subject.

Provided herein, is a method of treating or preventing an infection by a virus in a subject having or at risk for a viral infection and/or inhibiting or preventing viral replication of a virus in a subject having or at risk for a viral infection, comprising administering to a subject an effective amount of a formulation comprising an amount of niclosamide and one or more of a humectant, a lubricant, a solvent, an osmolality modulator, a pH modulator, a viscosity modulator and a preservative, wherein the formulation inhibits viral replication of the virus, optionally thereby treating or preventing a viral infection

In some embodiments of the method of the disclosure, the virus is a Herpes-Simplex virus (HSV), Human immunodeficiency virus (HIV), Coronavirus, Severe acute respiratory syndrome coronavirus (SARS-CoV-1), Severe acute respiratory syndrome coronavirus (SARS-CoV-2), Human coronavirus-Subtype OC43 (HCoV-OC43), Middle eastern respiratory syndrome corona virus (MERS-CoV), Vesicular stomatitis virus—Zaire Ebola virus (VZVEBOV), Dengue virus (DENY), Powassan virus (POWV), Enterovirus (EV), Influenza A virus (FluAV), Human metapneumovirus (HMPV), Rabies virus (RABV), Rift valley fever virus (RVFV), Zika virus (ZIKV), West-nile virus (WNV), Yellow fever virus (YFV), Sindbis virus (SINV), Junin virus (JUNV), Lassa mammarenavirus (LASV), or Lymphocytic choriomeningitis virus (LCMV). In some embodiments of the method of the disclosure, the virus is a Coronavirus. In some embodiments of the method of the disclosure, the virus is a SARS-CoV-2.

In some embodiments of the method of the disclosure, the administering step results in a concentration of niclosamide at about 150 mM in the subject. In some embodiments of the method of the disclosure, the systemic concentration of niclosamide is concentration of niclosamide in blood, serum or plasma of the subject.

In some embodiments of the method of the disclosure, the administration step results in a concentration of niclosamide at a site of administration of the formulation in the subject, of about 100 nM to about 1 μM. In some embodiments of the method of the disclosure, the administration step results in a concentration of niclosamide at a site of administration of the formulation in the subject, of about 100 nM to about 500 nM. In some embodiments of the method of the disclosure, the administration step results in a concentration of niclosamide at a site of administration of the formulation in the subject, of about 100 nM to about 250 nM. In some embodiments of the method of the disclosure, the administration step results in a concentration of niclosamide at a site of administration of the formulation in the subject, of about 100 nM to about 150 nM. In some embodiments of the method of the disclosure, the administration step results in a concentration of niclosamide at a site of administration of the formulation in the subject, at about 150 nM.

In some embodiments of the method of the disclosure, the formulation is administered locally. In some embodiments of the method of the disclosure, the formulation is administered topically, intrarectally, intramuscularly or intravaginally. In some embodiments of the method of the disclosure, the subject is human.

The formulations of the disclosure treat or prevent an infection and/or reduce or prevent transmission of an infection. In some embodiments of the disclosure, the formulation comprises niclosamide and one or more excipients. In some embodiments, the one or more excipients comprise one or more of a humectant, a lubricant, a solvent, an osmolality modulator, a pH modulator, a viscosity modulator and a preservative. In some embodiments of the disclosure, the formulation comprises the amount of niclosamide in a powder form. In some embodiments of the disclosure, the formulation comprises the amount of niclosamide in a liquid form. In some embodiments of the disclosure, the formulation comprises a capsule comprising the amount of niclosamide. In some embodiments of the disclosure, the capsule further comprises the one or more of a humectant, a lubricant, a solvent, an osmolality modulator, a pH modulator, a viscosity modulator and a preservative. In some embodiments of the disclosure, the formulation comprises a suspension comprising the amount of niclosamide. In some embodiments, including those in which the formulation is suitable for use in the treatment of an infection, the formulation further comprises a second therapeutic agent. In some embodiments, the second therapeutic agent comprises one or more of Obatoclax, Emetine, Homoharringtonin, Brequinar and Suramin. In some embodiments, the second therapeutic agent comprises Emetine. In some embodiments, the second therapeutic agent comprises Homoharringtonin.

In some embodiments of the formulations of the disclosure, the formulation has a viscosity of between about 180 cPs and about 58,000 cPs. In some embodiments of the formulations of the disclosure, the formulation has a viscosity of between about 180 cPs and about 16,000 cPs. In some embodiments of the formulations of the disclosure, the formulation has a viscosity of between about 700 cPs and about 58,000 cPs. In some embodiments of the formulations of the disclosure, the viscosity modulator is hydroxyethyl cellulose (HEC), or carbopol, or polycarbophil, or pemulen. In some embodiments of the formulations of the disclosure, the formulation has a viscosity of between about 10,000 cPs and about 18,000 cPs. In some embodiments of the formulations of the disclosure, the viscosity modulator is carbopol or polycarbophil. In some embodiments of the formulations of the disclosure, the formulation has a viscosity of 11,000 cPs. In some embodiments of the formulations of the disclosure, the viscosity modulator is carbopol.

In some embodiments of the formulations of the disclosure, the humectant, the lubricant or the solvent comprise a synthetic polymer or water. In some embodiments of the formulations of the disclosure, the humectant, the lubricant or the solvent comprise a synthetic polymer. In some embodiments, the humectant, the lubricant or the solvent comprise at least two monomers of polyethylene glycol (PEG). In some embodiments of the disclosure, including those wherein the formulation or the niclosamide is provided as a power, a liquid or a suspension, the formulation is provided in a unit dosage form. In some embodiments, the unit dosage form comprises a single dose of the formulation and a single dose comprises an amount of niclosamide effective to reduce, partially or completely, a risk of infection by one or more of a virus, a bacterium or an archaea. In some embodiments, the unit dosage form comprises a single dose of the formulation and a single dose comprises an amount of niclosamide effective to reduce, partially or completely, a risk of infection by a virus. In some embodiments, the unit dosage form comprises a single dose of the formulation and a single dose comprises an amount of niclosamide effective to reduce, partially or completely, a risk of infection by a bacterium. In some embodiments, the unit dosage form comprises a single dose of the formulation and a single dose comprises an amount of niclosamide effective to reduce, partially or completely, a risk of infection by an archaea.

In some embodiments of the disclosure, including those wherein the formulation or the niclosamide is provided as a power, a liquid or a suspension, the formulation is provided in a unit dosage form. In some embodiments, the unit dosage form comprises at least two doses of the formulation and a single dose comprises an amount of niclosamide effective to reduce, partially or completely, a risk of infection by one or more of a virus, a bacterium or an archaea. In some embodiments, the unit dosage form comprises at least two doses of the formulation and a single dose comprises an amount of niclosamide effective to reduce, partially or completely, a risk of infection by a virus. In some embodiments, the unit dosage form comprises at least two doses of the formulation and a single dose comprises an amount of niclosamide effective to reduce, partially or completely, a risk of infection by a bacterium. In some embodiments, the unit dosage form comprises at least two doses of the formulation and wherein a single dose comprises an amount of niclosamide effective to reduce, partially or completely, a risk of infection by an archaea.

In some embodiments of the disclosure, including those wherein the formulation or the niclosamide is provided as a power, a liquid or a suspension, the virus has a reproduction number (R0) of between 0.9 and 18. In some embodiments, the virus has a R0 of between 1.5 and 3.5. In some embodiments, the virus has a R0 of between 2 and 5.

In some embodiments of the disclosure, including those wherein the formulation or the niclosamide is provided as a power, a liquid or a suspension, the virus is an RNA virus. In some embodiments, the RNA virus is a positive-strand RNA virus. In some embodiments, the positive-strand RNA virus belongs to the family of Picornaviridae, Astroviridae, Caliciviridae, Hepeviridae Flaviviridae, Togaviridae, Arteriviridae, or Coronaviridae. In some embodiments, the positive-strand RNA virus belongs to the family of Coronaviridae. In some embodiments, the Coronaviridae virus is Bat coronavirus CDPHE15, Bat coronavirus HKU10, Rhinolophus ferrumequinum alphacoronavirus HuB-2013, Human coronavirus 229E, Lucheng Rn rat coronavirus, Ferret coronavirus, Mink coronavirus 1, Miniopterus bat coronavirus 1, Miniopterus bat coronavirus HKU8, Myotis ricketti alphacoronavirus Sax-2011, Nyctalus velutinus alphacoronavirus SC-2013, Porcine epidemic diarrhea virus, Scotophilus bat coronavirus 512, Rhinolophus bat coronavirus HKU2, Human coronavirus NL63, NL63-related bat coronavirus strain BtKYNL63-9b, Human coronavirus OC43, China Rattus coronavirus HKU24, Human coronavirus HKU1, Murine coronavirus—type species, Bat Hp-betacoronavirus Zhejiang2013, Hedgehog coronavirus 1, Middle East respiratory syndrome-related coronavirus (MERS-CoV), Pipistrellus bat coronavirus HKU5, Tylonycteris bat coronavirus HKU4, Rousettus bat coronavirus GCCDC1, Rousettus bat coronavirus HKU9, Severe acute respiratory syndrome-related coronavirus, Wigeon coronavirus HKU20, Bulbul coronavirus HKU11—type species, Porcine coronavirus HKU15, Munia coronavirus HKU13, White-eye coronavirus HKU16, Night heron coronavirus HKU19, Common moorhen coronavirus HKU21, Beluga whale coronavirus SW1 or Avian coronavirus—type species. In some embodiments, the e Coronaviridae virus is Middle East respiratory syndrome-related coronavirus (MERS-CoV), Severe acute respiratory syndrome coronavirus (SARS-CoV) or Severe acute respiratory syndrome coronavirus 2 (SARS-CoV-2). In some embodiments, the Coronaviridae virus is Severe acute respiratory syndrome coronavirus 2 (SARS-CoV-2).

In some embodiments of the disclosure, including those wherein the formulation or the niclosamide is provided as a power, a liquid or a suspension, the RNA virus is a negative-strand RNA virus. In some embodiments, the negative-strand RNA virus belongs to the family of Orthomyxoviridae, Paramyxoviridae, Rhabdoviridiae, Filoviruses, or Arenaviruses. In some embodiments, the negative-strand virus is Influenza virus, Sendai virus, Human parainfluenza virus 1 (hPIV1), Simian virus 5 (SV5, PIV5), Mumps virus, Newcastle disease virus (NDV), Measles virus, Rinderpest virus, Respiratory syncytial virus (RSV), Vesicular stomatitis virus (VSV), Rabies virus, Ebola virus, Marburg virus, Lymphocytic choriomeningitis virus (LCMV), Junin virus, or Lassa fever virus.

In some embodiments of the disclosure, including those wherein the formulation or the niclosamide is provided as a power, a liquid or a suspension, the virus is a retrovirus. In some embodiments, the retrovirus is a Lentivirus, Alpharetrovirus, Betaretrovirus, Deltaretrovirus, Gammaretrovirus or Epsilonretrovirus. In some embodiments, the retrovirus is a Simian immunodeficiency virus (SIV), Human immunodeficiency virus-1 (HIV-1), HIV-2, Feline immunodeficiency virus (FIV), Equine infectious anemia virus (EIAV), Mouse mammary tumor-like virus (MMTV), Mason-Pfizer monkey virus (MPMV), Respiratory syncytial virus RSV, bovine leukemia virus (BLV), Human T-cell leukemia virus-1 (HTLV-1), HTLV-2, Murine leukemia virus (MuLV), Gibbon ape leukemia virus (GALV).

In some embodiments of the disclosure, including those wherein the formulation or the niclosamide is provided as a power, a liquid or a suspension, the virus is a DNA virus. In some embodiments, the DNA virus is Adenovirus, infectious canine hepatitis virus, Papillomavirus, polyomaviridae, simian vacuolating virus, Parvovirus B19, canine parvovirus, Herpes simplex virus, varicella-zoster virus, cytomegalovirus, Epstein-Barr virus, Smallpox virus, cow pox virus, sheep pox virus, monkey pox virus, vaccinia virus, Torque teno virus.

In some embodiments of the disclosure, including those wherein the formulation or the niclosamide is provided as a power, a liquid or a suspension, a food or a beverage comprises the formulation. In some embodiments, the combination of the food or the beverage and the formulation forms a suspension. In some embodiments, the combination of the food or the beverage and the formulation forms a liquid. In some embodiments, the formulation dissolves in the food or the beverage. In some embodiments, the food or the beverage comprises one or more of a dietary supplement, a meal replacement, an electrolyte, a protein, a sugar and a pharmaceutical carrier. In some embodiments, the food or the beverage comprises a pharmaceutical carrier and wherein one or more of an intravenous tube, a feeding tube or a central line comprise the formulation. In some embodiments, the pharmaceutical carrier or the formulation comprises one or more of a nanoparticle or a polymer. In some embodiments, the food or the beverage comprises a pediatric formula, a diabetic formula or a senior formula. In some embodiments, the food or the beverage comprises caffeine. In some embodiments, the food or the beverage comprises alcohol.

The formulations of the disclosure treat or prevent an infection and/or reduce or prevent transmission of an infection. In some embodiments of the disclosure, the infection may be a sexually-transmitted pathogen (e.g. a virus or bacteria) that induces the onset of a disease or condition referred to as a sexually-transmitted disease (STD). In some embodiments of the disclosure, the infection is transduced by a virus. In some embodiments of the disclosure, the infection is transduced by a bacterium. In some embodiments of the disclosure, the infection may be transmitted by physical contact or proximity, including but not limited to communication through air, fluid, droplet (e.g. breath, sneeze, cough), and/or direct physical transmission (by touch).

In some embodiments of the disclosure, the formulation is used as both a contraceptive and an anti-viral or anti-bacterial agent. In some embodiments, niclosamide is provided as a contraceptive formulation (which includes any formulation of the disclosure) and an anti-viral or an anti-bacterial formulation (which includes any formulation of the disclosure). Whereas the contraceptive formulation are provided preferably in the form of a gel or a cream, when used in combination with niclosamide in an anti-viral or anti-bacterial formulation, the anti-viral or anti-bacterial formulation may be provided in any form (including, but not limited to, a solid, a liquid or a semi-solid). When a contraceptive formulation and an anti-viral or anti-bacterial formulation are provided, the contraceptive formulation may be a gel or a cream administered by a local route (e.g. topically or intra-cavitally to a component of the male or female reproductive system) and the anti-viral or anti-bacterial formulation may be of any form (e.g. a pill, tablet, capsule, suspension, and liquid) administered by a systemic route (e.g. oral or intravenous). When a contraceptive formulation and an anti-viral or anti-bacterial formulation are provided, the contraceptive formulation may be a gel or a cream administered by a local route (e.g. topically or intra-cavitally to a component of the male or female reproductive system) and at a concentration of between 4 mM and 100 mM and the anti-viral or anti-bacterial formulation may be of any form (e.g. a pill, tablet, capsule, suspension, and liquid) administered by a systemic route (e.g. oral or intravenous) and at a concentration of between 0.01 μM and 30 μM. When a contraceptive formulation and an anti-viral or anti-bacterial formulation are provided, the contraceptive formulation may be a gel or a cream administered by a local route (e.g. topically or intra-cavitally to a component of the male or female reproductive system) and at a concentration of between 4 mM and 100 mM and the anti-viral or anti-bacterial formulation may be of any form (e.g. a pill, tablet, capsule, suspension, and liquid) administered by a systemic route (e.g. oral or intravenous) and at a dose of between 0.5 and 2 grams per day (by oral tablet having 500 mg niclosamide).

In some embodiments of the disclosure, the formulation is used only as an anti-viral or anti-bacterial agent. When used an anti-viral or anti-bacterial alone, niclosamide may be formulated as a gel or a cream. When used an anti-viral or anti-bacterial alone, niclosamide may be administered locally. For example, the niclosamide formulation may be administered topically or intra-cavitally to a component of the male or female reproductive system. When used an anti-viral or anti-bacterial alone, niclosamide may be administered locally and at a concentration of between 0.01 μM and 30 μM. When used an anti-viral or anti-bacterial alone, niclosamide may be administered locally, “on demand,” prior to sexual contact. In some embodiments, the anti-viral or anti-bacterial formulation may be administered locally less than 1 minute (min), 2 min, 3 min, 4 min, 5 min, 10 min, 15 min, 20 min, 25 min, 30 min, 35 min, 40 min, 45 min, 50 min, 55 min, 60 min or any number of minutes in between prior to contact. In some embodiments, the anti-viral or anti-bacterial formulation may be administered locally less than 1 hour prior to contact.

The disclosure provides a formulation comprising niclosamide and one or more excipients. In some embodiments, the one or more excipients comprise one or more of a humectant, a lubricant, a solvent, an osmolality modulator, a pH modulator, a viscosity modulator and a preservative.

The disclosure provides a formulation comprising an amount of niclosamide and one or more of a humectant, a lubricant, a solvent, an osmolality modulator, a pH modulator, a viscosity modulator and a preservative.

In some embodiments of the formulations of the disclosure, the humectant, the lubricant or the solvent comprises at least two monomers of polyethylene glycol (PEG), optionally PEG-400, PEG-1000, or PEG-2000. In some embodiments, the humectant, the lubricant or the solvent comprise PEG-400. In some embodiments of the formulations of the disclosure, the formulation comprises an amount of PEG-400 between 0.1% and 75% of the total weight of the formulation, inclusive of the endpoints. In some embodiments of the formulations of the disclosure, the amount of PEG-400 is between 35% and 75% of the total weight of the formulation, inclusive of the endpoints. In some embodiments of the formulations of the disclosure, the amount of PEG-400 is between 35% and 65% of the total weight of the formulation, inclusive of the endpoints. In some embodiments of the formulations of the disclosure, the amount of PEG-400 is about 65% of the total weight of the formulation, inclusive of the endpoints. In some embodiments of the formulations of the disclosure, the amount of PEG-400 is 65% of the total weight of the formulation, inclusive of the endpoints.

In some embodiments, the formulation comprises an amount of the humectant, the lubricant or the solvent of between 0.1% and 65% of the total weight of the formulation, inclusive of the endpoints. In some embodiments, the solvent comprises water and the formulation comprise an amount of the water of between 10% and 90% of the total weight of the formulation, inclusive of the endpoints.

In some embodiments of the formulations of the disclosure, the formulations comprise a semi-solid form. In some embodiments, the semi-solid form comprises one or more of a gel, a hydrogel, a dehydrated gel, a hydrated gel, a paste, an ointment, a cream, and a lotion. In some embodiments, formulations of the disclosure comprise a gel or a cream.

In some embodiments of the formulations of the disclosure, the amount of niclosamide is between 0.1% and 10% of total weight of the formulation, inclusive of the endpoints. In some embodiments, the amount of niclosamide is between 0.1% and 5% of total weight of the formulation, inclusive of the endpoints. In some embodiments, the amount of niclosamide is between 1% and 5% of total weight of the formulation, inclusive of the endpoints. In some embodiments of the formulations of the disclosure, the amount of niclosamide is between 3% and 7% of total weight of the formulation, inclusive of the endpoints. In some embodiments of the formulations of the disclosure, the amount of niclosamide is between 3% and 6% of total weight of the formulation, inclusive of the endpoints. In some embodiments of the formulations of the disclosure, the amount of niclosamide is about 5% of total weight of the formulation. In some embodiments of the formulations of the disclosure, the amount of niclosamide is 5% of total weight of the formulation. In some embodiments, the amount of niclosamide is between 3% and 4% of total weight of the formulation, inclusive of the endpoints. In some embodiments, the amount of niclosamide is about 3.27% of total weight of the formulation. In some embodiments, the amount of niclosamide is 3.27% of total weight of the formulation.

In some embodiments of the formulations of the disclosure, the niclosamide has a concentration of between 4 mM and 500 mM, inclusive of the endpoints. In some embodiments, the niclosamide has a concentration of between 10 mM and 250 mM, inclusive of the endpoints. In some embodiments of the formulations of the disclosure, the niclosamide has a concentration of about 150 mM. In some embodiments of the formulations of the disclosure, the niclosamide has a concentration of 150 mM. In some embodiments, the niclosamide has a concentration of about 100 mM. In some embodiments, the niclosamide has a concentration of 100 mM. In some embodiments of the formulations of the disclosure, the niclosamide formulation may be a gel or a cream administered by a local route (e.g., intramuscularly) to a subject in need thereof to achieve a local concentration of 150 nM.

In some embodiments of the formulations of the disclosure, the humectant, the lubricant or the solvent comprise a synthetic polymer or water. In some embodiments, the humectant, the lubricant or the solvent comprise at least two monomers of polyethylene glycol (PEG). In some embodiments, the humectant, the lubricant or the solvent comprise PEG-400. In some embodiments, the formulation comprises an amount of the humectant, the lubricant or the solvent of between 0.1% and 65% of the total weight of the formulation, inclusive of the endpoints. In some embodiments, the solvent comprises water and the formulation comprise an amount of the water of between 10% and 90% of the total weight of the formulation, inclusive of the endpoints.

In some embodiments of the formulations of the disclosure, the osmolality modulator comprises sodium chloride. In some embodiments, the formulation comprises an amount of the osmolality modulator of less than 5% of the total weight of the formulation. In some embodiments, the formulation comprises an amount of the osmolality modulator of less than 1% of the total weight of the formulation.

In some embodiments of the formulations of the disclosure, the pH modulator comprises one or more of lactic acid, citric acid monohydrate, potassium sodium tartrate, potassium bitartrate and sodium hydroxide. In some embodiments, the formulation comprises an amount of the pH modulator of less than 10% of the total weight of the formulation and wherein the pH modulator comprise lactic acid, potassium sodium tartrate, citric acid monohydrate, potassium bitartrate, and sodium hydroxide. In some embodiments, the formulation comprises an amount of lactic acid of less than 5% of the total weight of the formulation and wherein the pH modulator comprises citric acid monohydrate, potassium sodium tartrate, potassium bitartrate, and sodium hydroxide. In some embodiments, the formulation comprises an amount of the pH modulator of less than 1% of the total weight of the formulation and wherein the pH modulator comprises one or more of potassium sodium tartrate, potassium bitartrate and sodium hydroxide. In some embodiments, the formulation comprises an amount of the pH modulator of less than 0.5% of the total weight of the formulation and wherein the pH modulator comprises one or more of potassium sodium tartrate, potassium bitartrate and sodium hydroxide.

In some embodiments of the formulations of the disclosure, the viscosity modulator is a viscosity enhancer. In some embodiments, the viscosity enhancer comprises one or more of hydroxyethyl cellulose, alginic acid, polycarbophil and carbopol. In some embodiments, the formulation comprises an amount of a viscosity enhancer of between 1% and 10% of the total weight of the formulation, inclusive of the endpoints. In some embodiments, the formulation comprises an amount of a viscosity enhancer of between 1% and 5% of the total weight of the formulation, inclusive of the endpoints. In some embodiments, the formulation comprises an amount of a viscosity enhancer of less than 5% of the total weight of the formulation. In some embodiments, the viscosity enhancer comprises one or more of hydroxyethyl cellulose, alginic acid, polycarbophil and carbopol. In some embodiments, the formulation comprises an amount of a viscosity enhancer of 3% of the total weight of the formulation. In some embodiments, the viscosity enhancer comprises hydroxyethyl cellulose. In some embodiments, the formulation comprises an amount of a viscosity enhancer less than 1% of the total weight of the formulation. In some embodiments, the formulation comprises an amount of a viscosity enhancer of about 0.5% of the total weight of the formulation. In some embodiments, the formulation comprises an amount of a viscosity enhancer of 0.5% of the total weight of the formulation. In some embodiments, the viscosity enhancer comprises carbopol. In some embodiments, the carbopol is carbopol-980.

In some embodiments, the preservative comprises one or more of benzyl alcohol, chlorhexidine gluconate and benzoic acid. In some embodiments, the preservative comprises benzyl alcohol or benzoic acid. In some embodiments, the preservative comprises benzyl alcohol. In some embodiments of the formulations of the disclosure, the formulation comprises an amount of the preservative of less than 1% of the total weight of the formulation. In some embodiments, the formulation comprises an amount of the preservative of about 0.2% of the total weight of the formulation. In some embodiments, the formulation comprises an amount of the preservative of 0.2% of the total weight of the formulation. In some embodiments, the formulation comprises an amount of the preservative of less than 0.2% of the total weight of the formulation. In some embodiments, the preservative comprises benzyl alcohol or benzoic acid. In some embodiments, the formulation comprises an amount of the preservative of less than 0.12% of the total weight of the formulation. In some embodiments, the preservative comprises chlorhexidine gluconate.

In some embodiments of the formulation of the disclosure, when administered as a monotherapy, the formulation does not comprise a dry solid formulation. In some embodiments, the formulation does not comprise one or more of a pill, a powder, a capsule, a tablet or any combination thereof.

In some embodiments of the formulations of the disclosure, the formulation comprises, a) niclosamide at a concentration of 3% to 5% by weight of the formulation; b) a humectant, a lubricant or a solvent, wherein the humectant, the lubricant or the solvent comprises an amount of PEG-400 of 35% to 65% by weight of the formulation and an amount of water of 10% to 60% by weight of the formulation; c) an osmolality modulator, wherein the osmolality modulator comprises an amount of sodium chloride of 0.1% to 1% by weight of the formulation; d) at least one pH modulator, wherein the at least one pH modulator comprises an amount of lactic acid of 1% to 5% by weight of the formulation, an amount of citric acid monohydrate of 1% to 5% by weight of the formulation, an amount of potassium sodium tartrate of 0.1% to 0.5% by weight of the formulation, and an amount of sodium hydroxide of 0.1% to 0.5% by weight of the formulation; e) at least one viscosity modulator, wherein the at least one viscosity modulator comprises an amount of carbopol of 0.1% to 1% by weight of the formulation; and f) at least one preservative, wherein the at least one preservative comprises an amount of benzyl alcohol of 0.1% to 0.5% by weight of the formulation.

In some embodiments of the formulations of the disclosure, the formulation comprises a) niclosamide at a concentration of 100 mM by weight of the formulation; b) a humectant, a lubricant or a solvent, wherein the humectant, the lubricant or the solvent comprises an amount of PEG-400 of between 0.1% and 65% by weight of the formulation and an amount of water of between 10% and 90% by weight of the formulation; c) an osmolality modulator, wherein the osmolality modulator comprise an amount of sodium chloride of less than 1% by weight of the formulation; d) at least one pH modulator, wherein the at least one pH modulator comprises san amount of lactic acid of less than 10% by weight of the formulation, an amount of citric acid monohydrate of less than 5% by weight of the formulation, an amount of potassium bitartrate of less than 0.5% by weight of the formulation, and an amount of sodium hydroxide of less than 1% by weight of the formulation; e) at least one viscosity modulator, wherein the at least one viscosity modulator comprises an amount of hydroxyethyl cellulose of 3% by weight of the formulation, an amount of alginic acid of less than 5% by weight of the formulation, an amount of polycarbophil of less than 5% by weight of the formulation and an amount of carbopol of less than 5% by weight of the formulation; and f) at least one preservative, wherein the at least one preservative comprises an amount of benzyl alcohol of less than 1% by weight of the formulation, an amount of chlorhexidine gluconate of less than 0.12% by weight of the formulation, and an amount of benzoic acid of less than 0.2% by weight of the formulation. In some embodiments of the formulation of the disclosure, when administered as a monotherapy, the formulation does not comprise a dry solid formulation. In some embodiments, the formulation does not comprise one or more of a pill, a powder, a capsule, a tablet or any combination thereof.

The disclosure provides a method of contraception, comprising administering an effective amount of the formulation of the disclosure to the subject, wherein, upon contacting a sperm cell, the formulation inhibits or decreases one or more of sperm motility, sperm viability, and sperm metabolism, thereby decreasing the probability of conception.

In some embodiments of the methods of the disclosure, the formulation inhibits or decreases sperm motility by about 60%, 65%, 70%, 75%, 80%, 85%, 90%, 95%, 100%, or any percentage in between in comparison to a control value. In some embodiments, the control value is a predetermined value. In some embodiments, the control value is an average value of sperm motility measured from healthy sperm cells obtained from one or more individual donors. In some embodiments, the healthy sperm cells have not contacted the formulation. In some embodiments, the control value is determined in vitro.

In some embodiments of the methods of contraception of the disclosure, the formulation contacts a sperm cell for less than 2 minutes. In some embodiments, the formulation is administered to the subject less than 30 minutes prior to intercourse. In some embodiment, the formulation is administered to the subject between 15 and 20 minutes prior to intercourse.

In some embodiments of the methods of contraception of the disclosure, the formulation is administered locally. In some embodiments, the formulation is administered topically. In some embodiments, the subject is male. In some embodiments, the formulation is administered intravaginally. In some embodiments, the subject is female.

The disclosure provides a formulation of the disclosure for use in promoting contraception, comprising administering an effective amount of the formulation to the subject, wherein, upon contacting a sperm cell, the formulation inhibits or decreases one or more of sperm motility, sperm viability, and sperm metabolism, thereby decreasing the probability of conception.

In some embodiments of the formulations of the disclosure for use in promoting contraception, the formulation inhibits or decreases sperm motility by about 60%, 65%, 70%, 75%, 80%, 85%, 90%, 95%, 100%, or any percentage in between in comparison to a control value. In some embodiments, the control value is a predetermined value. In some embodiments, the control value is an average value of sperm motility measured from healthy sperm cells obtained from one or more individual donors. In some embodiments, the healthy sperm cells have not contacted the formulation. In some embodiments, the control value is determined in vitro.

In some embodiments of the formulations of the disclosure for use in promoting contraception, the formulation contacts a sperm cell for less than 2 minutes. In some embodiments, the formulation is administered to the subject less than 30 minutes prior to intercourse. In some embodiment, the formulation is administered to the subject between 15 and 20 minutes prior to intercourse.

In some embodiments of the formulations of the disclosure for use in promoting contraception, the formulation is administered locally. In some embodiments, the formulation is administered topically. In some embodiments, the subject is male. In some embodiments, the formulation is administered intravaginally. In some embodiments, the subject is female.

In some embodiments of the methods of the disclosure, the method further comprises administering an effective amount of niclosamide effective to treat or to prevent an infection. In some embodiments, the administration of the effective amount of niclosamide effective to treat or to prevent an infection is systemic. In some embodiments, the niclosamide is formulated for oral administration. In some embodiments, the niclosamide is formulated as a pill, capsule or tablet. In some embodiments, the niclosamide is formulated as a liquid. In some embodiments, the niclosamide is formulated for intravenous administration. In some embodiments, the administration of the effective amount of niclosamide effective to treat or to prevent an infection is local. In some embodiments, the niclosamide is formulated as a semisolid. In some embodiments, of the method of the disclosure, the niclosamide is formulated as a gel or a cream.

In some embodiments of the methods of contraception of the disclosure, the formulation as disclosed herein, comprises the niclosamide and wherein the formulation is administered in an amount effective to treat or to prevent an infection.

In some embodiments of the methods of contraception of the disclosure the method comprising administering to the subject a first amount of the formulation as disclosed herein, effective for contraception and a second amount of the formulation as disclosed herein, effective to treat or to prevent an infection. In some embodiments, the first amount of the formulation comprises a concentration of niclosamide of 100 mM. In some embodiments, the second amount of the formulation comprises a concentration of niclosamide sufficient to disrupt transcription of a nucleic acid sequence encoding a viral protein. In some embodiments, the second amount of the formulation comprises a concentration of niclosamide of between 0.1 μM and 30 μM, inclusive of the endpoints. In some embodiments, the first amount and the second amount are administered simultaneously. In some embodiments, the first amount and the second amount are administered sequentially. In some embodiments, the first amount and the second amount are administered by the same route. In some embodiments, the first amount and the second amount are not administered by the same route.

In some embodiments of the methods of the disclosure, the infection is transduced by a virus or a bacteria.

In some embodiments of the methods of the disclosure, the infection is transduced by a virus. In some embodiments, the virus is one or more of Herpes-Simplex virus (HSV), Human immunodeficiency virus (HIV), Coronavirus, Severe acute respiratory syndrome coronavirus (SARS-CoV-1), Severe acute respiratory syndrome coronavirus (SARS-CoV-2), Human coronavirus-Subtype OC43 (HCoV-OC43), Middle eastern respiratory syndrome corona virus (MERS-CoV), Vesicular stomatitis virus—Zaire Ebola virus (VZVEBOV), Dengue virus (DENV), Powassan virus (POWV) (NCT03760666), enterovirus (EV), Influenza A virus (FluAV), Human metapneumovirus (HMPV), Rabies virus (RABV), Rift valley fever virus (RVFV), Zika virus (ZIKV), West-nile virus (WNV), Yellow fever virus (YFV), Sindbis virus (SINV), Junin virus (JUNV), lassa Mammarenavirus (LASV) and lymphocytic choriomeningitis virus (LCMV) or a combination thereof. In some embodiments, the virus is a drug-resistant virus. In some embodiments, the virus is a retrovirus. In some embodiments, the infection is sexually-transmitted. In some embodiments, the viral infection is transduced by Human immunodeficiency virus-1 (HIV-1) or Human immunodeficiency virus-2 (HIV-2). In some embodiments, the viral infection is transduced by a Herpes simplex virus (HSV) or herpes simplex virus-2 (HSV-2).

In some embodiments of the methods of the disclosure, the infection is transduced by a bacterium. In some embodiments of the method of the disclosure, the infection is transduced by Mycobacterium tuberculosis.

The disclosure provides a method of treating or preventing a viral infection, comprising administering to a subject an effective amount of the formulation as disclosed herein, wherein the formulation inhibits viral replication, thereby treating or preventing a viral infection.

In some embodiments of the methods of the disclosure, the formulation comprises niclosamide at a concentration of: a) between 0.01 μM and 20 μM; b) between 0.04 μM and 1.1 μM; c) between 0.01 μM and 0.44 μM; d) between 0.04 μM and 0.30 μM; e) between 0.12 μM and 0.10 μM; 0 between 0.3 μM and 5 μM; or g) between 2.5 μM and 5 μM.

In some embodiments of the methods of the disclosure, the formulation comprises niclosamide at a concentration of 0.4 μM. In some embodiments of the methods of the disclosure, the formulation comprises niclosamide at a concentration of 1.1 μM.

In some embodiments of the methods of the disclosure, the formulation comprises niclosamide at a concentration of between 0.01 μM and 20 μM. In some embodiments, the formulation comprises niclosamide at a concentration of between 0.04 μM and 1.1 μM. In some embodiments, the formulation comprises niclosamide at a concentration of between 0.01 μM and 0.44 μM. In some embodiments, the formulation comprises niclosamide at a concentration of between 0.04 μM and 0.30 μM. In some embodiments, the formulation comprises niclosamide at a concentration of between 0.12 μM and 0.10 μM. In some embodiments, the formulation comprises niclosamide at a concentration of 0.3 μM and 5 μM. In some embodiments, the formulation comprises niclosamide at a concentration of 2.5 μM and 5 μM.

In some embodiments of the methods of the disclosure, the formulation comprises niclosamide at a concentration of 0.4 μM. In some embodiments, the formulation comprises niclosamide at a concentration of 1.1 μM.

In some embodiments of the methods of the disclosure, the method further comprises administering a therapeutically amount of a second therapeutic agent, wherein the second therapeutic agent comprises an anti-viral agent, an anti-bacterial agent, an anti-fungal agent or an anti-parasitic agent. In some embodiments, the second therapeutic agent is Obatoclax, Emetine, Homoharringtonin, Brequinar, Suramin or a combination thereof.

In some embodiments of the methods of the disclosure, the subject is human. In some embodiments, the subject is female. In some embodiments, the subject is male.

In some embodiments of the methods of the disclosure, the formulation is administered locally. In some embodiments, the formulation is administered topically, intrarectally, intramuscularly or intravaginally.

The disclosure provides a formulation of the disclosure for use in treating or preventing an infection transduced by a virus or a bacteria, comprising administering an effective amount of the formulation to the subject, wherein, upon contacting an infectious agent, the formulation prevents transmission, infection, replication, survival or growth of the virus or bacteria in the subject.

In some embodiments of the methods of the disclosure, the infection is transduced by a virus. In some embodiments, the virus is one or more of Herpes-Simplex virus (HSV), Human immunodeficiency virus (HIV), Coronavirus, Severe acute respiratory syndrome coronavirus (SARS-CoV-1), Severe acute respiratory syndrome coronavirus (SARS-CoV-2), Human coronavirus-Subtype OC43 (HCoV-OC43), Middle eastern respiratory syndrome corona virus (MERS-CoV), Vesicular stomatitis virus—Zaire Ebola virus (VZVEBOV), Dengue virus (DENV), Powassan virus (POWV) (NCT03760666), enterovirus (EV), Influenza A virus (FluAV), Human metapneumovirus (HMPV), Rabies virus (RABV), Rift valley fever virus (RVFV), Zika virus (ZIKV), West-nile virus (WNV), Yellow fever virus (YFV), Sindbis virus (SINV), Junin virus (JUNV), lassa Mammarenavirus (LASV) and lymphocytic choriomeningitis virus (LCMV) or a combination thereof. In some embodiments, the virus is a drug-resistant virus. In some embodiments, the virus is a retrovirus. In some embodiments, the infection is sexually-transmitted. In some embodiments, the viral infection is transduced by Human immunodeficiency virus-1 (HIV-1) or Human immunodeficiency virus-2 (HIV-2). In some embodiments, the viral infection is transduced by a Herpes simplex virus (HSV) or herpes simplex virus-2 (HSV-2).

In some embodiments of the formulations for use in treating or preventing an infection, the infection is transduced by a bacterium. In some embodiments of the method of the disclosure, the infection is transduced by Mycobacterium tuberculosis.

In some embodiments of the formulations for use in treating or preventing an infection, the formulation comprises niclosamide at a concentration of between 0.01 μM and 20 μM. In some embodiments, the formulation comprises niclosamide at a concentration of between 0.04 μM and 1.1 μM. In some embodiments, the formulation comprises niclosamide at a concentration of between 0.01 μM and 0.44 μM. In some embodiments, the formulation comprises niclosamide at a concentration of between 0.04 μM and 0.30 μM. In some embodiments, the formulation comprises niclosamide at a concentration of between 0.12 μM and 0.10 μM. In some embodiments, the formulation comprises niclosamide at a concentration of 0.3 μM and 5 μM. In some embodiments, the formulation comprises niclosamide at a concentration of 2.5 μM and 5 μM.

In some embodiments of the formulations for use in treating or preventing an infection, the formulation comprises niclosamide at a concentration of 0.4 μM. In some embodiments, the formulation comprises niclosamide at a concentration of 1.1 μM.

In some embodiments of the formulations for use in treating or preventing an infection, the use further comprises administering a therapeutically amount of a second therapeutic agent, wherein the second therapeutic agent comprises an anti-viral agent, an anti-bacterial agent, an anti-fungal agent or an anti-parasitic agent. In some embodiments, the second therapeutic agent is Obatoclax, Emetine, Homoharringtonin, Brequinar, Suramin or a combination thereof.

In some embodiments of the formulations for use in treating or preventing an infection, the subject is human. In some embodiments, the subject is female. In some embodiments, the subject is male.

In some embodiments of the formulations for use in treating or preventing an infection, the formulation is administered locally. In some embodiments, the formulation is administered topically, intrarectally, intramuscularly or intravaginally.

In some embodiments of the formulations for use in treating or preventing an infection, the formulation is for use in treating or preventing an infection transduced by a virus that communicable by means other than sexual contact.

In some embodiments of the formulations for use in treating or preventing an infection, contacting an infectious agent comprises the subject contacting directly or indirectly an organism infected with the virus or contaminated by the virus. In some embodiments, the organism presents one or more sign(s) or symptom(s) of an infection. In some embodiments, the organism does not present a sign or a symptom of an infection. In some embodiments, the organism is an asymptomatic carrier of the virus.

In some embodiments of the formulations for use in treating or preventing an infection, contacting comprises communication of the virus through air or through fluid media. In some embodiments, the fluid comprises a bodily fluid or particulate thereof from the infected or contaminated organism. In some embodiments, the bodily fluid particulate comprises an exhaled or an expelled droplet. In some embodiments, the bodily fluid particulate comprises an aerosolized droplet. In some embodiments, the bodily fluid comprises sputum, saliva, blood, plasma, serum, lymph fluid, tears, sweat, urine or feces.

In some embodiments of the formulations for use in treating or preventing an infection, the virus is communicated to the subject from across a physical distance of between 0.1 and 12 feet from the organism. In some embodiments, the virus is communicated to the subject from across a physical distance of 6 feet or less from the organism.

In some embodiments of the formulations for use in treating or preventing an infection, the virus survives, remains viable or retains an infectious activity on a surface for between 0.1 minute and 6 months. In some embodiments, the surface comprises a biological surface. In some embodiments, the surface comprises one or more of a plant, a tree, a crop or a component thereof, skin, hair and nails. In some embodiments, the surface comprises one or more of silk, cotton, cellulose, cork, wool, wood, cardboard, latex, rubber and paper.

In some embodiments of the formulations for use in treating or preventing an infection, the surface does not comprise a biological surface. In some embodiments, the surface comprises an organic surface. In some embodiments, the surface comprises one or more of carbon fiber, a plastic and synthetic fiber. In some embodiments, the surface comprises an inorganic surface. In some embodiments, the surface comprises one or more of a metal, silicone and glass.

In some embodiments of the formulations for use in treating or preventing an infection, the organism infected with the virus or contaminated with the virus is a human.

In some embodiments of the formulations for use in treating or preventing an infection, the organism infected with the virus or contaminated with the virus is not a human. In some embodiments, the organism is a mammalian, an avian, a reptilian, an amphibian, a crustacean, an arthropod or a chordata organism. In some embodiments, the virus is a zoonotic virus. In some embodiments, contacting comprises consumption or handling of the organism by the subject. In some embodiments, the organism is a domesticated animal. In some embodiments of the formulations of the disclosure for use in treating or preventing an infection transduced by a virus, the domesticated animal is a pet or an ornamental animal. In some embodiments of the formulations of the disclosure for use in treating or preventing an infection transduced by a virus, the pet or ornamental animal is a dog, a cat, a bird, a reptile, a rodent including but not limited to a mouse, a rat, a rabbit, a hare and a hamster. In some embodiments, the domesticated organism is a cow, a goat, a chicken, a turkey, or a pig. In some embodiments, the organism is a live-stock animal. In some embodiments, the live-stock organism is a cow, a goat, a chicken, a turkey, or a pig. In some embodiments, the organism is a wild animal. In some embodiments, the wild animal organism is a non-human primate, a cetacean, a mammal, a bat, or a bird. In some embodiments, the wild animal organism or a part thereof is used for medicinal, ceremonial or ornamental purposes. In some embodiments, the wild animal organism or a part thereof contacts a component of the human food chain or, while contaminated and/or infectious, contacts an area within 5 miles of a human domicile.

In some embodiments of the formulations for use in treating or preventing an infection, the contacting occurs within a distance of 5 miles or less of: i) a human dwelling, ii) a laboratory or a research facility, iii) a market, store, or retail location, iv) a zoo, game reserve, wildlife reserve, land managed for wildlife protection or wildlife sanctuary, v) a farm, a field, or an agricultural location, vi) a hotel, a lodge, a resort or a site for an ecotourism activity, vii) a source of water, a well or a barrel maintained for drinking water, a stream, a river, a lake and an ocean, and/or viii) an airplane, a ship, a boat, a bus, a train, a car, a truck, or any other means of public, commercial or personal transportation.

In some embodiments of the formulations for use in treating or preventing an infection, the organism is maintained in one or more of i) a human dwelling, ii) a laboratory or a research facility, iii) a market, store, or retail location, iv) a zoo, game reserve, wildlife reserve, land managed for wildlife protection or wildlife sanctuary, v) a farm, a field, or an agricultural location, vi) a hotel, a lodge, a resort or a site for an ecotourism activity, vii) a source of water, a well or a barrel maintained for drinking water, a stream, a river, a lake and an ocean, and/or viii) an airplane, a ship, a boat, a bus, a train, a car, a truck, or any other means of public, commercial or personal transportation.

In some embodiments of the formulations for use in treating or preventing an infection, contacting the organism is intentional. In some embodiments, the organism is personal property, state property, communal property, a hunting target, a food source, a research subject, a pet, a native species, an invasive species, a prey of any one of the foregoing or a predator of any one of the foregoing. In some embodiments, contacting the organism is unintentional. In some embodiments, the organism is a native species, an invasive species or a predator of the subject.

In some embodiments of the formulations for use in treating or preventing an infection, the virus has a mutation rate of between 10⁻⁸ and 10⁻⁴ mutations per nucleotide of the genome per replication cycle. In some embodiments of the formulations for use in treating or preventing an infection, the virus has a mutation rate of between 10⁻⁵ and 10⁻² nucleotide substitutions per site per year. In some embodiments of the formulations for use in treating or preventing an infection, wherein the virus has a mutation rate of between 0.80×10⁻³ and 2.38×10⁻³ nucleotide substitutions per site per year.

In some embodiments of the formulations for use in treating or preventing an infection, the virus has a mutation rate of between 1.16×10⁻³ and 3.30×10⁻³ non-synonymous nucleotide substitutions per site per year. In some embodiments of the formulations for use in treating or preventing an infection, the virus has a mutation rate of between 1.67×10⁻³ and 4.67×10⁻³ synonymous nucleotide substitutions per site per year.

In some embodiments of the formulations for use in treating or preventing an infection, the virus presents antigenic drift. In some embodiments, a genomic sequence of the virus mutates or undergoes reassortment. In some embodiments, the genomic sequence of the virus mutates at a rate higher than 10⁻⁶ mutations per nucleotide of the genome per replication cycle. In some embodiments of the formulations for use in treating or preventing an infection, wherein the genomic sequence of the virus undergoes reassortment more frequently than 40%.

In some embodiments of the formulations for use in treating or preventing an infection, wherein the infection has an incubation period of between 1 and 15 days. In some embodiments, the infection has an incubation period of between 0.5 and 15 days. In some embodiments, the infection has an incubation period of between 0.5 and 1.5 days. In some embodiments, the infection has an incubation period of between 5 and 15 days.

In some embodiments of the formulations for use in treating or preventing an infection, the virus has a reproductive number (R0) of between 0.9 and 18. In some embodiments, the virus has a R0 of between 1.5 and 3.5. In some embodiments, the virus has a R0 of between 2 and 5.

In some embodiments of the formulations for use in treating or preventing an infection, the virus is an RNA virus. In some embodiments, the RNA virus is a positive-strand RNA virus. In some embodiments, the positive-strand RNA virus belongs to the family of Picornaviridae, Astroviridae, Caliciviridae, Hepeviridae Flaviviridae, Togaviridae, Arteriviridae, or Coronaviridae. In some embodiments, the positive-strand RNA virus belongs to the family of Coronaviridae.

In some embodiments of the formulations for use in treating or preventing an infection, the Coronaviridae virus is Bat coronavirus CDPHE15, Bat coronavirus HKU10, Rhinolophus ferrumequinum alphacoronavirus HuB-2013, Human coronavirus 229E, Lucheng Rn rat coronavirus, Ferret coronavirus, Mink coronavirus 1, Miniopterus bat coronavirus 1, Miniopterus bat coronavirus HKU8, Myotis ricketti alphacoronavirus Sax-2011, Nyctalus velutinus alphacoronavirus SC-2013, Porcine epidemic diarrhea virus, Scotophilus bat coronavirus 512, Rhinolophus bat coronavirus HKU2, Human coronavirus NL63, NL63-related bat coronavirus strain BtKYNL63-9b, Human coronavirus OC43, China Rattus coronavirus HKU24, Human coronavirus HKU1, Murine coronavirus—type species, Bat Hp-betacoronavirus Zhejiang2013, Hedgehog coronavirus 1, Middle East respiratory syndrome-related coronavirus (MERS-CoV), Pipistrellus bat coronavirus HKU5, Tylonycteris bat coronavirus HKU4, Rousettus bat coronavirus GCCDC1, Rousettus bat coronavirus HKU9, Severe acute respiratory syndrome-related coronavirus, Wigeon coronavirus HKU20, Bulbul coronavirus HKU11—type species, Porcine coronavirus HKU15, Munia coronavirus HKU13, White-eye coronavirus HKU16, Night heron coronavirus HKU19, Common moorhen coronavirus HKU21, Beluga whale coronavirus SW1 or Avian coronavirus—type species.

In some embodiments of the formulations for use in treating or preventing an infection, the Coronaviridae virus is Middle East respiratory syndrome-related coronavirus (MERS-CoV), Severe acute respiratory syndrome coronavirus (SARS-CoV) or Severe acute respiratory syndrome coronavirus 2 (SARS-CoV-2). In some embodiments, the Coronaviridae virus is Severe acute respiratory syndrome coronavirus 2 (SARS-CoV-2).

In some embodiments of the formulations for use in treating or preventing an infection, wherein the RNA virus is a negative-strand RNA virus. In some embodiments, the negative-strand RNA virus belongs to the family of Orthomyxoviridae, Paramyxoviridae, Rhabdoviridiae, Filoviruses, or Arenaviruses.

In some embodiments of the formulations for use in treating or preventing an infection, the negative-strand virus is Influenza virus, Sendai virus, Human parainfluenza virus 1 (hPIV1), Simian virus 5 (SV5, PIV5), Mumps virus, Newcastle disease virus (NDV), Measles virus, Rinderpest virus, Respiratory syncytial virus (RSV), Vesicular stomatitis virus (VSV), Rabies virus, Ebola virus, Marburg virus, Lymphocytic choriomeningitis virus (LCMV), Junin virus, or Lassa fever virus.

In some embodiments of the formulations for use in treating or preventing an infection, wherein the virus is a retrovirus. In some embodiments, the retrovirus is a Lentivirus, Alpharetrovirus, Betaretrovirus, Deltaretrovirus, Gammaretrovirus or Epsilonretrovirus. In some embodiments, the retrovirus is a Simian immunodeficiency virus (SIV), Human immunodeficiency virus-1 (HIV-1), HIV-2, Feline immunodeficiency virus (FIV), Equine infectious anemia virus (EIAV), Mouse mammary tumor-like virus (MMTV), Mason-Pfizer monkey virus (MPMV), Respiratory syncytial virus RSV, bovine leukemia virus (BLV), Human T-cell leukemia virus-1 (HTLV-1), HTLV-2, Murine leukemia virus (MuLV), Gibbon ape leukemia virus (GALV).

In some embodiments of the formulations for use in treating or preventing an infection, the virus is a DNA virus. In some embodiments, the DNA virus is Adenovirus, infectious canine hepatitis virus, Papillomavirus, polyomaviridae, simian vacuolating virus, Parvovirus B19, canine parvovirus, Herpes simplex virus, varicella-zoster virus, cytomegalovirus, Epstein-Barr virus, Smallpox virus, cow pox virus, sheep pox virus, monkey pox virus, vaccinia virus, Torque teno virus.

In some embodiments of the formulations for use in treating or preventing an infection, the subject is human. In some embodiments, the subject is female. In some embodiments, the subject is male. In some embodiments, the subject is a neonate. In some embodiments, the subject is an infant. In some embodiments, the subject is a child. In some embodiments, the subject is an adult. In some embodiments, the subject is a senior adult. In some embodiments, the subject is an elderly adult.

In some embodiments of the formulations for use in treating or preventing an infection, the subject is not admitted to a hospital or comparable facility to receive professional medical care. In some embodiments, the subject is admitted to a hospital or comparable facility to receive professional medical care.

In some embodiments of the formulations for use in treating or preventing an infection, the subject is not admitted to a residential care facility to receive routine medical care. In some embodiments, the subject is admitted to a residential care facility to receive routine medical care.

In some embodiments of the formulations for use in treating or preventing an infection, the subject is not connected to a ventilator. In some embodiments, the subject is connected to a ventilator. In some embodiments, the subject is in a coma. In some embodiments, the subject is not connected to a ventilator.

In some embodiments of the formulations for use in treating or preventing an infection, the subject is sentenced to a term or resident in a prison, jail, correctional facility or comparable facility.

In some embodiments of the formulations for use in treating or preventing an infection, the subject is deemed an essential worker by a governmental authority or an employer.

In some embodiments of the formulations for use in treating or preventing an infection, the subject has an increased risk of exposure to the virus or an increased risk of infection from the virus when compared to an average citizen. In some embodiments, the subject is one or more of an essential worker, an employee of a healthcare facility, an employee of an agricultural producer, an employee of a meat or diary processing plant, an employee of a food manufacturer, an employee of a food distributor, an employee of a food seller, an employee of a governmental agency, an employee of an emergency or essential agency, an emergency first-responder, an employee of a biological research, development or manufacturing company, an employee of a pharmacy, an employee of a warehouse owner, as student, a teacher and an employee of a funeral home.

As used herein, the term “about,” unless indicated otherwise, refers to the recited value, e.g., amount, dose, temperature, time, percentage, etc., ±10%, ±9%, ±8%, ±7%, ±6%, ±5%, ±4%, ±3%, ±2%, or ±1%.

### BRIEF DESCRIPTION OF THE DRAWINGS

FIG. 1 is a graph depicting the dose dependent effect of niclosamide dissolved in DMSO, on sperm motility. Fresh human semen samples were mixed with the different concentrations of niclosamide in vitro, followed by analysis of sperm motility within 2 minutes. The x-axis depicts the different concentrations of niclosamide mixed with the semen sample. The x-axis, from Left to Right: the gray bar depicts an untreated semen sample, the blue bar depicts a vehicle control, and the eight pink bars depict the increasing concentrations of niclosamide tested. There is 100% inhibition of sperm motility relative to the control treated sample at the highest doses of 10 mM and 50 mM niclosamide. The y-axis depicts the percentage (%) inhibition in sperm motility of each semen sample treated with a concentration of niclosamide o normalized to the sperm motility of a control sample. Change in sperm motility relative to control treated sample is shown as mean +/−standard error of the mean (S.E.M.) for the aggregate of individual experiments.

FIGS. 2A-2B are graphs comparing the ability of inhibiting sperm motility and pH buffering capacity of a niclosamide formulation of the disclosure with a commercially available contraceptive formulation, Phexxi. Fresh isolated human semen was mixed with either a niclosamide gel formulation (as depicted in Table 16, comprising 5% w/w niclosamide, 65% w/w PEG-400, 0.5% w/w carbopol 980 and 0.2% w/w benzyl alchohol), or Phexxi, as indicated in the graphs. FIG. 2A depicts the change in number of motile sperms (normalized to control) on the y-axis, and the corresponding human semen to formulation ratios on the x-axis, from left to right. FIG. 2B depicts the change in the pH of the semen and formulation mixture on the y-axis, and the corresponding human semen to formulation ratios on the x-axis, from left to right. Change in number of motile sperms and change in pH of the semen and formulation mixture are shown as mean +/−standard error of the mean (S.E.M.) for the aggregate of individual experiments.

FIG. 3 is a graph depicting luciferase kinetics of Vero E6 cells infected with nano luciferase reporter SARS-CoV-2. The Vero E6 cells were infected at a multiplicity of infection (MOI) of 0.3. Luciferase activities were measured at indicated timepoints post-infection. The dotted line indicates background level of luciferase signal from cells without viral infection. Results from triplicate experiments were presented with bars representing standard deviations.

FIG. 4 is a graph depicting antiviral activity of niclosamide in cell culture. Vero E6 cells were infected with nano luciferase reporter SARS-CoV-2 (MOI of 0.1) in the presence of niclosamide. At 24 h post-infction, luciferase activities were measured to estimate the EC50 value. The hill slope of the curve is also indicated. Results from triplicate experiments were presented with bars representing standard deviations

### DETAILED DESCRIPTION

Provided herein, is a method of treating or preventing an infection by a virus in a subject having or at risk for a viral infection and/or inhibiting or preventing viral replication of a virus in a subject having or at risk for a viral infection, comprising administering to a subject an effective amount of a formulation comprising an amount of niclosamide and one or more of a humectant, a lubricant, a solvent, an osmolality modulator, a pH modulator, a viscosity modulator and a preservative, wherein the formulation inhibits viral replication of the virus, optionally thereby treating or preventing a viral infection.

In some embodiments of the method of the disclosure, the virus is a Herpes-Simplex virus (HSV), Human immunodeficiency virus (HIV), Coronavirus, Severe acute respiratory syndrome coronavirus (SARS-CoV-1), Severe acute respiratory syndrome coronavirus (SARS-CoV-2), Human coronavirus-Subtype OC43 (HCoV-OC43), Middle eastern respiratory syndrome corona virus (MERS-CoV), Vesicular stomatitis virus—Zaire Ebola virus (VZVEBOV), Dengue virus (DENV), Powassan virus (POWV), Enterovirus (EV), Influenza A virus (FluAV), Human metapneumovirus (HMPV), Rabies virus (RABV), Rift valley fever virus (RVFV), Zika virus (ZIKV), West-nile virus (WNV), Yellow fever virus (YFV), Sindbis virus (SINV), Junin virus (JUNV), Lassa mammarenavirus (LASV), or Lymphocytic choriomeningitis virus (LCMV). In some embodiments of the method of the disclosure, the virus is a Coronavirus. In some embodiments of the method of the disclosure, the virus is a SARS-CoV-2.

In some embodiments of the method of the disclosure, the administering step results in a systemic concentration of niclosamide at about 150 mM in the subject. In some embodiments of the method of the disclosure, the administering step results in a systemic concentration of niclosamide of less than 150 mM in the subject. In some embodiments of the method of the disclosure, the administering step results in a systemic concentration of niclosamide of less than 150 mM, less than 100 mM, less than 50 mM, less than 25 mM, less than 10 mM or less than 5 mM in the subject. In some embodiments of the method of the disclosure, the systemic concentration of niclosamide is concentration of niclosamide in blood, serum or plasma of the subject.

In some embodiments of the method of the disclosure, the administration step results in a concentration of niclosamide at a site of administration of the formulation in the subject, of about 100 nM to about 1 μM. In some embodiments of the method of the disclosure, the administration step results in a concentration of niclosamide at a site of administration of the formulation in the subject, of about 100 nM to about 200 nM, about 200 nM to about 300 nM, about 300 nM to about 400 nM, about 400 nM to about 500 nM, about 500 nM to about 600 nM, about 600 nM to about 700 nM, about 700 nM to about 800 nM, about 800 nM to about 900 nM or about 900 nM to about 1 μM. In some embodiments of the method of the disclosure, the administration step results in a concentration of niclosamide at a site of administration of the formulation in the subject, of about 100 nM to about 500 nM. In some embodiments of the method of the disclosure, the administration step results in a concentration of niclosamide at a site of administration of the formulation in the subject, of about 100 nM to about 250 nM. In some embodiments of the method of the disclosure, the administration step results in a concentration of niclosamide at a site of administration of the formulation in the subject, of about 100 nM to about 150 nM. In some embodiments of the method of the disclosure, the administration step results in a concentration of niclosamide at a site of administration of the formulation in the subject, at about 150 nM.

In some embodiments of the method of the disclosure, the formulation is administered locally. In some embodiments of the method of the disclosure, the formulation is administered topically, intrarectally, intramuscularly or intravaginally. In some embodiments of the method of the disclosure, the subject is human.

Provided herein are methods and formulations for use in the methods, for treating or preventing infection

An ideal contraceptive would be one that is: (1) easily acquired and administered, (2) inexpensive, (3) lacks systemic effects, (4) lacks the ability to cause irritation to the subject or his/her/their partner, (5) maintains normal flora and pH of the skin, vagina and/or rectum and, (6) provides contraceptive effects. Current contraceptive methods do not meet all of these requirements. Hormonal contraceptives, while convenient to use, have systemic effects and require regular visits to clinicians for the monitoring of possible severe side-effects (e.g. edema, weigh gain, abdominal bloating, nausea, depression, acne, hirsutism, vaginal bleeding, and adverse effects on plasma lipoprotein profiles). Barrier contraceptives for females such as the diagram and cervical caps require fitting by a clinician. Currently available and widely used spermicides have been shown to cause irritation in a large segment of the user population, due to the destruction of the normal vaginal flora, and increase susceptibility to sexually transmitted diseases and conditions caused by an increase of the vaginal pH.

Additional characteristics that have not yet before been sought after are contraceptive methods for male use. The formulations of the disclosure enable men to participate in contraception in a similarly safe, effective, and non-hormonal method. For use as a contraceptive the formulations of the disclosure may be applied topically to male subjects alone or in combination with a barrier method (e.g. a condom).

Non-hormonal formulations that can reduce or inhibit sperm functionality (motility and live sperm count) thereby effectively reducing or preventing sperm entry into the uterine tract and fusion with an ova, without negatively effecting the health and comfort of the subject, are highly desired.

The disclosure is directed to formulations, characterized by effective concentration ranges of niclosamide, and a pharmaceutically acceptable carrier, that effectively reduces or inhibits sperm motility, after contacting a sperm cell. In some embodiments of the method of the disclosure, formulations of the disclosure may be in the form of a gel or cream formulation that is easily available and applicable by a subject.

Provided are formulations, comprising niclosamide, that can be in a gel or cream formulation, that completely inhibit sperm motility. Provided is a formulation comprising an amount of niclosamide and one or more of a humectant, a lubricant, a solvent, an osmolality modulator, a pH modulator, a viscosity modulator and a preservative. Provided is a method of contraception, comprising administering an effective amount of the formulation of the disclosure to the subject, wherein, upon contacting a sperm cell, the formulation inhibits or decreases one or more of sperm motility, sperm viability, and sperm metabolism, thereby decreasing the probability of conception. Provided is a formulation for use in a method of promoting contraception, wherein, the method comprises administering an effective amount of the formulation to the subject, wherein, upon contacting a sperm cell, the formulation inhibits or decreases one or more of sperm motility, sperm viability, and sperm metabolism, thereby decreasing the probability of conception. Provided is a use of the formulation of the disclosure, in the manufacture of a medicament for use in a method of promoting contraception in a subject.

In some embodiments of the formulations of the disclosure, the formulation has a viscosity of between about 180 cPs and about 58,000 cPs. In some embodiments of the formulations of the disclosure, the formulation has a viscosity of between about 180 cPs and about 16,000 cPs. In some embodiments of the formulations of the disclosure, the formulation has a viscosity of between about 700 cPs and about 58,000 cPs. In some embodiments of the formulations of the disclosure, the viscosity modulator is HEC, or carbopol, or polycarbophil, or pemulen. In some embodiments of the formulations of the disclosure, the formulation has a viscosity of between about 10,000 cPs and about 18,000 cPs. In some embodiments of the formulations of the disclosure, the viscosity modulator is carbopol or polycarbophil. In some embodiments of the formulations of the disclosure, the formulation has a viscosity of 7,00 cPs. In some embodiments of the formulations of the disclosure, the viscosity modulator is HEC. In some embodiments of the formulations of the disclosure, the formulation has a viscosity of 11,000 cPs. In some embodiments of the formulations of the disclosure, the viscosity modulator is carbopol. In some embodiments of the formulations of the disclosure, the formulation has a viscosity of 18,000 cPs. In some embodiments of the formulations of the disclosure, the viscosity modulator is polycarbophil. In some embodiments of the formulations of the disclosure, the formulation has a viscosity of 58,000 cPs. In some embodiments of the formulations of the disclosure, the viscosity modulator is pemulen.

### Niclosamide

Formulations of the disclosure comprise an amount of niclosamide.

niclosamide has the chemical formula: C₁₃H₈Cl₂N₂O₄, molecular weight of 327.12 g/mol and the chemical structure:

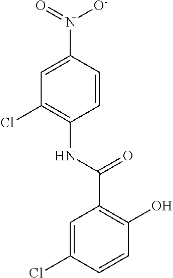

The use of niclosamide had been approved by the United States Federal Drug Administration (FDA) under the trade name “NICLOCIDE” as an orally administrated chewable tablet having 500 mg of the niclosamide active ingredient for the treatment of tapeworm infections. NICLOCIDE has been discontinued.

Formulation of niclosamide as a semi-solid or liquid presents unique challenges as niclosamide is difficult to maintain in solution or in any form other than a dry solid. The formulations of the disclosure not only overcome this challenge, but also provide a new route of administration and new uses of the formulations as a contraceptive for use by either a man or a woman. Thus, the formulations of the disclosure provide a unisex contraceptive formulation. Route of administration may vary depending on the sex of the subject using the formulation. However, the formulations of the disclosure are safe and effective for local administration, either topically or internally, when applied to a surface of a body cavity.

### Niclosamide Formulations

Formulations of the disclosure, including a semi-solid form, a gel, a lotion or a cream, may comprise a concentration of the niclosamide of between 1 mM and 10 mM, 10 mM and 100 mM, 10 mM and 20 mM, 20 mM and 30 mM, 30 mM and 40 mM, 40 mM and 50 mM, 50 mM and 60 mM, 60 mM and 70 mM, 70 mM and 80 mM, 80 mM and 90 mM, 90 mM and 100 mM, inclusive of the endpoints. In some embodiments, formulations of the disclosure may comprise a concentration of niclosamide of 4 mM. In some embodiments, formulations of the disclosure may comprise a concentration of niclosamide of 100 mM. In some embodiments, formulations of the disclosure may comprise a concentration of niclosamide of between 4 mM and 100 mM, inclusive of the endpoints.

In some embodiments of the formulations of the disclosure, including a semi-solid form, a gel, a lotion or a cream, the formulation may comprise an amount of niclosamide, of between 0.1% and 10%, of the total weight of the formulation, inclusive of the endpoints. In some embodiments of the formulations of the disclosure, including a semi-solid form, a gel, a lotion or a cream, the formulation may comprise an amount of niclosamide, of between 0.1% and 10%, 0.1% and 9%, 0.1% and 8%, 0.1% and 7%, 0.1% and 6%, 0.1% and 5%, 0.1% and 4%, 0.1% and 3%, 0.1% and 2%, and 0.1% and 1%, of the total weight of the formulation, inclusive of the endpoints. In some embodiments of the formulations of the disclosure, including a semi-solid form, a gel, a lotion or a cream, the formulations may comprise an amount of niclosamide, of between 0.1% and 1%, 0.1% and 0.9%, 0.1% and 0.8%, 0.1% and 8%, 0.1% and 0.7%, 0.1% and 0.6%, 0.1% and 0.5%, 0.1% and 0.4%, 0.1% and 0.3%, 0.1% and 0.2%, of the total weight of the formulation, inclusive of the endpoints. In some embodiments of the formulations of the disclosure, including a semi-solid form, a gel, a lotion or a cream, the formulation may comprise an amount of niclosamide, between 1% and 10%, 1% and 9%, 1% and 8%, 1% and 7%, 1% and 6%, 1% and 5%, 1% and 4%, 1% and 3%, 1% and 2%, of the total weight of the formulation, inclusive of the endpoints. In some embodiments of the formulations of the disclosure, the formulation may comprise an amount of the niclosamide, between 1% and 5% of the total weight of the formulation, including the end points. In some embodiments of the formulations of the disclosure, the formulation may comprise an amount of niclosamide, of 0.13% of the total weight of the formulation. In some embodiments of the formulations of the disclosure, the formulation may comprise an amount of niclosamide, of 3.27% of the total weight of the formulation. In some embodiments of the formulations of the disclosure, the formulation may comprise an amount of niclosamide, of 10% of the total weight of the formulation.

In some embodiments of the formulations of the disclosure, the formulation may comprise an amount of niclosamide, of between 0.1% and 10% of total weight of the formulation, inclusive of the endpoints. In some embodiments of the formulations of the disclosure, the formulation may comprise an amount of niclosamide, of between 0.1% and 5% of total weight of the formulation, inclusive of the endpoints. In some embodiments of the formulations of the disclosure, the formulation may comprise an amount of niclosamide, of between 1% and 5% of total weight of the formulation, inclusive of the endpoints. In some embodiments of the formulations of the disclosure, the formulation may comprise an amount of niclosamide, of between 3% and 4% of total weight of the formulation, inclusive of the endpoints. In some embodiments of the formulations of the disclosure, the formulation may comprise an amount of niclosamide, of about 3.27% of total weight of the formulation.

In some embodiments of the formulations of the disclosure, the amount of niclosamide, is between 3% and 7% of total weight of the formulation, inclusive of the endpoints. In some embodiments of the formulations of the disclosure, the amount of niclosamide, is between 3% and 6% of total weight of the formulation, inclusive of the endpoints. In some embodiments of the formulations of the disclosure, the amount of niclosamide, is about 5% of total weight of the formulation. In some embodiments of the formulations of the disclosure, the amount of niclosamide, is 5% of total weight of the formulation. In some embodiments of the formulations of the disclosure, the amount of niclosamide is about 3% of total weight of the formulation. In some embodiments of the formulations of the disclosure, the amount of niclosamide is 3% of total weight of the formulation. In some embodiments of the formulations of the disclosure, the formulation may comprise an amount of niclosamide, of about 3.27% of total weight of the formulation. In some embodiments of the formulations of the disclosure, the formulation may comprise an amount of niclosamide, of 3.27% of total weight of the formulation. In some embodiments of the formulations of the disclosure, the amount of niclosamide is 1% to 2%, 2% to 3%, 3% to 4% or 4% to 5% of total weight of the formulation, inclusive of the endpoints.

In some embodiments of the formulations of the disclosure, the formulation may comprise niclosamide, at a concentration that is between 4 mM and 500 mM, inclusive of the endpoints. In some embodiments of the formulations of the disclosure, the formulation may comprise niclosamide, at a concentration that is between 10 mM and 250 mM, inclusive of the endpoints. In some embodiments of the formulations of the disclosure, the niclosamide has a concentration of about 150 mM. In some embodiments of the formulations of the disclosure, the niclosamide has a concentration of between 5 mM to 25 mM, 25 mM to 50 mM, 50 mM to 75 mM, 75 mM to 100 mM, 100 mM to 125 mM, 125 mM to 150 mM, 150 mM to 175 mM, 175 mM to 200 mM, 200 mM to 225 mM, 225 mM to 250 mM, 250 mM to 275 mM, 275 mM to 300 mM, 300 mM to 325 mM, 325 mM to 350 mM, 350 mM to 375 mM, 375 mM to 400 mM, 400 mM to 425 mM, 425 mM to 450 mM, 450 mM to 475 mM or 475 mM to 500 mM, inclusive of the endpoints. In some embodiments of the formulations of the disclosure, the niclosamide has a concentration of about 150 mM. In some embodiments of the formulations of the disclosure, the niclosamide has a concentration of 150 mM. In some embodiments of the formulations of the disclosure, the formulation may comprise niclosamide, at a concentration of about 100 mM. In some embodiments of the formulations of the disclosure, the formulation may comprise niclosamide, at a concentration of 100 mM. In some embodiments of the formulations of the disclosure, the niclosamide formulation may be a gel or a cream administered by a local route (e.g., topically or intra-cavitally to a component of the male or female reproductive system) and at a concentration of 150 nM. In some embodiments of the formulations of the disclosure, the niclosamide formulation may be a gel or a cream administered by a local route (e.g., intramuscularly) to a subject in need thereof to achieve a local concentration of 150 nM.

In some embodiments of the formulations of the disclosure, including a semi-solid form, a gel, a lotion or a cream, the formulation may comprise an amount of niclosamide, at the concentration or percentage by weight values, as depicted in Tables 1 and 2.

In some embodiments of the formulations of the disclosure, including a semi-solid form, a gel, a lotion or a cream, the formulation one or more of a filler, an excipient, an anti-adherent, a humectant, a coloring agent, a glidant, a preservative, a sorbent, a bulking agent, a lubricating agent, an osmolality adjusting agent, an anti-oxidant, a vehicle, a binding agent, a disintegration agent, a buffering agent, a solvent, a viscosity agent, and a stability agent.

In some embodiments of the formulations of the disclosure, including a semi-solid form, a gel, a lotion or a cream, the formulation a humectant or a solvent including, but not limited to, saccharides and their derivatives (e.g., disaccharides: sucrose, lactose), polysaccharides and their derivatives (e.g., starches, cellulose or modified cellulose such as microcrystalline cellulose and cellulose ethers such as hydroxypropyl cellulose (HPC)), sugar alcohols (e.g., xylitol, sorbitol or maltitol), protein gelatin, and synthetic polymers (e.g., polyvinylpyrrolidone (PVP), polyethylene glycol (PEG)).

In some embodiments, the humectant, the lubricant or the solvent of the formulation of the disclosure comprise a synthetic polymer or water. In some embodiments, the humectant, the lubricant or the solvent comprise at least two monomers of polyethylene glycol (PEG). In some embodiments, the formulation may comprise a polyethylene glycol (PEG). In some embodiments of the formulations of the disclosure, the humectant, the lubricant or the solvent comprises at least two monomers of polyethylene glycol (PEG), optionally PEG-400, PEG-1000, or PEG-2000. In some embodiments, the humectant, the lubricant or the solvent comprise PEG-400. In some embodiments of the formulations of the disclosure, the formulation comprises an amount of PEG-400 between 0.1% and 75% of the total weight of the formulation, inclusive of the endpoints. In some embodiments of the formulations of the disclosure, the amount of PEG-400 is between 35% and 75% of the total weight of the formulation, inclusive of the endpoints. In some embodiments of the formulations of the disclosure, the amount of PEG-400 is between 35% and 65% of the total weight of the formulation, inclusive of the endpoints. In some embodiments of the formulations of the disclosure, the amount of PEG-400 is about 35% of the total weight of the formulation. In some embodiments of the formulations of the disclosure, the amount of PEG-400 is 35% of the total weight of the formulation. In some embodiments of the formulations of the disclosure, the amount of PEG-400 is about 65% of the total weight of the formulation. In some embodiments of the formulations of the disclosure, the amount of PEG-400 is 65% of the total weight of the formulation.

In some embodiments the formulation of the disclosure, the formulation comprises an amount of the humectant, the lubricant or the solvent of between 0.1% and 65% of the total weight of the formulation, inclusive of the endpoints. In some embodiments the formulation of the disclosure, the formulation comprises a solvent, wherein the solvent comprises water and wherein formulation comprises an amount of the water of between 10% and 90% of the total weight of the formulation, inclusive of the endpoints.

In some embodiments the formulation of the disclosure, the formulation comprises PEG-400 between 0.1% and 75%, 0.1% and 65%, 0.1% and 60%, 0.1% and 55%, 0.1% and 50%, 0.1% and 50%, 0.1% and 45%, 0.1% and 40%, 0.1% and 35%, 0.1% and 30%, 0.1% and 20%, 0.1% and 10% or 0.1% and 5%, inclusive of the endpoints. In some embodiments the formulation of the disclosure, the formulation comprises PEG-400 between 1% to 10%, 20% and 30%, 30% and 40%, 40% and 50%, 50% and 60%, 60% and 70%, 70% and 80% or 80% and 90%, inclusive of the endpoints. In some embodiments the formulation of the disclosure, the formulation comprises PEG-400 between 5% to 10%, 10% and 15%, 15% and 20%, 20% and 25%, 25% and 30%, 30% and 35%, 35% and 40%, 40% and 45%, 45% and 50%, 50% and 55%, 55% and 60%, 60% and 65%, 65% and 70% or 70% and 75%, inclusive of the endpoints.

In some embodiments the formulation of the disclosure, the formulation comprises PEG-400, of the amount or percentage by weight values, as depicted in Tables 1 and 2.

In some embodiments, the formulation comprises an amount of the humectant, the lubricant or the solvent of between 0.1% and 65% of the total weight of the formulation, inclusive of the endpoints. In some embodiments, the solvent comprises water and the formulation comprise an amount of the water of between 10% and 90% of the total weight of the formulation, inclusive of the endpoints. In some embodiments, the solvent comprises water and the formulation comprise an amount of the water of between 10% and 20%, 20% and 30%, 30% and 40%, 40% and 50%, 50% and 60%, 60% and 70%, 70% and 80% or 80% and 90% of the total weight of the formulation, inclusive of the endpoints. In some embodiments, the water is purified water.

In some embodiments the formulation of the disclosure, the formulation comprise an osmolality adjusting agent or osmolality modulator. In some embodiments, the formulations of the disclosure comprise an osmolality modulator selected from the group consisting of salt of aluminum, arginine, benzathine, calcium, chloroprocaine, choline, diethanolamine, ethanolamine, ethylenediamine, histidine, lithium, lysine, magnesium, meglumine, potassium, procaine, sodium, triethylamine, zinc or a combination thereof. In some embodiments, the osmolality modulator is a salt selected from the group consisting of acetate, glutamate, mucate, aspartate, glycolate, napsylate, benzenesulfonate, glycollylarsanilate, nitrate, benzoate, hexanoate, octanoate, chloroprocaine, besylate, hexylresorcinate, oleate, bicarbonate, hydrabamine, pamoate, bitartrate, hydroxynaphthoate, pantothenate, bromide, iodide, phosphate, camsylate, isethionate, polygalacturonate, carbonate, isethionate, propionate, chloride, lactate, salicylate, citrate, lactobionate, stearate, decanoate, malate, subacetate, edetate, maleate, succinate, estolate, mandelate, sulfate, esylate, mesylate, tartrate, fumarate, methylbromide, teoclate, gluceptate, methylnitrate, tosylate, gluconate, methylsulfate, triethiodide or a combination thereof.

In some embodiments of the formulations of the disclosure, the osmolality modulator is sodium chloride. In some embodiments, the formulation comprises an amount of the osmolality modulator of less than 5% of the total weight of the formulation. In some embodiments, the formulation comprises an amount of the osmolality modulator of less than 1% of the total weight of the formulation.

In some embodiments, the formulations of the disclosure, comprise sodium chloride, in an amount of 0.9%, 0.8%, 0.7%, 0.6%, 0.5%, 0.4%, 0.3%, 0.2% or 0.1%, of the total weight of the formulation.

In some embodiments, the formulations of the disclosure, comprise sodium chloride in an amount or percentage by weight, as depicted Tables 1 and 2.

In some embodiments, the formulations of the disclosure, comprise a pH modulator. In some embodiments, the formulations of the disclosure, comprise a pH modulator, wherein the pH modulator comprise one or more of lactic acid, citric acid monohydrate, potassium sodium tartrate, potassium bitartrate, and sodium hydroxide. In some embodiments, the formulations of the disclosure, comprise an amount of the pH modulator of less than 10% of the total weight of the formulation and wherein the pH modulator comprises lactic acid. In some embodiments, the formulations of the disclosure, comprise an amount of lactic acid of less than 5% of the total weight of the formulation and wherein the pH modulator comprises lactic acid or citric acid monohydrate. In some embodiments, the formulations of the disclosure, comprise an amount of the pH modulator of less than 1% of the total weight of the formulation and wherein the pH modulator comprises one or more of lactic acid, citric acid monohydrate, and sodium hydroxide. In some embodiments, the formulations of the disclosure, comprise an amount of the pH modulator of less than 0.5% of the total weight of the formulation and wherein the pH modulator comprises one or more of lactic acid, citric acid monohydrate, potassium bitartrate and sodium hydroxide.

In some embodiments of the formulations of the disclosure, the pH modulator comprises one or more of lactic acid, citric acid monohydrate, potassium sodium tartrate, potassium bitartrate and sodium hydroxide. In some embodiments, the formulation comprises an amount of the pH modulator of less than 10% of the total weight of the formulation and wherein the pH modulator comprises lactic acid, potassium sodium tartrate, citric acid monohydrate, potassium bitartrate and sodium hydroxide. In some embodiments, the formulation comprises an amount of pH modulator of less than 5% of the total weight of the formulation and wherein the pH modulator comprises citric acid monohydrate, potassium sodium tartrate, potassium bitartrate and sodium hydroxide. In some embodiments, the formulation comprises an amount of the pH modulator of less than 1% of the total weight of the formulation and wherein the pH modulator comprises one or more of potassium sodium tartrate, potassium bitartrate and sodium hydroxide. In some embodiments, the formulation comprises an amount of the pH modulator of less than 0.5% of the total weight of the formulation and wherein the pH modulator comprises one or more of potassium sodium tartrate, potassium bitartrate and sodium hydroxide.

In some embodiments, the formulations of the disclosure, comprise a pH modulating agent or buffer that is lactic acid. In some embodiments, the formulation of the disclosure comprise lactic acid in an amount of less than 10%, 9%, 8%, 7%, 6%, 5%, 4%, 3%, 2% or 1%, of the total weight of the formulation, including the end points. In some embodiments, the formulation of the disclosure, including a semi-solid form, a gel, a lotion or a cream, comprise lactic acid, in an amount of less than 10% of the total weight of the formulation.

In some embodiments, the formulations of the disclosure, comprise a pH modulating agent or buffer that is citric acid monohydrate. In some embodiments, the formulations of the disclosure, comprise citric acid monohydrate, in an amount of less than 5%, 4%, 3%, 2%, 1%, 0.9%, 0.8%, 0.7%, 0.6%, 0.5%, 0.4%, 0.3%, 0.2% or 0.1%, of the total weight of the formulation, including the end points. In some embodiments, the formulations of the disclosure, comprise citric acid monohydrate, in an amount of less than 5% of the total weight of the formulation.

In some embodiments, the formulations of the disclosure comprise a pH modulating agent or buffer that is potassium sodium tartrate. In some embodiments, the formulation of the disclosure comprise potassium sodium tartrate, in an amount of less than 0.5%, 0.4%, 0.3%, 0.2%, 0.1%, 0.09%, 0.08%, 0.07%, 0.06%, 0.05%, 0.04%, 0.03%, 0.02% or 0.01%, of the total weight of the formulation, including the end points. In some embodiments, the formulations of the disclosure, comprise potassium sodium tartrate, in an amount of less than 0.5% of the total weight of the formulation.

In some embodiments, the formulations of the disclosure comprise a pH modulating agent or buffer that is potassium bitartrate. In some embodiments, the formulation of the disclosure comprise potassium bitartrate, in an amount of less than 0.5%, 0.4%, 0.3%, 0.2%, 0.1%, 0.09%, 0.08%, 0.07%, 0.06%, 0.05%, 0.04%, 0.03%, 0.02% or 0.01%, of the total weight of the formulation, including the end points. In some embodiments, the formulations of the disclosure, comprise potassium bitartrate, in an amount of less than 0.5% of the total weight of the formulation.

In some embodiments, the formulations of the disclosure, comprise a pH modulating agent or buffer that is sodium hydroxide. In some embodiments, the formulations of the disclosure, comprise sodium hydroxide, in an amount of less than 1%, 0.9%, 0.8%, 0.7%, 0.6%, 0.5%, 0.4%, 0.3%, 0.2% or 0.1%, of the total weight of the formulation, including the end points. In some embodiments, the formulations of the disclosure, comprise sodium hydroxide, in an amount of less than 1% of the total weight of the formulation.

In some embodiments, the formulations of the disclosure, comprise a combination of pH modulating agents or buffers that an amount of lactic acid of 1% to 5% by weight of the formulation, an amount of citric acid monohydrate of 1% to 5% by weight of the formulation, an amount of potassium sodium tartrate of 0.1% to 0.5% by weight of the formulation, and an amount of sodium hydroxide of 0.1% to 0.5% by weight of the formulation. In some embodiments, the formulations of the disclosure, comprise a combination of pH modulating agents or buffers that an amount of lactic acid of 1% to 5% by weight of the formulation, an amount of citric acid monohydrate of 1% to 5% by weight of the formulation, an amount of potassium bitartrate of 0.1% to 0.5% by weight of the formulation, and an amount of sodium hydroxide of 0.1% to 0.5% by weight of the formulation.

In some embodiments, the formulations of the disclosure, comprise lactic acid, citric acid monohydrate, potassium bitartrate and sodium hydroxide, in an amount or percentage by weight, as depicted Tables 1 and 2.

In some embodiments, the formulations of the disclosure, comprise a viscosity modulator, wherein the viscosity modulator is a viscosity enhancer. In some embodiments, the viscosity enhancer comprises one or more of hydroxyethyl cellulose, alginic acid, polycarbophil and carbopol. In some embodiments, the formulations of the disclosure, comprise an amount of a viscosity enhancer of between 1% and 10% of the total weight of the formulation, inclusive of the endpoints. In some embodiments, the formulations of the disclosure, comprise an amount of a viscosity enhancer of between 1% and 5% of the total weight of the formulation, inclusive of the endpoints. In some embodiments, the formulations of the disclosure, comprise an amount of a viscosity enhancer of less than 5% of the total weight of the formulation. In some embodiments, the formulations of the disclosure, comprise an amount of a viscosity enhancer of 3% of the total weight of the formulation. In some embodiments, the formulation of the disclosure, the viscosity enhancer comprises hydroxyethyl cellulose.

In some embodiments, formulation of the disclosure, comprise a viscosity enhancer selected from the group consisting of agar, alamic acid, alginic acid, aluminum monostearate, attapulgite, activated, attapulgite colloidal activated, bentonite, bentonite, purified, bentonite magma, carbomer 910, carbomer 934, carbomer 934p, carbomer 940, carbomer 941, carbomer 1342, carbomer copolymer, carbomer homopolymer, carbomer interpolymer, carbopol, carboxymethylcellulose calcium, carboxymethylcellulose sodium, carboxymethylcellulose sodium 12, carrageenan, cellulose, dextrin, gelatin, gellan gum, guar gum, hydroxyethyl cellulose, hydroxypropyl cellulose, hypromellose (formerly hydroxypropyl methylcellulose), magnesium aluminum silicate, maltodextrin, methylcellulose, microcrystalline cellulose, pectin, polyethylene oxide, polyvinyl alcohol, povidone, propylene glycol alginate, polycarbophil, silicon dioxide, silicon dioxide, colloidal sodium alginate, starch corn, starch potato, starch tapioca, starch wheat, tragacanth and xanthan gum, or a combination thereof.

In some embodiments, the formulations of the disclosure, comprise hydroxyethyl cellulose in an amount of 1% to 10% of the total amount of the formulation. In some embodiments, the formulation comprises hydroxyethyl cellulose in an amount of 1%, 2%, 3%, 4%, 5%, 6%, 7%, 8%, 9% or 9% of the total amount of the formulation. In some embodiments, the formulations of the disclosure, comprise hydroxyethyl cellulose in an amount of 1% to 5% of the total amount of the formulation. In some embodiments, the formulations of the disclosure, comprise hydroxyethyl cellulose in an amount of 3% of the total weight of the formulation.

In some embodiments, the formulations of the disclosure, comprise alginic acid in an amount of less than 5%, less than 4%, less than 3%, less than 2% or less than 1% of the total weight of the formulation. In some embodiments, the formulations of the disclosure, comprise alginic acid in an amount of 4%, 3%, 2% or 1% of the total weight of the formulation. In some embodiments, the formulations of the disclosure, comprise alginic acid in an amount of less than 5% of the total weight of the formulation.

In some embodiments, the formulations of the disclosure, comprise polycarbophil in an amount of less than 5%, less than 4%, less than 3%, less than 2% or less than 1% of the total weight of the formulation. In some embodiments, the formulations of the disclosure, comprise polycarbophil in an amount of 4%, 3%, 2% or 1% of the total weight of the formulation. In some embodiments, the formulations of the disclosure, comprise polycarbophil in an amount of less than 5% of the total weight of the formulation.

In some embodiments, the formulations of the disclosure, comprise carbopol in an amount of less than 5%, less than 4%, less than 3%, less than 2% or less than 1% of the total weight of the formulation. In some embodiments, the formulations of the disclosure, comprise carbopol in an amount of 4%, 3%, 2% or 1% of the total weight of the formulation. In some embodiments, the formulations of the disclosure, comprise carbopol in an amount of less than 5% of the total weight of the formulation.

In some embodiments, the formulations of the disclosure, comprise hydroxyethyl cellulose in an amount of 1% to 5%; alginic acid in an amount of less than 5%; polycarbophil in an amount of less than 5%; and carbopol in an amount of less than 5%, of the total weight of the formulation.

In some embodiments, the formulation comprises an amount of a viscosity enhancer less than 1% of the total weight of the formulation. In some embodiments, the formulation comprises an amount of a viscosity enhancer of 0.1% to 1% of the total weight of the formulation. In some embodiments, the formulation comprises an amount of a viscosity enhancer of 0.1% to 0.2%, 0.2% to 0.3%, 0.3% to 0.4%, 0.4% to 0.5%, 0.5% to 0.6%, 0.6% to 0.7%, 0.7% to 0.8%, 0.8% to 0.9% or 0.9% to 1% of the total weight of the formulation. In some embodiments, the formulation comprises an amount of a viscosity enhancer of about 0.5% of the total weight of the formulation. In some embodiments, the formulation comprises an amount of a viscosity enhancer of 0.5% of the total weight of the formulation. In some embodiments, the viscosity enhancer comprises carbopol. In some embodiments, the carbopol is carbopol-980.

In some embodiments, the formulations of the disclosure, comprise viscosity enhancers hydroxyethyl cellulose, alginic acid, polycarbophil and carbopol in an amount or percentage by weight, as depicted Tables 1 and 2.

In some embodiments, the formulations of the disclosure, comprise a solvent or dispersion medium in an amount of between 10% to 90% of the total weight of the formulation, including the endpoints. In some embodiments, the formulation of the disclosure, comprise a solvent or dispersion medium that is water, ethanol, polyol (for example, glycerol, propylene glycol, and liquid polyethylene glycol, and the like), and suitable mixtures thereof. In some embodiments, the formulation of the disclosure, comprise a solvent or dispersion medium that is water. In some embodiments, the formulations of the disclosure, comprise water in an amount of between 10% and 90%.

In some embodiments, the formulations of the disclosure, comprise water in an amount of between 10% and 80%, 10% and 70%, 10% and 60%, 10% and 50%, 10% and 40%, 10% and 30%, and 10% and 20%, including the end points. In some embodiments, the formulations of the disclosure, comprise water in an amount of 10%, 20%, 30%, 40%, 50%, 60%, 70%, 80% and 90%, of the total weight of the formulation.

Niclosamide is naturally soluble in alcohol. However, the presence of high amounts of alcohols e.g., ethanol, in formulations known in the art for intravaginal or intrarectal administration, can cause several adverse effects including dryness, destruction of natural flora at site of administration and other issues. The niclosamide formulations of the present disclosure, comprise niclosamide dissolved in non-alcoholic solvents.

In some embodiments, the formulations of the disclosure are substantially free of alcohol (e.g., ethanol) or comprise alcohol(s) at a concentration of less that 1% of the total weight of the formulation. In some embodiments, the formulations of the disclosure comprise alcohol at a concentration of less than about 0.9%, less than about 0.8%, less than about 0.7%, less than about 0.6%, less than about 0.5%, less than about 0.4%, less than about 0.3%, less than about 0.2% or less than about 0.1% of the total weight of the formulation. In some embodiments, the formulations of the disclosure comprise alcohol at a concentration of less 0.9%, less 0.8%, less 0.7%, less 0.6%, less 0.5%, less 0.4%, less 0.3%, less 0.2% or less 0.1% of the total weight of the formulation. In some embodiments, the formulations of the disclosure comprise one or more alcohol at a concetartion of about 0.2% of the total weight of the formulation. In some embodiments, the formulations of the disclosure comprise one or more alcohol at a concentration of 0.2% of the total weight of the formulation. In some embodiments, the formulations of the disclosure comprise benzyl alcohol at a concentration of 0.2% of the total weight of the formulation. In some embodiments, the alcohol can be ethyl alcohol, methyl alcohol, propyl alcohol, butyl alcohol, benzyl alcohol or a combination thereof. In some embodiments, the formulations of the disclosure do not comprises of any amount or concentration or is essentially free of an alchohol selected from ethyl alcohol, methyl alcohol, propyl alcohol or butyl alcohol.

In some embodiments, formulations of the disclosure, including a semi-solid form, a gel, a lotion or a cream, comprise water in an amount or percentage by weight, as depicted in Tables 1 and 2.

In some embodiments, the formulations of the disclosure, comprise a preservative. In some embodiments, the preservative comprises one or more of benzyl alcohol, chlorhexidine gluconate and benzoic acid. In some embodiments of the formulations of the disclosure, the formulation comprises an amount of the preservative of less than 1% of the total weight of the formulation. In some embodiments, the formulation comprises an amount of the preservative of about 0.2% of the total weight of the formulation. In some embodiments, the formulation comprises an amount of the preservative of 0.2% of the total weight of the formulation. In some embodiments, the formulation comprises an amount of the preservative of less than 0.2% of the total weight of the formulation. In some embodiments, the formulations of the disclosure, comprise an amount of preservative between 0.1% o 0.2%, 0.2% to 0.3%, 0.3% to 0.4%, 0.4% to 0.5%, 0.5% to 0.6%, 0.6% to 0.7%, 0.7% to 0.8%, 0.8% to 0.9% or 0.95 to 0.99%, of the total weight of the formulation, including the end points. In some embodiments, the preservative comprises benzyl alcohol or benzoic acid. In some embodiments, the preservative comprises benzyl alcohol. In some embodiments, the preservative comprises benzoic acid.

In some embodiments, the formulations of the disclosure, comprise an amount of the preservative of less than 1% of the total weight of the formulation. In some embodiments, the preservative comprises benzyl alcohol. In some embodiments, the formulations of the disclosure, comprise an amount of the preservative of less than 0.2% of the total weight of the formulation. In some embodiments, the preservative comprises benzyl alcohol or benzoic acid. In some embodiments, the formulations of the disclosure, comprise an amount of the preservative of less than 0.12% of the total weight of the formulation. In some embodiments, the preservative comprises chlorhexidine gluconate.

In some embodiments, the formulations of the disclosure, comprise a preservative that is any one or more of Vitamin A, Vitamin C, Vitamin E, retinyl palmitate, methionine, BHA (butylatedhydroxyanisole), BHT (Butylatedhydroxytoulene), selenium, cysteine propyl gallate, phenol, parabens including but not limited to ethyl paraben, methyl paraben, propyl paraben, butyl paraben, edta, citric acid, sodium citrate, benzyl alcohol, chlorobutanol, meta cresol, chloro cresol, benzoic acid, sorbic acid, thiomersal, bronopol diols, propylene glycol, benzyl konium chloride, benzethonium chloride, chlorhexidine gluconate and benzoic acid. In some embodiments, the formulations of the disclosure, comprise a preservative that is a combination of benzyl alcohol, chlorhexidine gluconate and benzoic acid. In some embodiments, the formulations of the disclosure, comprise a preservative in an amount of less than 0.12% to less than 1% of the total weight of the formulation.

In some embodiments, the formulations of the disclosure, comprise benzyl alcohol in an amount of between less than 0.12% to less than 1% of the total weight of the formulation. In some embodiments, the formulations of the disclosure, comprise benzyl alcohol in an amount of less than 1% of the total weight of the formulation. In some embodiments, the formulations of the disclosure, comprise benzyl alcohol in an amount of between less than 1%, 0.5%, 0.4%, 0.3% and 0.2%, of the total weight of the formulation, including the end points.

In some embodiments, the formulations of the disclosure, comprise chlorhexidine gluconate in an amount of between less than 0.12% to less than 1% of the total weight of the formulation. In some embodiments, the formulations of the disclosure, comprise chlorhexidine gluconate in an amount of less than 0.12% of the total weight of the formulation. In some embodiments, the formulations of the disclosure, comprise chlorhexidine gluconate in an amount of between less than 0.12%, 0.11%, 0.10%, 0.09%, 0.08%, 0.07%, 0.06%, 0.05%, 0.04%, 0.03% and 0.02%, of the total weight of the formulation, including the end points.

In some embodiments, the formulations of the disclosure, comprise benzoic acid in an amount of between less than 0.12% to less than 1% of the total weight of the formulation. In some embodiments, the formulations of the disclosure, comprise benzoic acid in an amount of less than 0.2% of the total weight of the formulation. In some embodiments, the formulations of the disclosure, comprise benzoic acid in an amount of between less than 0.2%, 0.1%, 0.09%, 0.08%, 0.07%, 0.06%, 0.05%, 0.04%, 0.03% and 0.02%, of the total weight of the formulation, including the end points.

In some embodiments, the formulations of the disclosure, comprise benzyl alcohol in an amount of between 0.1% o 0.2%, 0.2% to 0.3%, 0.3% to 0.4%, 0.4% to 0.5%, 0.5% to 0.6%, 0.6% to 0.7%, 0.7% to 0.8%, 0.8% to 0.9% or 0.95 to 0.99%, of the total weight of the formulation, including the end points. In some embodiments, the formulations of the disclosure, comprise benzoic acid in an amount of between 0.1% o 0.2%, 0.2% to 0.3%, 0.3% to 0.4%, 0.4% to 0.5%, 0.5% to 0.6%, 0.6% to 0.7%, 0.7% to 0.8%, 0.8% to 0.9% or 0.95 to 0.99%, of the total weight of the formulation, including the end points

In some embodiments, the formulation of the disclosure, comprise benzyl alcohol in an amount of less than 1%; Chlorhexidine Gluconate in an amount of less than 0.12%; and benzoic acid in an amount of less than 0.2% of the total weight of the formulation. In some embodiments, the formulation of the disclosure, comprise benzyl alcohol, Chlorhexidine Gluconate and benzoic acid in an amount or percentage weight as depicted in Table 1.

TABLE 1
Ingredient list with function and purpose, of
exemplary formulation comprising niclosamide.

| Ingredient | Function | Purpose | Amount (w/w) |
|---|---|---|---|
| niclosamide | API | inhibits sperm | Minimum dose to |
| | | motility | see effect is: |
| | | | 0.13% (4 mM) |
| | | | Most likely |
| | | | formulation: |
| | | | 1-5% (100 mM = |
| | | | 3.27%) |
| | | | Max: 10% |
| PEG-400 | excipient | humectant, | 0-65% |
| | | lubricant, | |
| | | solvent | |
| sodium chloride | excipient | adjusting | <1% |
| | | osmolality | |
| lactic acid | excipient | pH modulation | <10% |
| citric acid | excipient | pH modulation | <5% |
| monohydrate | | | |
| potassium bitartrate | excipient | pH modulation | <0.5% |
| sodium hydroxide | excipient | pH modulation | <1% |
| hydroxyethyl | excipient | viscosity enhancer | Most likely |
| cellulose | | | formulation: |
| | | | 1-5% (3% ideal) |
| | | | Max: 10% |
| alginic acid | excipient | viscosity enhancer | <5% |
| polycarbophil | excipient | viscosity enhancer | <5% |
| carbopol | excipient | viscosity enhancer | <5% |
| water | excipient | solvent | 10-90% |
| benzyl alcohol | excipient | preservative | <1% |
| chlorhexidine | excipient | preservative | <0.12% |
| gluconate | | | |
| benzoic acid | excipient | preservative | <0.2% |
| | | Total: | 100% |

In some embodiments, the formulation of the disclosure, comprise niclosamide, PEG-400, carbopol-980 and benzyl alcohol in an amount in an amount or percentage weight as depicted in Table 2.

TABLE 2
Ingredient list with function and purpose,
of gel formulation comprising niclosamide.

| Ingredient | Function | Purpose | Amount (w/w) |
|---|---|---|---|
| niclosamide | API | inhibits sperm | Gel formulation: |
| | | motility | 3-5% (153 mM = |
| | | | 5%) |
| PEG-400 | excipient | humectant, | 35%-65% |
| | | lubricant, | |
| | | solvent | |
| sodium chloride | excipient | adjusting | 0.1% to 1% |
| | | osmolality | |
| lactic acid | excipient | pH modulation | 1% to 5% |
| citric acid | excipient | pH modulation | 1% to 5% |
| monohydrate | | | |
| potassium sodium | excipient | pH modulation | 0.1% to 0.5% |
| tartrate | | | |
| sodium hydroxide | excipient | pH modulation | 0.1% to 0.5% |
| carbopol | excipient | viscosity enhancer | 0.1% to 1% |
| water | excipient | solvent | 10% to 60% |
| benzyl alcohol | excipient | preservative | 0.1% to 0.5% |
| | | Total: | 100% |

In some embodiments of the formulations of the disclosure, the formulation comprises, a) niclosamide at a concentration of 3% to 5% by weight of the formulation; b) a humectant, a lubricant or a solvent, wherein the humectant, the lubricant or the solvent comprises an amount of PEG-400 of 35% to 65% by weight of the formulation and an amount of water of 10% to 60% by weight of the formulation; c) an osmolality modulator, wherein the osmolality modulator comprises an amount of sodium chloride of 0.1% to 1% by weight of the formulation; d) at least one pH modulator, wherein the at least one pH modulator comprises an amount of lactic acid of 1% to 5% by weight of the formulation, an amount of citric acid monohydrate of 1% to 5% by weight of the formulation, an amount of potassium sodium tartrate of 0.1% to 0.5% by weight of the formulation, and an amount of sodium hydroxide of 0.1% to 0.5% by weight of the formulation; e) at least one viscosity modulator, wherein the at least one viscosity modulator comprises an amount of carbopol of 0.1% to 1% by weight of the formulation; and f) at least one preservative, wherein the at least one preservative comprises an amount of benzyl alcohol of 0.1% to 0.5% by weight of the formulation.

In some embodiments of the formulations of the disclosure, the formulation comprises, a) niclosamide at a concentration of 3% to 5% by weight of the formulation; b) a humectant, a lubricant or a solvent, wherein the humectant, the lubricant or the solvent comprises an amount of PEG-400 of 35% to 65% by weight of the formulation and an amount of water of 0% to 60% by weight of the formulation; c) an osmolality modulator, wherein the osmolality modulator comprises an amount of sodium chloride of 0.7% by weight of the formulation; d) at least one pH modulator, wherein the at least one pH modulator comprises an amount of lactic acid of 3.5% by weight of the formulation, an amount of citric acid monohydrate of 2% by weight of the formulation, an amount of potassium sodium tartrate of 0.35% by weight of the formulation, and an amount of sodium hydroxide of 0.1% by weight of the formulation; e) at least one viscosity modulator, wherein the at least one viscosity modulator comprises an amount of carbopol of 0.5% by weight of the formulation; and f) at least one preservative, wherein the at least one preservative comprises an amount of benzyl alcohol of 0.2% by weight of the formulation.

In some embodiments of the formulations of the disclosure, the formulation comprises, a) niclosamide at a concentration of 5% by weight of the formulation; b) a humectant, a lubricant or a solvent, wherein the humectant, the lubricant or the solvent comprises an amount of PEG-400 of 65% by weight of the formulation and an amount of water of 22.65% by weight of the formulation; c) an osmolality modulator, wherein the osmolality modulator comprises an amount of sodium chloride of 0.7% by weight of the formulation; d) at least one pH modulator, wherein the at least one pH modulator comprises an amount of lactic acid of 3.5% by weight of the formulation, an amount of citric acid monohydrate of 2% by weight of the formulation, an amount of potassium sodium tartrate of 0.35% by weight of the formulation, and an amount of sodium hydroxide of 0.1% by weight of the formulation; e) at least one viscosity modulator, wherein the at least one viscosity modulator comprises an amount of carbopol of 0.5% by weight of the formulation; and f) at least one preservative, wherein the at least one preservative comprises an amount of benzyl alcohol of 0.2% by weight of the formulation.

In some embodiments of the formulations of the disclosure, the formulation comprises, a) niclosamide at a concentration of 5% by weight of the formulation; b) a humectant, a lubricant or a solvent, wherein the humectant, the lubricant or the solvent comprises an amount of PEG-400 of 35% by weight of the formulation and an amount of water of 52.65% by weight of the formulation; c) an osmolality modulator, wherein the osmolality modulator comprises an amount of sodium chloride of 0.7% by weight of the formulation; d) at least one pH modulator, wherein the at least one pH modulator comprises an amount of lactic acid of 3.5% by weight of the formulation, an amount of citric acid monohydrate of 2% by weight of the formulation, an amount of potassium sodium tartrate of 0.35% by weight of the formulation, and an amount of sodium hydroxide of 0.1% by weight of the formulation; e) at least one viscosity modulator, wherein the at least one viscosity modulator comprises an amount of carbopol of 0.5% by weight of the formulation; and f) at least one preservative, wherein the at least one preservative comprises an amount of benzyl alcohol of 0.2% by weight of the formulation.

In some embodiments of the formulations of the disclosure, the formulation comprises, a) niclosamide at a concentration of 3% by weight of the formulation; b) a humectant, a lubricant or a solvent, wherein the humectant, the lubricant or the solvent comprises an amount of PEG-400 of 65% by weight of the formulation and an amount of water of 24.65% by weight of the formulation; c) an osmolality modulator, wherein the osmolality modulator comprises an amount of sodium chloride of 0.7% by weight of the formulation; d) at least one pH modulator, wherein the at least one pH modulator comprises an amount of lactic acid of 3.5% by weight of the formulation, an amount of citric acid monohydrate of 2% by weight of the formulation, an amount of potassium sodium tartrate of 0.35% by weight of the formulation, and an amount of sodium hydroxide of 0.1% by weight of the formulation; e) at least one viscosity modulator, wherein the at least one viscosity modulator comprises an amount of carbopol of 0.5% by weight of the formulation; and f) at least one preservative, wherein the at least one preservative comprises an amount of benzyl alcohol of 0.2% by weight of the formulation.

In some embodiments of the formulations of the disclosure, the formulation comprises, a) niclosamide at a concentration of 3% by weight of the formulation; b) a humectant, a lubricant or a solvent, wherein the humectant, the lubricant or the solvent comprises an amount of PEG-400 of 35% by weight of the formulation and an amount of water of 54.65% by weight of the formulation; c) an osmolality modulator, wherein the osmolality modulator comprises an amount of sodium chloride of 0.7% by weight of the formulation; d) at least one pH modulator, wherein the at least one pH modulator comprises an amount of lactic acid of 3.5% by weight of the formulation, an amount of citric acid monohydrate of 2% by weight of the formulation, an amount of potassium sodium tartrate of 0.35% by weight of the formulation, and an amount of sodium hydroxide of 0.1% by weight of the formulation; e) at least one viscosity modulator, wherein the at least one viscosity modulator comprises an amount of carbopol of 0.5% by weight of the formulation; and f) at least one preservative, wherein the at least one preservative comprises an amount of benzyl alcohol of 0.2% by weight of the formulation.

In some embodiments, the formulations of the disclosure, comprise: a) niclosamide at a concentration of 100 mM by weight of the formulation: b) a humectant, a lubricant or a solvent, wherein the humectant, the lubricant or the solvent comprises an amount of PEG-400 of between 0.1% and 65% by weight of the formulation and an amount of water of between 10% and 90% by weight of the formulation; c) an osmolality modulator, wherein the osmolality modulator comprises an amount of sodium chloride of less than 1% by weight of the formulation; d) at least one pH modulator, wherein the at least one pH modulator comprises an amount of lactic acid of less than 10% by weight of the formulation, an amount of citric acid monohydrate of less than 5% by weight of the formulation, an amount of potassium bitartrate of less than 0.5% by weight of the formulation, and an amount of sodium hydroxide of less than 1% by weight of the formulation; e) at least one viscosity modulator, wherein the at least one viscosity modulator comprises an amount of hydroxyethyl cellulose of 3% by weight of the formulation, an amount of alginic acid of less than 5% by weight of the formulation, an amount of polycarbophil of less than 5% by weight of the formulation and an amount of carbopol of less than 5% by weight of the formulation; and f) at least one preservative, wherein the at least one preservative comprises an amount of benzyl alcohol of less than 1% by weight of the formulation, an amount of chlorhexidine gluconate of less than 0.12% by weight of the formulation, and an amount of benzoic acid of less than 0.2% by weight of the formulation.

In some embodiments, the formulation including those in which the formulations of the disclosure, including a semi-solid form, a gel, a lotion or a cream, comprise the ingredients as depicted in Tables 1 and 2 in the amount or percentage by weight, as depicted in Tables 1 and 2.

In some embodiments, the formulation of the disclosure, can be provided in an amount of between 0.1 mL and 1 mL, 0.1 mL and 0.5 mL, 0.5 mL and 1 mL, 1 mL and 1.5 mL, 1.5 mL and 2 mL, 2 mL and 3 mL, 3 mL and 4 mL, 4 mL and 5 mL, 5 mL and 6 mL, 6 mL and 7 mL, 7 mL and 8 mL, 8 mL and 9 mL, 9 mL and 10 mL, 10 mL and 20 mL, 20 mL and 30 mL, 30 mL and 40 mL, 40 mL and 50 mL, 50 mL and 60 mL, 60 mL and 70 mL, 70 mL and 80 mL, 80 mL and 90 mL, 90 mL and 100 mL, 100 mL and 200 mL, 200 mL and 300 mL, 400 mL and 500 mL, 500 mL and 600 mL, 600 mL and 700 mL, 800 mL and 900 mL, and 900 mL and 1000 mL, inclusive of the endpoints. In some embodiments, the formulation of the disclosure, can be provided in an amount of between 0.5 mL and 10 mL, inclusive of the endpoints.

In some embodiments, the formulations of the disclosure, are suitable for intravaginal application by an individual who is not a medical professional. In some embodiments, the formulations of the disclosure are suitable for topical administration by the subject. In some embodiments, the formulations of the disclosure are suitable for direct intravaginal application. In some embodiments, the formulations of the disclosure are suitable for direct intrarectal application.

In some embodiments, the formulations of the disclosure may be administered less than 1 hour before sexual activity. In some embodiments, the formulations of the disclosure may be administered less than 1 minute (min), 2 min, 3 min, 4 min, 5 min, 10 min, 15 min, 20 min, 25 min, 30 min, 35 min, 40 min, 45 min, 50 min, 55 min, 60 min or any number of minutes in between, prior to sexual activity. In some embodiments of the methods of treatment of the disclosure, the female subject is healthy. In some embodiments of the methods of treatment of the disclosure, the semen sample is from a healthy male.

In some embodiments of the methods of treatment of the disclosure, the formulation contacts a sperm cell in vivo or in vitro.

In some embodiments, the formulations of the disclosure are pharmaceutical formulations.

In some embodiments, formulations of the disclosure comprise a semi-solid form. In some embodiments, the semi-solid form comprise one or more of a gel, a hydrogel, a dehydrated gel, a hydrated gel, a paste, an ointment, a cream, and a lotion.

Gels of the disclosure include, but are not limited to, controlled release gels, organogels, extended release gels, amphiphilic gels, hydrophilic gels, non aqueous gels, bioadhesive gels, thermosensitive sol-gel reversible hydrogels, complexation gels and hydrogel.

### Anti-Viral and Anti-Bacterial Formulations

This disclosure provides niclosamide formulations for reducing or inhibiting transmission of infections, including, but not limited to, sexually transmitted infections that cause sexually-transmitted diseases (STDs). niclosamide may be specifically formulated for contraception according to the disclosure and/or formulated for treating or preventing infection. In some embodiments of the disclosure, a subject receives a first niclosamide formulation for contraception and a second niclosamide formulation for treating or preventing infection. In some embodiments, the first and second niclosamide formulations are identical (e.g. same elements in the same proportions or absolute amounts). In some embodiments, the first and second niclosamide formulations are not identical. In some embodiments, the first and second niclosamide formulations are administered to the subject by the same route. In some embodiments, the first and second niclosamide formulations are not administered to the subject by the same route. In some embodiments, the first and the second formulations are the same formulation (a dual-purpose formulation).

Formulations of the disclosure may be “unisex” formulation, because, although contraception has traditionally been focused on female subjects and prevention of disease transmission has traditionally been focused on male subjects, the formulations of the disclosure may be administered to either a male or a female subject with equal safety and efficacy for either promoting contraception and/or treating/preventing infection. In some embodiments, formulations of the disclosure may be administered to a female subject, to reduce or prevent transmission of an infection (or infectious agent) and/or conception. In some embodiments, formulations of the disclosure may be administered to a male subject, to reduce or prevent transmission of an infection (or infectious agent) and/or conception (by his female partner).

In some embodiments, the formulations of this disclosure, are effective in inhibiting transmission of sexually transmitted diseases are provided. In some embodiments, a method includes administering the formulation locally to a potential site of infection or to a potential route of infection (access point to the body or blood stream).

This disclosure provides formulations comprising an effective amount of niclosamide and methods of using the same, for treatment or prevention of an infection in a subject by an infectious agent. In some embodiments, the infectious agent is a virus. In some embodiments, the virus is HSV-2 or HIV. In some embodiments the infectious agent is a bacteria. In some embodiments, the bacteria is M.tb.

In some embodiments, the methods of the disclosure, comprise administering an effective amount of a formulation as disclosed herein, to a subject, for effectively treating or preventing of infection caused by a virus or a bacteria. In some embodiments, the methods of the disclosure, comprise administering an effective amount of a formulation as disclosed herein, in combination with an anti-viral or an anti-bacterial agent or a combination thereof, to a subject, for effectively treating or preventing of infection caused by an infectious virus or a bacteria or both. In some embodiments, the methods of the disclosure, comprise administering a first effective amount of a formulation as disclosed herein, to a subject for inducing contraception, and further comprise administering a second effective amount of a formulation as disclosed herein, to the subject for treating or preventing an infection caused by a virus or a bacteria. In some embodiments, the concentration of niclosamide in the first effective amount of the formulation and the concentration of niclosamide in the second effective amount of the formulation, are different.

In some embodiments of the method of the disclosure, for treatment or prevention of an infection by HSV-2, the second effective amount of the formulation comprises niclosamide at a concentration of between 0.04 μM and 30 μM, between 0.12 μM and 10 μM, and between 0.37 μM and 3.33 μM, inclusive of the endpoints. In some embodiments of the method of the disclosure, for treatment or prevention of an infection by HSV-2, the second effective amount of the formulation comprises niclosamide at a concentration of 0.37 μM. In some embodiments, the formulation of the disclosure, can be used in combination with other anti-viral agents, for treatment and prevention of co-infection by a non-HSV and non-HIV virus. In some embodiments, the formulation of the disclosure, can be used in combination with obatoclax and/or emetine for treatment and prevention of infection by human immunodeficiency virus-2 (HSV-2), HIV-1, enterovirus (EV1), human metapneumovirus (HMPV) and rift valley fever virus (RVFV). In some embodiments, the formulation of the disclosure, can be used in combination with emetine for treatment or prevention of infection by Influenza A virus (FluAV). In some embodiments, the formulation of the disclosure, can be used in combination with brequinar for treatment or prevention of infection by HIV-1 and HIV-2. In some embodiments, the formulation of the disclosure, can be used in combination with suramin for treatment or prevention of infection by HIV-1 and HIV-2. In some embodiments, the formulation of the disclosure, can be used in combination with homoharringtonine for treatment or prevention of infection by EV1.

In some embodiments, the formulation of the disclosure, comprises an effective amount of niclosamide for treatment or prevention of co-infection by HIV-1 and M. tb. In some embodiments, the formulation of the disclosure, for treatment and prevention of infection by M.tb comprises an amount of niclosamide between 2.5 μM and 20 μM, between 2.5 μM and 5 μM, between 5 μM and 10 μM, and between 10 μM and 20 μM, including the end points. In some embodiments, the formulation of the disclosure, for treatment and prevention of infection by HIV-1 comprises an amount of niclosamide between 0.3125 μM and 5 μM, between 0.625 μM and 1.25 μM, and between 1.25 μM and 2.5 μM, including the endpoints. niclosamide inhibits HIV-replication post integration and transcription of integrated HIV-pro virus.

In some embodiments, the formulations of the disclosure, are for use in treating or preventing an infection transduced by a virus or a bacteria, comprising administering an effective amount of the formulation to the subject, wherein, upon contacting an infectious agent, the formulation prevents transmission, infection, replication, survival or growth of the virus or bacteria in the subject. In some embodiments, the formulations of the disclosure, for use in treating or preventing an infection transduced by a virus that communicable by means other than sexual contact.

In some embodiments of the formulations of the disclosure for use in treating or preventing an infection transduced by a virus, the virus is communicated by contacting directly or indirectly an organism infected with the virus or contaminated by the virus.

Directly contacting an organism infected with the virus or contaminated by the virus include but are not limited to directly contacting any part, portion or surface of the organism or contacting any object infected with the virus or contaminated with the virus, surface or material that is or has been in contact with the organism infected with the virus or contaminated with the virus. The organism infected with the virus or contaminated by the virus can present one or more symptom(s) of the infection caused by the virus disease caused by the virus. The symptoms include but are not limited to: 1) fever or hyperthermia, 2) coughing, 3) sneezing, 4) chest congestion, 5) difficulty in breathing or shortness of breath, 6) frequent shivering, 7) muscle pain, 8) headache, 9) loss of taste or smell or both, 10) sore throat, 11) blood discharge in at least one of urine or fecal excretion, mucus, sputum, nasal discharge, urethral discharge, vaginal discharge or skin, 12) loss of weight, 13) loss of appetite, 14) occurrence of rashes or boils or sores or depigmentation or hyperpigmentation of skin, 15) loss of hair, and 14) loss of immunity to infections.

The organism infected with the virus or contaminated by the virus may not present any symptom(s) of the infection caused by the virus disease caused by the virus. The organism infected with the virus or contaminated by the virus is an asymptomatic carrier of the virus. The organism that is an asymptomatic carrier of the virus is a host or a harbor or a reservoir of the virus, wherein the virus survives and replicates in the organism.

The organism infected with the virus or contaminated by the virus can present one or more symptom(s) of the infection caused by the virus disease caused by the virus, is a host or a harbor or a reservoir of the virus, wherein the virus survives and replicates in the organism, and is dissipated or released from the organism into the environment surrounding the organism. The organism that is an asymptomatic carrier of the virus is a host or a harbor or a reservoir of the virus, wherein the virus survives and replicates in the organism, and dissipates from the organism in to the environment surrounding the organism. The virus dissipated or released from the organism infected with the virus or contaminated by the virus, into the environment surrounding the organism, can be transmitted or communicated to a subject that comes in contact with either the environment surrounding the organism. The virus dissipated or released from the organism infected with the virus or contaminated by the virus, into the environment surrounding the organism, can be transmitted or communicated to a subject that comes in direct or indirect contact with the virus in either the environment surrounding the organism or the organism itself.

In some embodiments of the formulations of the disclosure for use in treating or preventing an infection transduced by a virus, the subject contacts the virus by communication of the virus through a fluid media, the fluid media being extracted from, isolated from, secreted by or excreted by, the organism infected with the virus or contaminated by the virus. In some embodiments of the formulations of the disclosure for use in treating or preventing an infection transduced by a virus, the subject contacts the virus by communication of the virus through air exhaled or released by the organism infected with the virus or contaminated by the virus. In some embodiments of the formulations of the disclosure for use in treating or preventing an infection transduced by a virus, the fluid media being extracted from, isolated from, secreted by or excreted by, the organism infected with the virus or contaminated by the virus, comprises a bodily fluid or particulate thereof from the infected or contaminated organism. In some embodiments of the formulations of the disclosure for use in treating or preventing an infection transduced by a virus, the air exhaled or released by the organism infected with the virus or contaminated by the virus, comprises a bodily fluid or particulate thereof from the infected or contaminated organism. In some embodiments of the formulations of the disclosure for use in treating or preventing an infection transduced by a virus, the body fluid comprises exhaled or excreted droplets. In some embodiments of the formulations of the disclosure for use in treating or preventing an infection transduced by a virus, the body fluid comprises aerosolized droplets. In some embodiments of the formulations of the disclosure for use in treating or preventing an infection transduced by a virus, the droplets are between 10⁻⁴ to 10¹ μM in diameter or size. In some embodiments of the formulations of the disclosure for use in treating or preventing an infection transduced by a virus, the bodily fluid comprises sputum, saliva, blood, plasma, serum, lymph fluid, tears, sweat, urine or feces.

In some embodiments of the formulations of the disclosure for use in treating or preventing an infection transduced by a virus, the virus is communicated to the subject from across a physical distance of between 0.1 and 12 feet from the organism. In some embodiments of the formulations of the disclosure for use in treating or preventing an infection transduced by a virus, the virus is communicated to the subject from across a physical distance of 6 feet or less from the organism. In some embodiments of the formulations of the disclosure for use in treating or preventing an infection transduced by a virus, the virus is communicated to the subject from across a physical distance of less than 3 feet from the organism. In some embodiments of the formulations of the disclosure for use in treating or preventing an infection transduced by a virus, the virus is communicated to the subject from across a physical distance of less than 1 feet from the organism.

In some embodiments of the formulations of the disclosure for use in treating or preventing an infection transduced by a virus, the virus survives, remains viable or retains an infectious activity on a surface for between 0.1 minute and 6 months. In some embodiments, the virus survives, remains viable or retains an infectious activity on a surface for between 0.1 minute and 5 minutes. In some embodiments of the formulations of the disclosure for use in treating or preventing an infection transduced by a virus, the virus survives, remains viable or retains an infectious activity on a surface for between 5 minute and 15 minutes. In some embodiments of the formulations of the disclosure for use in treating or preventing an infection transduced by a virus, the virus survives, remains viable or retains an infectious activity on a surface for between 15 minute and 30 minutes. In some embodiments of the formulations of the disclosure for use in treating or preventing an infection transduced by a virus, the virus survives, remains viable or retains an infectious activity on a surface for between 1 hours and 3 hours. In some embodiments, the virus survives, remains viable or retains an infectious activity on a surface for between 24 hours and 72 hours. In some embodiments of the formulations of the disclosure for use in treating or preventing an infection transduced by a virus, the virus survives, remains viable or retains an infectious activity on a surface for between 1 day and 5 days. In some embodiments of the formulations of the disclosure for use in treating or preventing an infection transduced by a virus, the virus survives, remains viable or retains an infectious activity on a surface for between 1 week and 3 weeks. In some embodiments of the formulations of the disclosure for use in treating or preventing an infection transduced by a virus, the virus survives, remains viable or retains an infectious activity on a surface for between 1 month and 6 months. In some embodiments of the formulations of the disclosure for use in treating or preventing an infection transduced by a virus, the virus is the exhaled or excreted droplets or the aerosolized droplets remains viable or retains an infectious activity when floating in air for between 1 hour and 3 hours. In some embodiments of the formulations of the disclosure for use in treating or preventing an infection transduced by a virus, the virus in the exhaled or excreted droplets or the aerosolized droplets remains viable or retains an infectious activity when floating in air for between 1 hour and 6 hours. In some embodiments of the formulations of the disclosure for use in treating or preventing an infection transduced by a virus, the virus in the exhaled or excreted droplets or the aerosolized droplets remains viable or retains an infectious activity when floating in air for up to 1 hour. In some embodiments of the formulations of the disclosure for use in treating or preventing an infection transduced by a virus, the virus in exhaled, excreted or aerosolized droplets of size >5 μM remain suspended in air for longer time than virus in exhaled, excreted or aerosolized droplets of size <5 μM. In some embodiments of the formulations of the disclosure for use in treating or preventing an infection transduced by a virus, the bodily fluid or particulate thereof from the infected or contaminated organism, comprises droplets of varying sizes, e.g. >5 μM and <5 μM. In some embodiments of the formulations of the disclosure for use in treating or preventing an infection transduced by a virus, the survival of the virus in the exhaled or excreted droplets or the aerosolized droplets depends on the parameters of the air including but not limited to air temperature, air humidity, air pressure, air velocity, solar intensity or a combination thereof.

In some embodiments of the formulations of the disclosure for use in treating or preventing an infection transduced by a virus, the virus remains viable or retains an infectious activity when deposited on a surface. In some embodiments of the formulations of the disclosure for use in treating or preventing an infection transduced by a virus, the virus remains viable or retains an infectious activity on a surface, the surface comprises a biological surface. In some embodiments of the formulations of the disclosure for use in treating or preventing an infection transduced by a virus, the surface comprises one or more of a plant, a tree, a crop or a component thereof, skin, hair and nails. In some embodiments of the formulations of the disclosure for use in treating or preventing an infection transduced by a virus, the surface comprises one or more of silk, cotton, cellulose, cork, wool, wood, cardboard, latex, rubber and paper. In some embodiments of the formulations of the disclosure for use in treating or preventing an infection transduced by a virus, the surface does not comprise a biological surface. In some embodiments of the formulations of the disclosure for use in treating or preventing an infection transduced by a virus, the surface comprises an organic surface.

In some embodiments of the formulations of the disclosure for use in treating or preventing an infection transduced by a virus, the surface comprises one or more of carbon fiber, a plastic and synthetic fiber. In some embodiments of the formulations of the disclosure for use in treating or preventing an infection transduced by a virus, the surface comprises an inorganic surface. In some embodiments of the formulations of the disclosure for use in treating or preventing an infection transduced by a virus, the surface comprises one or more of a metal, silicone and glass. In some embodiments of the formulations of the disclosure for use in treating or preventing an infection transduced by a virus, the virus remains viable or retains an infectious activity when deposited on a surface, the surface being clothing, apparel, accessory, ornament, furniture, tool or food item that has been contacted direct or indirect contact with the organism that is infected by the virus or contaminated by the virus.

In some embodiments of the formulations of the disclosure for use in treating or preventing an infection transduced by a virus, the virus does not remain viable or retains an infectious activity when deposited on a surface if the surface is contacted with a disinfectant solution. In some embodiments of the formulations of the disclosure for use in treating or preventing an infection transduced by a virus, the disinfectant is any one of a 70% ethanol solution, a bleaching solution or any commercially available disinfectant solution or a combination thereof. In some embodiments of the formulations of the disclosure for use in treating or preventing an infection transduced by a virus, the virus does not remain viable or retains an infectious activity when deposited on a surface if the surface is contacted with ultraviolet (UV) light. In some embodiments of the formulations of the disclosure for use in treating or preventing an infection transduced by a virus, the subject can be prevented from contacting the virus on a surface or from the environment surrounding an organism infected with a virus or contaminated by a virus, by creating a barrier between the subject and the surface or the organism. In some embodiments of the formulations of the disclosure for use in treating or preventing an infection transduced by a virus, the barrier is a disposable National Institute of Occupational Safety and Health (NIOSH) approved N-95 filtering facepiece respirator, a ANSI/AAMI PB70 highest barrier level gown, surgical gloves, or any personal protective equipment (PPE).

In some embodiments of the formulations of the disclosure for use in treating or preventing an infection transduced by a virus, the organism infected with the virus or contaminated with the virus is a human. In some embodiments of the formulations of the disclosure for use in treating or preventing an infection transduced by a virus, the organism infected with the virus or contaminated with the virus is not a human. In some embodiments of the formulations of the disclosure for use in treating or preventing an infection transduced by a virus, the organism is a mammalian, an avian, a reptilian, an amphibian, a crustacean, an arthropod or a chordata organism. In some embodiments of the formulations of the disclosure for use in treating or preventing an infection transduced by a virus, the virus is a zoonotic virus. In some embodiments of the formulations of the disclosure for use in treating or preventing an infection transduced by a virus, contacting comprises consumption or handling of the organism by the subject. In some embodiments, the organism is a domesticated animal. In some embodiments of the formulations of the disclosure for use in treating or preventing an infection transduced by a virus, the domesticated animal is a pet or an ornamental animal. In some embodiments of the formulations of the disclosure for use in treating or preventing an infection transduced by a virus, the pet or ornamental animal is a dog, a cat, a bird, a reptile, a rodent including but not limited to a mouse, a rat, a rabbit, a hare and a hamster. In some embodiments of the formulations of the disclosure for use in treating or preventing an infection transduced by a virus, the organism is a live-stock animal. In some embodiments of the formulations of the disclosure for use in treating or preventing an infection transduced by a virus, the organism is a dog, a cat, a hamster, a rodent, a poultry including but not limited to hen, duck, goose, turkey and quail, a cattle including but not limited to cow, sheep and goat, a horse, a donkey, a mule, a llama, a camel, pig. In some embodiments, the organism is an aquatic animal including but not limited to a fish, a whale, a squid, an octopus, a crustacean, a reptile, or an amphibian.

In some embodiments of the formulations of the disclosure for use in treating or preventing an infection transduced by a virus, the organism is a wild animal. In some embodiments, the wild animal is a bat, a cat including but not limited to a lion, a tiger, a panther or a jaguar, a fox, a wolf, a coyote, a civet, a snake, a lizard, an amphibian, a rodent, a non-human primate or an ape species.

In some embodiments of the formulations of the disclosure for use in treating or preventing an infection transduced by a virus, the contacting occurs within a distance of 5 miles or less of: i) a human dwelling, ii) a laboratory or a research facility, iii) a market, store, or retail location, iv) a zoo, game reserve, wildlife reserve, land managed for wildlife protection or wildlife sanctuary, v) a farm, a field, or an agricultural location, vi) a hotel, a lodge, a resort or a site for an ecotourism activity, vii) a source of water, a well or a barrel maintained for drinking water, a stream, a river, a lake and an ocean, and/or viii) an airplane, a ship, a boat, a bus, a train, a car, a truck, or any other means of public, commercial or personal transportation. In some embodiments of the formulations of the disclosure for use in treating or preventing an infection transduced by a virus, the communication of the virus by the subject involves direct and indirect contact as follows i) wild animals in and around human dwellings, ii) wild animals hunted, iii) wild animals consumed, iv) wild animals kept as pets, v) wild animals housed in laboratories, vi) wild animals sold in markets, vii) wild animals kept in zoos and sanctuaries, viii) wild animal exposure during agricultural activities, ix) wild animal exposure during ecotourism activities, x) wild animal exposure during wildlife management activities in protected areas, xi) virus exposure in laboratory settings (lab pathogen), and xii) virus exposure via contaminated water. In some embodiments, contacting the organism is intentional. In some embodiments, the organism is personal property, state property, communal property, a hunting target, a food source, a research subject, a pet, a native species, an invasive species, a prey of any one of the foregoing or a predator of any one of the foregoing. In some embodiments, contacting the organism is unintentional. In some embodiments, the organism is a native species, an invasive species or a predator of the subject.

In some embodiments of the formulations of the disclosure for use in treating or preventing an infection transduced by a virus, the virus frequently mutates resulting in generation of strains or serotypes of the virus with altered genomes and surface antigens. In some embodiments of the formulations of the disclosure for use in treating or preventing an infection transduced by a virus, the virus frequently mutates resulting in generation of strains or serotypes of the virus that are not recognized by the immune system of the subject. In some embodiments of the formulations of the disclosure for use in treating or preventing an infection transduced by a virus. In some embodiments, the virus frequently mutates resulting in generation of strains or serotypes of the virus that are resistant to drugs and treatments e.g. anti-retrovirals, antibodies and vaccines.

In some embodiments of the formulations of the disclosure for use in treating or preventing an infection transduced by a virus, the mutation rate of the genome of the virus is between 10-4 and 10-8 mutations per nucleotide per replication cycle. In some embodiments of the formulations of the disclosure for use in treating or preventing an infection transduced by a virus, the mutation rate of the genome of the virus is between 10⁻² and 10⁻⁵ nucleotide substitution per site per year. In some embodiments, the mutation rate of the genome of the virus is 0.80−2.38×10⁻³ nucleotide substitution per site per year. In some embodiments of the formulations of the disclosure for use in treating or preventing an infection transduced by a virus, the mutation rate of the genome of the virus is 1.16−3.30×10⁻³ nucleotide substitution per site per year for non-synonymous substitution and 1.67−4.67×10⁻³ nucleotide substitution per site per year for synonymous substitution.

In some embodiments of the formulations of the disclosure for use in treating or preventing an infection transduced by a virus, the RNA viruses mutate faster than DNA viruses.

In some embodiments of the formulations of the disclosure for use in treating or preventing an infection transduced by a virus, the mutation rate of the genome of the virus depends on the genetic composition or nucleic acid type of the virus and genome size of the virus. In some embodiments of the formulations of the disclosure for use in treating or preventing an infection transduced by a virus, the genome of a virus that is a RNA virus mutates at a higher rate than the genome of a virus that is a DNA virus. In some embodiments of the formulations of the disclosure for use in treating or preventing an infection transduced by a virus, the genome of a virus that is single stranded mutates at a higher rate than the genome of a virus that is double stranded. In some embodiments of the formulations of the disclosure for use in treating or preventing an infection transduced by a virus, the mutation rate of the genome of the virus correlates negatively with the genome size of the virus. In some embodiments of the formulations of the disclosure for use in treating or preventing an infection transduced by a virus, the mutation rates of the genome of the virus is modulated at different levels, including polymerase fidelity, sequence context, template secondary structure, cellular microenvironment, replication mechanisms, proofreading, and access to post-replicative repair. In some embodiments, the mutation rate of the genome of the virus can be influenced by the by virus-encoding diversity-generating elements, as well as by host-encoded cytidine/adenine deaminases.

In some embodiments of the formulations of the disclosure for use in treating or preventing an infection transduced by a virus, the genome of the virus has a medium to high mutation rate, medium mutation rate being >10⁻⁸ mutations per nucleotide per replication cycle and high mutation rate being >10⁻⁶ mutations per nucleotide per replication cycle

In some embodiments of the formulations of the disclosure for use in treating or preventing an infection transduced by a virus, the genome of the virus undergoes high mutation rates and frequent genetic reassortment, resulting in frequent antigenic drift. Antigenic drift is a mechanism for variation by viruses that involves the accumulation of mutations, e.g. point mutations or deletion mutations) within the antibody-binding sites so that the resulting viruses cannot be inhibited well by antibodies against previous strains making it easier for them to spread throughout a partially immune population. Antigenic shift results in different strains of the same virus. In some embodiments of the formulations of the disclosure for use in treating or preventing an infection transduced by a virus, the antigenic drift results in alteration in amino acid sequence and structure of viral proteins. In some embodiments of the formulations of the disclosure for use in treating or preventing an infection transduced by a virus, the antigenic drift results in alteration in the amino acid sequence and structure of viral surface proteins. In some embodiments of the formulations of the disclosure for use in treating or preventing an infection transduced by a virus, the antigenic drift results in alteration in viral glycoprotein proteins Hemagglutinin (H or HA), Neuraminidase (N or NA), or both. In some embodiments of the formulations of the disclosure for use in treating or preventing an infection transduced by a virus, the altered viral glycoprotein proteins Hemagglutinin results in 18 Hemagglutinin variants, H1-H18. In some embodiments of the formulations of the disclosure for use in treating or preventing an infection transduced by a virus, the altered viral glycoprotein proteins Neuraminidase results in 11 Neuraminidase variants, N1-H11. In some embodiments of the formulations of the disclosure for use in treating or preventing an infection transduced by a virus, the antigenic drift results in alteration in viral glycoprotein proteins Hemagglutinin (H or HA), Neuraminidase (N or NA), or both, resulting in viral strains comprising a combination of any one of the Hemagglutinin variants H1-18 and any one of the Neuraminidase variants N1-11.

In some embodiments of the formulations for use in treating or preventing an infection, a genomic sequence of the virus undergoes reassortment. Reassortment is the process by which viruses with a segmented genome, like Influenza virus, swap gene segments. This genetic exchange is possible due to the segmented nature of the viral genome and occurs when two differing viruses e.g. Influenza viruses co-infect a cell. The viral diversity generated through reassortment is vast and plays an important role in the evolution of viruses like Influenza viruses. For example, if two different influenza virus infect a cell, the individual RNA segments of each Influenza virus enter the nucleus. RNAs of both viruses are copied in the nucleus and exported to the cytoplasm, and then are incorporated into new virus particles which bud from the cell. When new virus particles are assembled at the plasma membrane, each of the RNA segments may originate from either infecting virus. Viral progeny that inherit RNAs from both infecting parent viruses are called reassortants.

In some embodiments of the formulations for use in treating or preventing an infection, the genomic sequence of the virus mutates at a rate higher than 10⁻⁶ mutations per nucleotide of the genome per replication cycle.

In some embodiments of the formulations for use in treating or preventing an infection, the genomic sequence of the virus undergoes reassortment at a frequency of 6 to 20%. In some embodiments of the formulations for use in treating or preventing an infection, the genomic sequence of the virus undergoes reassortment at a frequency of 10 to 20%. In some embodiments of the formulations for use in treating or preventing an infection, the genomic sequence of the virus undergoes reassortment more frequently than 20%. In some embodiments of the formulations for use in treating or preventing an infection, the genomic sequence of the virus undergoes reassortment more frequently than 40%.

The time before the symptoms of a viral infection appear is called the incubation period. During this time, viral genomes are replicating and the host is responding, producing cytokines such as interferon that can have global effects, leading to the classical symptoms of an acute infection (e.g., fever, malaise, aches, pains, and nausea). These symptoms are called the prodrome, to distinguish them from those characteristic of infection (e.g. paralysis for poliovirus, hemorrhagic fever for Ebolaviruses, rash for measles virus). Whether or not an infected person is contagious (i.e. is shedding virus) during the incubation period depends on the virus. In some embodiments, the subject once infected or transmitted with the virus is contagious (i.e. shedding virus) during the incubation period. In some embodiments, the subject once infected or transmitted with the virus is not contagious (i.e. shedding virus) during the incubation period.

In some embodiments, the virus has an incubation period of 1 day to more than 1 year. In some embodiments, the virus has an incubation period of 1 week to more than 4 weeks. In some embodiments, the virus has an incubation period of 1 week to 4 weeks. In some embodiments, the virus has an incubation period of 1 to 21 days. In some embodiments, the virus has an incubation period of 1 to 15 days. In some embodiments, the virus has an incubation period of 0.5 to 15 days. In some embodiments, the virus has an incubation period of 0.5 to 1.5 days. In some embodiments, the virus has an incubation period of 5 to 15 days. The basic reproduction number or reproduction number (R0), also called the basic reproduction ratio or rate or the basic reproductive rate, is a metric used to describe the contagiousness or transmissibility of infectious agents, including a virus. R0 provides some information regarding the speed at which a disease is capable of spreading in a specific population. R0 is the number of secondary cases of infection that would result from a case in a specific population. An R0 for an infectious disease event is generally reported as a single numeric value or low-high range, and the interpretation is that a disease outbreak is expected to continue if R0 has a value >1, the disease will be stable if R0 has a value=1, and the disease will end if R0 is <1. The magnitude of the R0 value for a disease event can be used to determine the potential size of an outbreak or epidemic often is based on, and to estimate the proportion of the population that must be vaccinated to eliminate an infection from that population. In some embodiments of the formulations of the disclosure, the virus has a reproduction number (R0) between 0.9 and 18. In some embodiments, the virus has a R0 between 12 and 18. In some embodiments, the virus has a R0 between 1.5 and 2.5. In some embodiments, the virus has a R0 between 1.5 and 3.5. In some embodiments, the virus has a R0 between 2 to 5. In some embodiments, the virus has a R0 between 5 and 6.

In some embodiments of the formulations for use in treating or preventing an infection, the virus is an RNA virus.

In some embodiments of the formulations for use in treating or preventing an infection, the RNA virus is a positive-strand RNA virus. In some embodiments of the formulations for use in treating or preventing an infection, the positive-strand RNA virus belongs to the family of Picornaviridae, Astroviridae, Caliciviridae, Hepeviridae Flaviviridae, Togaviridae, Arteriviridae, or Coronaviridae.

In some embodiments of the formulations for use in treating or preventing an infection, the positive-strand RNA virus belongs to the family of Coronaviridae. In some embodiments of the formulations for use in treating or preventing an infection, the Coronaviridae virus is Bat coronavirus CDPHE15, Bat coronavirus HKU10, Rhinolophus ferrumequinum alphacoronavirus HuB-2013, Human coronavirus 229E, Lucheng Rn rat coronavirus, Ferret coronavirus, Mink coronavirus 1, Miniopterus bat coronavirus 1, Miniopterus bat coronavirus HKU8, Myotis ricketti alphacoronavirus Sax-2011, Nyctalus velutinus alphacoronavirus SC-2013, Porcine epidemic diarrhea virus, Scotophilus bat coronavirus 512, Rhinolophus bat coronavirus HKU2, Human coronavirus NL63, NL63-related bat coronavirus strain BtKYNL63-9b, Human coronavirus OC43, China Rattus coronavirus HKU24, Human coronavirus HKU1, Murine coronavirus—type species, Bat Hp-betacoronavirus Zhejiang2013, Hedgehog coronavirus 1, Middle East respiratory syndrome-related coronavirus (MERS-CoV), Pipistrellus bat coronavirus HKU5, Tylonycteris bat coronavirus HKU4, Rousettus bat coronavirus GCCDC1, Rousettus bat coronavirus HKU9, Severe acute respiratory syndrome-related coronavirus, Wigeon coronavirus HKU20, Bulbul coronavirus HKU11—type species, Porcine coronavirus HKU15, Munia coronavirus HKU13, White-eye coronavirus HKU16, Night heron coronavirus HKU19, Common moorhen coronavirus HKU21, Beluga whale coronavirus SW1 or Avian coronavirus—type species.

Coronaviridae virus or coronavirus is a large and a diverse group of positively-stranded RNA viruses with a nucleic acid size of 25,000 to 33,000 nucleotides. Coronavirus are known to cause a variety of pathological conditions in both humans and non-human animals. Coronavirus is composed of an envelope and a helical nucleocapsid with club-shaped surface projections that provide “attachment to cells, hemagglutination, and membrane fusion. Coronavirinae are divided into four genera: alpha-, beta-, gamma-, and delta-coronavirus.

In some embodiments of the formulations for use in treating or preventing an infection, the Coronaviridae virus is an alpha-coronaviridae virus. In some embodiments of the formulations for use in treating or preventing an infection, the alpha-coronaviridae virus is Bat coronavirus HKU10, Human coronavirus 229E, Human coronavirus NL63, Miniopterus bat coronavirus 1, Miniopterus bat coronavirus HKU8, Mink coronavirus 1, Porcine epidemic diarrhoea virus, Rhinolophus bat coronavirus HKU2, Scotophilus bat coronavirus 512. In some embodiments of the formulations for use in treating or preventing an infection, the Coronaviridae virus is a beta-coronaviridae virus. In some embodiments of the formulations for use in treating or preventing an infection, the beta-coronaviridae virus is Betacoronavirus 1, Hedgehog coronavirus 1, Human coronavirus HKU1, Middle East respiratory syndrome-related coronavirus, Murine coronavirus, Pipistrellus bat coronavirus HKU5, Rousettus bat coronavirus HKU9, Severe acute respiratory syndrome-related coronavirus, Tylonycteris bat coronavirus HKU4. In some embodiments of the formulations for use in treating or preventing an infection, the Coronaviridae virus is a delta-coronaviridae virus. In some embodiments of the formulations for use in treating or preventing an infection, the delta-coronaviridae virus is Bulbul coronavirus HKU11, Common moorhen coronavirus HKU21, Coronavirus HKU15, Munia coronavirus HKU13, Night heron coronavirus HKU19, Thrush coronavirus HKU12, White-eye coronavirus HKU16, Wigeon coronavirus HKU20.

In some embodiments of the formulations for use in treating or preventing an infection, the Coronaviridae virus is a delta-coronaviridae virus. In some embodiments of the formulations for use in treating or preventing an infection, the delta-coronaviridae virus is Avian coronavirus or Beluga whale coronavirus SW1.

In some In some embodiments of the formulations for use in treating or preventing an infection, the Coronaviridae virus is Middle East respiratory syndrome-related coronavirus (MERS-CoV), Severe acute respiratory syndrome coronavirus (SARS-CoV) or Severe acute respiratory syndrome coronavirus 2 (SARS-CoV-2).

In some embodiments of the formulations for use in treating or preventing an infection, the Coronaviridae virus is Severe acute respiratory syndrome coronavirus 2 (SARS-CoV-2). In some embodiments of the formulations for use in treating or preventing an infection, the Severe acute respiratory syndrome coronavirus 2 (SARS-CoV-2) is the causative pathogenic agent for the coronavirus disease 2019 (COVID-19).

In some embodiments of the formulations for use in treating or preventing an infection, the RNA virus is a negative-strand RNA virus. In some embodiments of the formulations for use in treating or preventing an infection, the negative-strand RNA virus belongs to the family of Orthomyxoviridae, Paramyxoviridae, Rhabdoviridiae, Filoviruses, or Arenaviruses.

In some embodiments of the formulations for use in treating or preventing an infection, the negative-strand virus is Influenza virus, Sendai virus, Human parainfluenza virus 1 (hPIV1), Simian virus 5 (SV5, PIV5), Mumps virus, Newcastle disease virus (NDV), Measles virus, Rinderpest virus, Respiratory syncytial virus (RSV), Vesicular stomatitis virus (VSV), Rabies virus, Ebola virus, Marburg virus, Lymphocytic choriomeningitis virus (LCMV), Junin virus, or Lassa fever virus.

In some embodiments of the formulations for use in treating or preventing an infection, the negative-strand virus is Influenza virus. In some embodiments of the formulations for use in treating or preventing an infection, the Influenza virus is an Influenza virus Type A, B, C or D.

In some embodiments of the formulations for use in treating or preventing an infection, the Influenza virus is an Influenza virus Type A. In some embodiments of the formulations for use in treating or preventing an infection, the Influenza virus Type A is an Influenza virus Type A (H3N2). In some embodiments of the formulations for use in treating or preventing an infection, the Influenza virus Type A is an Influenza virus Type A (H1N1).

In some embodiments of the formulations for use in treating or preventing an infection, the Influenza virus is an Influenza virus Type B. In some embodiments of the formulations for use in treating or preventing an infection, the Influenza virus Type B is an Influenza virus Type B (Victoria). In some embodiments of the formulations for use in treating or preventing an infection, the Influenza virus Type B is an Influenza virus Type B (Yamagata).

In some embodiments of the formulations for use in treating or preventing an infection, the virus is a retrovirus. In some embodiments of the formulations for use in treating or preventing an infection, the retrovirus is a Lentivirus, Alpharetrovirus, Betaretrovirus, Deltaretrovirus, Gammaretrovirus or Epsilonretrovirus. In some embodiments of the formulations for use in treating or preventing an infection, the retrovirus is a Simian immunodeficiency virus (SIV), Human immunodeficiency virus-1 (HIV-1), HIV-2, Feline immunodeficiency virus (FIV), Equine infectious anemia virus (EIAV), Mouse mammary tumor-like virus (MMTV), Mason-Pfizer monkey virus (MPMV), Respiratory syncytial virus RSV, bovine leukemia virus (BLV), Human T-cell leukemia virus-1 (HTLV-1), HTLV-2, Murine leukemia virus (MuLV), Gibbon ape leukemia virus (GALV).

In some embodiments of the formulations for use in treating or preventing an infection, the virus is a DNA virus. In some embodiments of the formulations for use in treating or preventing an infection, DNA virus is Adenovirus, infectious canine hepatitis virus, Papillomavirus, polyomaviridae, simian vacuolating virus, Parvovirus B19, canine parvovirus, Herpes simplex virus, varicella-zoster virus, cytomegalovirus, Epstein-Barr virus, Smallpox virus, cow pox virus, sheep pox virus, monkey pox virus, vaccinia virus, Torque teno virus.

### Pharmaceutical Forms and Components

In some embodiments, the formulation of the disclosure is a pharmaceutical formulation.

In some embodiments, pharmaceutical formulations of the disclosure comprise a solid form. In some embodiments, the solid form of the disclosure may comprise one or more of a particulate, a granule, a powder, a capsule, a tablet, an encapsulated semi-solid, an encapsulated liquid, a non-biodegradable implant, a biodegradable implant (e.g. for releasing the niclosamide), a deformable matrix, a polymer matrix, and a film. In some embodiments, the solid form comprise one or more of a filler, an excipient, an anti-adherent, a coating, a coloring agent, a glidant, a preservative, a sorbent, a bulking agent, a lubricating agent, an anti-oxidant, a binding agent, a disintegration agent, and a buffering agent. In some embodiments, the pharmaceutical formulations of the disclosure in solid form, may be dissolved in a suitable solvent to form a semi-solid formulation, like a gel, a suspension, a cream or a lotion or a paste. In some embodiments, wherein the formulation is provided as a monotherapy, the pharmaceutical formulations of the disclosure does not comprise a dry solid formulation. In some embodiments, wherein the formulation is provided as a monotherapy, the pharmaceutical formulations of the disclosure does not comprise one or more of a pill, a powder, a capsule, a tablet or any combination thereof.

In some embodiments, the pharmaceutical formulation of the disclosure comprise a matrix. In some embodiments, the matrix of the pharmaceutical composition of the disclosure may comprise a material including, but not limited to, hydrophobic matrices (e.g., polyethylene, polyvinyl chloride, ethyl cellulose and acrylate polymers and their copolymers), lipid matrices, hydrophilic matrices, cellulose derivatives (e.g. methylcellulose); hydroxyethylcellulose (e.g. hydroxypropylmethyl cellulose (HPMC)); sodium carboxymethylcellulose; non cellulose natural or semi synthetic polymers (e.g. agaragar); carob gum; alginates; molasses; polysaccharides of mannose and galactose, chitosan and modified starches; polymers of acrylic acid (e.g. carbopol-934), and bio-degradable matrices and mineral matrices.

In some embodiments, pharmaceutical formulations of the disclosure comprise a semi-solid form. In some embodiments, the semi-solid form comprise one or more of a non-Newtonian fluid, a gel, a hydrogel, a dehydrated gel, a hydrated gel, a paste, an ointment, a cream, a lotion, and a coating. In some embodiments, the semi-solid form comprise one or more of a filler, an excipient, an anti-adherent, a coating, a coloring agent, a glidant, a preservative, a sorbent, a bulking agent, a lubricating agent, an anti-oxidant, a vehicle (e.g. a polymeric matrix within the semi-solid form), a binding agent, a disintegration agent, a buffering agent, a solvent, a viscosity agent, and a stability agent.

Gels of the disclosure include, but are not limited to, controlled release gels, organogels, extended release gels, amphiphilic gels, hydrophilic gels, non aqueous gels, bioadhesive gels, thermosensitive sol-gel reversible hydrogels, complexation gels and hydrogel.

In some embodiments the pharmaceutical formulation of the disclosure comprise a liquid form. In some embodiments, the liquid form comprise one or more of a drop, a solution, a flowing fluid, an aerosolized fluid, a suspension, and an emulsion. In some embodiments, the liquid form comprise one or more of an excipient, a buffering agent, a bulking agent, a lubricating agent, an anti-oxidant, a preservative, a coloring agent, a solvent, a viscosity agent, and a stability agent.

Excipients of the disclosure include, but are not limited to, inorganic chemicals (e.g., calcium phosphates, calcium carbonate, calcium sulfate, halites, metallic oxides), organic chemicals (e.g., carbohydrates, sugars, actual sugars, sugar alcohols, artificial sweeteners), starch (e.g., modified starch, dried starch, converted starch), cellulose (e.g., cellulose ethers, cellulose esters, CMC and croscarmellose sodium, microcrystalline cellulose), petrochemicals, glycols (e.g., polyethylene glycol and propylene glycol), povidones, mineral hydrocarbons (petrolatum, mineral waxes, and mineral oils), acrylic polymers, some petrochemical excipients, oleochemicals (e.g., fatty alcohols, mineral 7 stearates, glycerin), some oleochemical excipients and proteins.

Binding agents of the disclosure include, but is not limited to, saccharides and their derivatives (e.g., disaccharides: sucrose, lactose), polysaccharides and their derivatives (e.g., starches, cellulose or modified cellulose such as microcrystalline cellulose and cellulose ethers such as hydroxypropyl cellulose (HPC)), sugar alcohols (e.g., xylitol, sorbitol or maltitol), protein gelatin, and synthetic polymers (e.g., polyvinylpyrrolidone (PVP), polyethylene glycol (PEG)).

Disintegration agents of the disclosure include, but is not limited to, crosslinked polymers (e.g., cross-linked polyvinylpyrrolidone (crospovidone), cross-linked sodium carboxymethyl cellulose (croscarmellose sodium) and modified starch sodium starch glycolate.

Fillers of the disclosure include, but is not limited to, plant cellulose, dibasic calcium phosphate, vegetable fats and oils, lactose, sucrose, glucose, mannitol, sorbitol, calcium carbonate, and magnesium stearate.

Preservatives of the disclosure include, but is not limited to, antioxidants (e.g. vitamin A, vitamin E, vitamin C, retinyl palmitate, and selenium), amino acids (cysteine and methionine), and citric acid, sodium citrate and synthetic preservatives including parabens (e.g., methyl paraben and propyl paraben).

Pharmaceutical formulations of the disclosure may comprise suitable carriers, buffers, excipients, and agents that are incorporated into formulations to provide improved transfer, delivery, tolerance, and the like. A multitude of appropriate formulations can be found in the formulary known to all pharmaceutical chemists: Remington's Pharmaceutical Sciences (15th Ed, Mack Publishing Company, Easton, Pa. (1975)), particularly Chapter 87 by Blaug, Seymour, therein. These formulations include, for example, powders, pastes, ointments, jellies, waxes, oils, lipids, lipid (cationic or anionic) containing vesicles (such as Lipofectin™), DNA conjugates, anhydrous absorption pastes, oil-in-water and water-in-oil emulsions, emulsions carbowax (polyethylene glycols of various molecular weights), semi-solid gels, and semi-solid mixtures containing carbowax. Any of the foregoing mixtures may be appropriate in treatments and therapies in accordance with the disclosure, provided that the active ingredient in the formulation is not inactivated by the formulation and the formulation is physiologically compatible and tolerable with the route of administration. See also Baldrick P. “Pharmaceutical excipient development: the need for preclinical guidance.” Regul. Toxicol Pharmacol. 32(2):210-8 (2000), Wang W. “Lyophilization and development of solid protein pharmaceuticals.” Int. J. Pharm. 203(1-2):1-60 (2000), Charman W N “Lipids, lipophilic drugs, and oral drug delivery-some emerging concepts.” J Pharm Sci. 89(8):967-78 (2000), Powell et al. “Compendium of excipients for parenteral formulations” PDA J Pharm Sci Technol. 52:238-311 (1998) and the citations therein for additional information related to formulations, excipients and carriers well known to pharmaceutical chemists.

### Pharmaceutically Acceptable Carriers

niclosamide may be combined with a pharmaceutically-acceptable carrier to produce formulations or compositions suitable for therapeutic administration in vivo, in vitro or ex vivo. The term “pharmaceutically acceptable carrier” includes, but is not limited to, any and all solvents, dispersion media, coatings, antibacterial and antifungal agents, isotonic and absorption delaying agents, and the like, compatible with pharmaceutical administration. Suitable carriers are described in the most recent edition of Remington's Pharmaceutical Sciences, a standard reference text in the field, which is incorporated herein by reference. Preferred examples of such carriers or diluents include, but are not limited to, water, saline, ringer's solutions, dextrose solution, and 5% human serum albumin. Liposomes and non-aqueous vehicles such as fixed oils may also be used. The use of such media and agents for pharmaceutically active substances is well known in the art. Except insofar as any conventional media or agent is incompatible with the active compound, use thereof in the compositions is contemplated. Supplementary active compounds can also be incorporated into the compositions.

A formulation of the disclosure is formulated to be compatible with its intended route of administration. In some embodiments, a formulation of the disclosure comprises a pharmaceutically-acceptable carrier that is compatible with the intended route of administration of the formulation. In some embodiments, a formulation of the disclosure comprises a pharmaceutically-acceptable carrier that is compatible with topical or intravaginal administration of the formulation. In some embodiments, a formulation of the disclosure further comprise an additional compound or agent to render the formulation compatible with its intended route of administration. Examples of routes of administration include, but are not limited to, parenteral, e.g., intradermal, subcutaneous, transdermal (i.e., topical), transmucosal, intravaginal, and rectal administration. Formulations or the pharmaceutically-acceptable carriers therein used for parenteral, intradermal, or subcutaneous application may include the following components: a sterile diluent such as water for injection, saline solution, fixed oils, polyethylene glycols, glycerin, propylene glycol or other synthetic solvents; antibacterial agents such as benzyl alcohol or methyl parabens; antioxidants such as ascorbic acid or sodium bisulfite; chelating agents such as ethylenediaminetetraacetic acid (EDTA); buffers such as acetates, citrates or phosphates, and agents for the adjustment of tonicity such as sodium chloride or dextrose. The pH can be adjusted with acids or bases, such as hydrochloric acid or sodium hydroxide. The parenteral preparation can be enclosed in ampoules, disposable syringes or multiple dose vials made of glass or plastic.

The formulations or the pharmaceutically-acceptable carriers therein suitable for injectable use may include sterile aqueous solutions (where water soluble) or dispersions and sterile powders for the extemporaneous preparation of sterile injectable solutions or dispersion. In preferred embodiments, pharmaceutical formulations or the pharmaceutically-acceptable carriers therein are stable under the conditions of manufacture and storage and are preserved against the contaminating action of microorganisms such as bacteria and fungi. The carrier can be a solvent or dispersion medium containing, for example, water, ethanol, polyol (for example, glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In some embodiments, the formulation may include isotonic agents, for example, sugars, polyalcohols such as manitol, sorbitol, sodium chloride in the formulation. Prolonged absorption of the injectable compositions can be brought about by including in the formulation an agent which delays absorption, for example, aluminum monostearate and gelatin.

Sterile injectable pharmaceutical formulations or the pharmaceutically-acceptable carriers therein can be prepared by incorporating the active compound in the required amount in an appropriate solvent with one or a combination of ingredients enumerated above, as required, followed by filtered sterilization. Generally, dispersions are prepared by incorporating the active compound into a sterile vehicle that contains a basic dispersion medium and the required other ingredients from those enumerated above. In the case of sterile powders for the preparation of sterile injectable solutions, methods of preparation are vacuum drying and freeze-drying that yields a powder of the active ingredient plus any additional desired ingredient from a previously sterile-filtered solution thereof.

Systemic administration can also be by a transmucosal or transdermal route. For transmucosal or transdermal administration, penetrants appropriate to the barrier to be permeated are used in the formulation. Such penetrants may include detergents, bile salts, and fusidic acid derivatives. Transmucosal administration can be accomplished through the use of implants and/or suppositories. For transdermal administration, the active compounds may be formulated into ointments, salves, gels, or creams.

In one embodiment, niclosamide are prepared with carriers that will protect the compound against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Liposomal suspensions can also be used as pharmaceutically acceptable carriers.

Pharmaceutical formulations comprising niclosamide may comprise a dosage unit form for ease of administration and uniformity of dosage. Dosage unit form as used herein refers to physically discrete units suited as unitary dosages for the subject to be treated; each unit containing a predetermined quantity of active compound calculated to produce the desired therapeutic effect in association with the required pharmaceutical carrier. The specification for the dosage unit forms of the invention are dictated by and directly dependent on the unique characteristics of the active compound and the particular therapeutic effect to be achieved, and the limitations inherent in the art of compounding such an active compound for the treatment of individuals.

The pharmaceutical compositions can be included in a container, pack, tube, or dispenser together with instructions for administration.

### Sperm Motility Assessment

Sperm motility is a functional measurement of the sperm themselves. Sperm may be sampled directly from semen or from washed sperm samples, and assessed for motility either manually or by CASA (Computer Assisted Sperm Analysis) (Chapter 59—Male Reproductive System. Systems Toxicologic Pathology. Dianne M. Creasy, Robert E. Chapin, in Haschek and Rousseaux's Handbook of Toxicologic Pathology (Third Edition), 2013; Amann R P et al. Computer-assisted sperm analysis (CASA): capabilities and potential developments. Theriogenology. 2014 Jan. 1; 81(1):5-17).

Sperm motility (expressed as %) may be evaluated in duplicate within 1 h of semen collection, by counting moving and nonmoving sperm in several microscopic fields. In some embodiments, at least 200 spermatozoa are counted and classified as (1) “rapidly progressive” (class A) that move forward with speed of at least 25 μm/s (half a tail or 5 head lengths); (2) “slowly progressive” (class B) that move forward with more than 5 μm/s (one head lengths) but less than 25 μm/s; (3) “nonprogressive” (class C) that are slow and only move less than 5 μm/s; and (4) “immotile” (class D) that do not move and appear dead. In preferred embodiments, only complete spermatozoa (head with tail) are included in such counts. Slow-moving sperm (class B+C) can be easily and accurately counted compared with rapidly moving (class A) sperm. A multi-button tally, preferably a digital one, is used to differentially count such motility (for additional detail, see Chapter 23: Standardized semen analysis and quality control management for multicenter male reproductive toxicology clinical trials, Sikka S. C. et al., in Bioenvironmental Issues Affecting Men's Reproductive and Sexual Health, 2018, the contents of which are incorporated herein by reference in their entirety).

### Therapeutically Effective Amounts

A therapeutically effective dose may be a therapeutically effective amount of the formulation of the disclosure, that when contacted with a sperm cell, is sufficient to decrease motility of that cell when compared with a control value. A therapeutically effective dose may be a portion of a therapeutically effective amount. For example, a therapeutically effective dose may be half of the therapeutically effective amount provided in two separate administrations over a period of time, that in aggregate, provide the therapeutically effective amount of the formulation prior to the sexual activity.

A therapeutically effective amount of the formulation of the disclosure relates generally to the amount needed to achieve a therapeutic objective. A “therapeutically effective amount” of the formulation of the disclosure is an amount of the formulation that when contacted with one or more sperm cells, for a sufficient amount of time, induces a decrease or an inhibition of the motility of one or more sperm cell(s), as compared to a control value (e.g. the motility of the sperm cells in a semen sample that has not been contacted with the formulation).

A “sufficient amount of time” of contacting the formulation of the disclosure with a semen sample relates generally to the amount needed to achieve a therapeutic objective. “Sufficient amount of time” of the formulation of the disclosure is an amount of time for which when the formulation, when contacted, with one or more sperm cell(s) induces a decrease or an inhibition of the motility of at least one of the one or more sperm cells.

In some embodiments the decrease or inhibition of the motility of the sperm cell is measured as a percentage reduction in progression speed of the sperm cell. In some embodiments the decrease or inhibition in the motility of the sperm cell is measured as a percentage reduction in linear progression speed of the sperm cell relative to a control value.

In some embodiments, the control value is the speed of a healthy sperm cell or an average speed of a plurality of healthy sperm cells. In some embodiments, the healthy sperm cell(s) have not contacted the formulation.

In some embodiments the therapeutically effective amount of the formulation of the disclosure is an amount that when contacted with one or more sperm cell(s) induces a reduction in sperm motility by at least 50%, 60%, 70%, 80%, 90% or 99% or any percentage in between, in the motility of the one or more sperm cell(s) relative to the control value. In some embodiments the therapeutically effective amount of the formulation of the disclosure is an amount that when contacted with one or more sperm cells induces a complete reduction in number of motile sperm cells (zero motile sperm or 100% immobile sperm cells), for example, in an in vitro test, relative to the number of motile sperm cells as measured before contact with the formulation, or relative to the number of motile sperm cells as measured in a sample that has not contacted the formulation.

In some embodiments a therapeutically effective amount of the formulation of the disclosure is an amount that decreases or reduces the motility of the sperm cell or a portion of the plurality of sperm cells from a speed of more than 25 μm/s (one head lengths), before contacting with the formulation, to a speed of less than 5 μm/s (one head lengths), after contacting with the formulation. In some embodiments a therapeutically effective amount of the formulation of the disclosure is an amount that decreases or reduces the motility of the sperm cell or a portion of the plurality of sperm cells from a speed of more than 25 μm/s (one head lengths), before contacting with the formulation, to a speed of 0 μm/s (one head lengths), after contacting with the formulation. In some embodiments a therapeutically effective amount of the formulation of the disclosure is an amount that decreases or reduces the motility of the sperm cell or a portion of the plurality of sperm cells from a speed of at least 25 μm/s (half a tail or 5 head lengths), before contacting with the formulation to less than 25 μm/s to a speed of after contacting with the formulation. In some embodiments a therapeutically effective amount of the formulation of the disclosure is an amount that decreases or reduces the motility of the sperm cell or a portion of the plurality of sperm cells from a speed of at least 5 μm/s (head lengths), before contacting with the formulation to less than 25 μm/s to a speed of after contacting with the formulation.

In some embodiments the therapeutically effective amount of the formulation of the disclosure is an amount that contacted with a plurality of sperm cells induces an decrease in motility of at least 90%, 95%, 97%, 99%, 100% or any percentage in between of the plurality of sperm cells from at least 25 μm/s (half a tail or 5 head lengths) before contacting with the formulation, to a speed of less than 5 μm/s after contacting with the formulation.

In some embodiments the therapeutically effective amount of the formulation of the disclosure is an amount that contacted with a plurality of sperm cells induces an decreases or reduces the motility of at least 90%, 95%, 97%, 99%, 100% or any percentage in between of the plurality of sperm cells of sperm cells from at least 25 μm/s (half a tail or 5 head lengths) before contacting with the formulation, to a speed of 0 μm/s after contacting with the formulation.

In some embodiments, including those in which the formulations of the disclosure comprise a semi-solid or a liquid form, the formulation may comprise a concentration of the niclosamide, at a concentration between 1 mM and 100 mM, inclusive of the endpoints. In some embodiments, the formulation may comprise a concentration of the niclosamide, at a concentration between 1 mM and 10 mM, 10 mM and 20 mM, 20 mM and 30 mM, 30 mM and 40 mM, 40 mM and 50 mM, 50 mM and 60 mM, 60 mM and 70 mM, 70 mM and 80 mM, 80 mM and 90 mM, 90 mM and 100 mM, inclusive of the endpoints. In some embodiments, the formulation may comprise a concentration of niclosamide at a concentration of 10 mM. In some embodiments, the formulation may comprise niclosamide at a concentration of 50 mM. In some embodiments, the formulation may comprise niclosamide at a concentration of 100 mM.

A “therapeutically effective amount” or “an effective amount” of the formulation of the disclosure also relates the amount needed to achieve complete inhibition or reduction in a viral titer (e.g. of a HIV virus or a HSV-2 virus) in a cell treated with the formulation of the disclosure, as compared to an untreated or control cell (vehicle treated cell). A “therapeutically effective amount” of the formulation of the disclosure also relates the amount needed to achieve inhibition or reduction in replication of a virus (e.g. a HIV virus or a HSV-2 virus) in a cell treated with the formulation of the disclosure, as compared to an untreated or control cell (vehicle treated host cell). In some embodiments, a cell treated with a therapeutically effective amount of the formulation of the disclosure, upon infection with a virus (e.g. HIV-1 or HSV-2), produces a viral titer of at least 50%, 60%, 70%, 80%, 90% or 100% lower than the viral titer produced by an untreated host cell or a control cell.

A “therapeutically effective amount” or “an effective amount” of the formulation of the disclosure is an amount of a niclosamide formulation sufficient to treat or prevent infection by a virus. In some embodiments, the subject receiving a “therapeutically effective amount” or “an effective amount” of the formulation of the disclosure is exposed to a virus (e.g. HIV-1 or HIV-2) but is not infected by the virus (does not present signs or symptoms of infection). In some embodiments, the subject receiving a “therapeutically effective amount” or “an effective amount” of the formulation of the disclosure has been exposed to a virus or infected by a virus (e.g. HIV-1 or HIV-2) but, following administration of the therapeutically effective amount of the formulation, demonstrates a reduction in a severity of a sign or symptom of the infection (including a reduction in a viral titre value from before and after administration of the formulation).

A “therapeutically effective amount” or “an effective amount” of the formulation of the disclosure is an amount of a niclosamide formulation sufficient to treat or prevent infection by a virus or bacteria. A “therapeutically effective amount” or “an effective amount” of the formulation of the disclosure for use in treating or preventing an infection transduced by a virus or bacteria, is an amount of the formulation which upon contacting a virus, prevents transmission, infection, replication, survival or growth of the virus or bacteria.

In some embodiments, the subject receiving a “therapeutically effective amount” or “an effective amount” of the formulation of the disclosure is exposed to a virus (e.g. MERS-CoV or SARS-CoV or SARS-CoV-2) but is not infected by the virus (does not present signs or symptoms of infection). In some embodiments, the subject receiving a “therapeutically effective amount” or “an effective amount” of the formulation of the disclosure has been exposed to a virus or infected by a virus (e.g. MERS-CoV or SARS-CoV or SARS-CoV-2) but, following administration of the therapeutically effective amount of the formulation, demonstrates a reduction in a severity of a sign or symptom of the infection (including a reduction in a viral titre value from before and after administration of the formulation).

A “therapeutically effective amount” or “an effective amount” of the formulation of the disclosure also relates the amount needed to achieve inhibition or reduction in infection by M. tb in cells treated with the formulation of the disclosure, as compared to an untreated or a control cell (e.g., vehicle treated cell). A “therapeutically effective amount” of the formulation of the disclosure also relates the amount needed to achieve inhibition or reduction of growth of M.tb in a cell treated with the formulation of the disclosure, as compared to an untreated or control cell (e.g. vehicle treated host cell), wherein the growth of the M. tb in the cell is calculated in terms of colony forming units of the M.tb. In some embodiments, a cell treated with a therapeutically effective amount of the formulation of the disclosure, upon infection with M.tb, produces at least 50%, 60%, 70%, 80%, 90% or 100% lower number of colony forming units, than an untreated cell infected with the M. tb. An “effective amount” of the formulation of the disclosure relates the amount of the formulation that when administered to a subject in need thereof, reduces the incidence of or prevents infection of the subject by an infectious agent (including M. tb).

A “therapeutically effective amount” or “an effective amount” of the formulation of the disclosure is an amount of a niclosamide formulation sufficient to treat or prevent infection by a bacterium. In some embodiments, the subject receiving a “therapeutically effective amount” or “an effective amount” of the formulation of the disclosure is exposed to a bacterium but is not infected by the bacterium (does not present signs or symptoms of infection). In some embodiments, the subject receiving a “therapeutically effective amount” or “an effective amount” of the formulation of the disclosure has been exposed to a bacterium or infected by a bacterium, but, following administration of the therapeutically effective amount of the formulation, demonstrates a reduction in a severity of a sign or symptom of the infection (including a reduction in colony forming units following a culture of the infection site from before and after administration of the formulation).

The subject can be a male subject or a female subject.

The “effective amount” of the formulation can be administered topically, intravaginally, intramuscularly or intrarectally.

Every document cited herein, including any cross referenced or related patent or application, is hereby incorporated herein by reference in its entirety unless expressly excluded or limited. The citation of any document is not an admission that it is prior art with respect to any invention disclosed or claimed herein or that it alone, or in any combination with any some reference or references, teaches, suggests or discloses any such invention. Further, to the extent that any meaning or definition of a term in this document conflicts with any meaning or definition of the same term in a document incorporated by reference, the meaning or definition assigned to that term in this document shall govern. As used herein, the term “about,” unless indicated otherwise, refers to the recited value, e.g., amount, dose, temperature, time, percentage, etc., ±10%, ±9%, ±8%, ±7%, ±6%, ±5%, ±4%, ±3%, ±2%, or ±1%.

While particular embodiments of the disclosure have been illustrated and described, various some changes and modifications can be made without departing from the spirit and scope of the disclosure. The scope of the appended claims includes all such changes and modifications that are within the scope of this disclosure.

### EXAMPLES

In order that the invention disclosed herein may be more efficiently understood, examples are provided below. It should be understood that these examples are for illustrative purposes only and are not to be construed as limiting the invention in any manner.

### Example 1

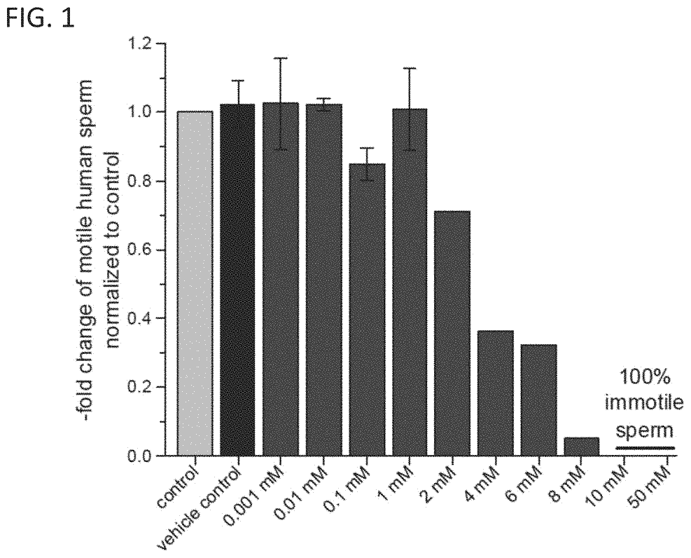

To determine the optimal concentration or dose of niclosamide that completely inhibits the motility of sperm cells in a semen sample (100% inhibition), fresh semen samples were mixed with the formulation in gel form of the disclosure, for 2 minutes. After 2 minutes of incubation with the formulation, the semen samples were collected and analyzed for sperm motility. The fold change in total number of motile sperm cells treated with each concentration of niclosamide, relative to sperm motility normalized to the total number of motile sperm in a control sample (un-treated sample). As for effective concentrations, this in vitro data suggests that the effective niclosamide concentration that causes 100% fold decrease in number of motile sperm cells in a semen sample as compared to a control sample, is 10 mM or higher. Attached is a graph in FIG. 1 that shows the dose-dependent effect of niclosamide on human sperm motility. Data were collected within 2 minutes after mixing the formulation in gel form, with fresh semen, so the effects are instantaneous. As for timing, the gel may be applied 15-20 minutes prior to intercourse to allow gelling to fully occur. Furthermore, the gel may not be efficacious after residing in the vagina for longer than 1 hour.

### Example 2

The objective of the study described herein was to develop a vaginal gel formulation of niclosamide for local administration, which is non-sticky, non-irritating with optimum rheological properties that enables easy extrusion from the dosage form and enables easy application and spreading. As the drug is intended for local action, formulation was designed to ensure minimal absorption into systemic circulation. A dense network of blood vessels is present in vagina making it an excellent route of administration for local drug delivery. Physico-chemical properties such as molecular weight, lipophilicity, molecule solubility, ionization and surface charges etc. may have an impact on drug availability at the site of action. Hence, these properties were considered while designing the vaginal gel formulation for optimum clinical effectiveness. The water solubility of niclosamide has been reported to be about 13.32 μg/mL in its anhydrous form, but this falls to about 0.61 or 0.96 μg/mL for its monohydrate forms (Alhalaweh, A et al., Mol. Pharm. 2014, 11, 3123-3132; Sanphui, Petal., Cryst. Growth Des. 2012, 12, 4588-4599; van Tonder, E. C et al., Int. J. Pharm. 2004, 269, 417-432). The poor solubility of niclosamide in water and the need to disperse niclosamide within a gel to make a homogenous composition, are some of the major challenges for developing a gel formulation. The study described herein, discloses the development of a niclosamide gel formulation comprising excipients that enable both the solubility of therapeutically effective amounts of niclosamide in the gel formulation, as well as release of a therapeutically effective of the niclosamide from the formulation. Several, solvent/cosolvent systems were screened to identify specific solvents or solvent combinations for. The study described herein, provides details of the screening done to select the most suitable excipients (solvent/cosolvent systems, gelling agents, viscosity modulators, buffers/pH modulator and preservatives) and their optimal amounts/concentrations for use in the formulations of the disclosure, based on the compatibility with niclosamide and effect on gelling capacity.

Forced Degradation Data of Drug Substance:

Stress test of niclosamide in neutral, acid, alkaline conditions, forced by temperature effects, oxidation by UV on the dry substance was evaluated by Olon SPA (DMF). The following table details the forced degradation data.

TABLE 3
Summary of forced degradation data
of drug substance (niclosamide).

| S. No. | Conditions | % Degradation |
|---|---|---|
| 1 | No stress Conditions | Impurity 3-0.04% |
| 2 | UV Exposure for 7 days | No degradation |
| 3 | Temperature Effect (100° C. for 8 hours) | No degradation |
| 4 | Neutral Hydrolysis (3 mL H₂O at | Impurity 1-0.02% |
| | 100° C. for 8 hours) | Impurity 2-0.03% |
| 5 | Acid Hydrolysis (3 mL HCl at 100° C. | No degradation |
| | for 8 hours) | |
| 6 | Alkaline Hydrolysis (0.1N NaOH at | Impurity 1 & 2 are |
| | 100° C. for 2 hours) | formed and potency |
| | | decreased to 97% |
| 7 | Oxidative Hydrolysis (3 mL H₂O₂ at | No degradation |
| | 100° C. for 8 hours) | |

Conclusion: Based on forced degradation data it was concluded that the drug substance is prone to alkaline hydrolysis under increased temperature conditions.

Preformulation Studies.

Solubility studies of drug substance: Solubility of niclosamide was conducted in various aqueous buffers and additionally solubility was conducted using different grades of Polyethylene glycol (PEG).

Saturation Solubility in Buffers:

Solubility was established using 100 mL of each of the chosen buffers, and was conducted using shake flask method. To the chosen buffer, drug substance was added in increments of small amounts until the solution showed resistance to solubilize. Further, the solution was agitated at 37° C. for 24 hours and checked physically for un-dissolved mass. This saturated solution was centrifuged and injected an aliquot in to the chromatograph.

TABLE 4
Results of saturation solubility of niclosamide in various Buffers

| S. No. | Buffer/Solvent | Solubility (mg/mL) |
|---|---|---|
| 1 | pH 1.2 (0.1N HCl) | Nil |
| 2 | pH 2.0 (0.01N HCl) | Nil |
| 3 | pH 3.0 acid phthalate buffer | Nil |
| 4 | pH 4.0 acid phthalate buffer | Nil |
| 5 | pH 4.5 acetate buffer | Nil |
| 6 | pH 5.1 acetate buffer | Nil |
| 7 | water^{§} | 0.005 |

^{§}Solubility assessed at room temperature (~25° C.).

Solubility in Solvents/Co-Solvents:

Solubility of the drug was determined in selected solvent/co-solvents. About 10 g of the solvent was taken and small amount of drug was added in increments till 500 mg. This study was carried out to check if 5% w/w of the drug solubility could be achieved. These solutions were further diluted quantitatively to about test concentration and analyzed using chromatograph.

TABLE 5
Solubility data of niclosamide in solvents

| | | Solubility |
|---|---|---|
| S. No. | Solvent | (mg/g) |
| 1 | polyethylene glycol (PEG 400) | 47.4 |
| 2 | polyethylene glycol (PEG 1000) | 46.6 |
| 3 | polyethylene glycol (PEG 2000) | 45.6 |

Saturation Solubility in Various Solvents:

Saturation solubility of niclosamide was studied in the solvents, to know the highest concentration of niclosamide that can be solubilized, as depicted in table 6.

TABLE 6
Saturation solubility of niclosamide in solvents

| Quantity | Quantity of | | | |
|---|---|---|---|---|
| of Solvent | API | Procedure | Remarks | Inference |
| 10 g of | *800 mg | 800 mg of API was added gradually | No clear solution, | 700 mg was |
| PEG-400 | | and solubilised by stirring and | few API particles | solubilised |
| | | simultaneous heating at 50° C. for 40 | were observed. | completely. |
| | | min. | | |
| 10 g of | *600 mg | 600 mg of API was added gradually | No clear solution, | 500 mg was |
| PEG-1000 | | and solubilised by continuous stirring | few API particles | solubilised |
| | | and heating at 50° C. for 40 min. | were observed. | completely. |
| 10 g of | *600 mg | 600 mg of API was added gradually | No clear solution, | 500 mg was |
| PEG-2000 | | and solubilised by continuous stirring | few API particles | solubilised |
| | | and heating at 50° C. for 40 min. | were observed. | completely. |

*500 mg of API was added initially; to it 100 mg was added sequentially after complete solubilization of former 100 mg.

Observation: Based on solubility study data, niclosamide was found to be insoluble in studied buffers like 0.1 N HCl, 0.01 N HCl, pH 3.0 Acid phthalate buffer, pH 4.0 Acid phthalate buffer, pH 4.5 Acetate Buffer or pH 5.1 Acetate Buffer. Heating and stirring improved solubilization of niclosamide in polyethylene glycol. Wherein maximum solubility of 7% w/w in PEG-400 and 5% w/w in PEG-1000 & PEG-2000 was achieved. In further formulation studies, drug substance concentration >7% w/w was desired, so 65° C. of temperature was used for solubilization of API. Hence, a slight impact of heating was observed.

Polymer Screening Studies

Described herein is a study done to shortlist from among several polymers with bio adhesive properties including: polyacrylates (polycarbophil, carbomer, thiolated polyacrylates), cellulosic derivatives (sodium carboxymethylcellulose, hydroxypropylmethylcellulose, hydroxyethylcellulose), based on the gelling capacity in solvents. Polymers used for this study were mixed in the solvents to see their gel formation. All the solutions were kept for 15 hrs at room temperature and observations were made regarding the formation of gel. Table 7 describes the details of solvents with their concentrations, used and it also summarizes the formation of gels.

TABLE 7
Study plan and observations for polymer screening studies.

| | | Polymer | |
|---|---|---|---|
| | | Concentration | |
| Sr. No. | Samples | (% w/w) | Remarks |
| 1 | PEG-400 (5 g) + HEC [Natrosol | 0.5 | Dispersed, no gel formation |
| | 250HHX Pharm] (25 mg) | | |
| 2 | PEG-400 (5 g) + sodium CMC | 0.5 | Dispersed, no gel formation |
| | [Blanose CMC 7HF PH] (25 mg) | | |
| 3 | PEG-400 (5 g) + carbopol 980 NF | 0.5 | Dispersed |
| | (25 mg) | | |
| 4 | PEG-400 (5 g) + HPC [Klucel HF] | 0.5 | Dispersed, no gel formation |
| | (25 mg) | | |
| 5 | PEG-400 (5 g) + pemulen TR-1 NF | 0.5 | Dispersed |
| | (25 mg) | | |
| 6 | PEG-400 (5 g) + polycarbophil | 0.5 | Dispersed |
| | [Noveon AA-1] (25 mg) | | |
| 7 | PEG-400 (5 g) + HPMC K4M | 0.5 | Dispersed |
| | [Methocel K4M] (25 mg) | | |
| 8 | PEG-400 (5 g) + HEC [Natrosol | 0.25 | Dispersed |
| | 250HHX Pharm] (25 mg) + water (5 g) | | |
| 9 | PEG-400 (5 g) + sodium CMC | 0.25 | Dispersed |
| | [Blanose CMC 7HF PH] (25 mg) + | | |
| | water (5 g) | | |
| 10 | PEG-400 (5 g) + carbopol 980 NF | 0.25 | Less viscous gel formed |
| | (25 mg) + water (5 g) | | |
| 11 | PEG-400 (5 g) + HPC [Klucel HF] | 0.25 | Dispersed |
| | (25 mg) + water (5 g) | | |
| 12 | PEG-400 (5 g) + pemulen TR-1 NF | 0.25 | Less viscous gel formed |
| | (25 mg) + water (5 g) | | |
| 13 | PEG-400 (5 g) + polycarbophil | 0.25 | Less viscous gel formed |
| | [Noveon AA-1] (25 mg) + water (5 g) | | |
| 14 | PEG-400 (5 g) + HPMC K4M | 0.25 | Dispersed |
| | [Methocel K4M] (25 mg) + water (5 g) | | |
| 15 | water (20 g) + HEC [Natrosol | 1.0 | Very less viscous gel formed |
| | 250HHX Pharm] (200 mg) | | |
| 16 | water (20 g) + sodium CMC [Blanose | 1.0 | Viscous gel formed |
| | CMC 7HF PH] (200 mg) | | |
| 17 | water (20 g) + carbopol 980 NF | 1.0 | Viscous gel formed |
| | (200 mg) | | |
| 18 | water (20 g) + HPC [Klucel HF] | 1.0 | Very less viscous gel formed |
| | (200 mg) | | |
| 19 | water (20 g) + pemulen TR-1 NF | 1.0 | Viscous gel formed |
| | (200 mg) | | |
| 20 | water (20 g) + polycarbophil [Noveon | 1.0 | Viscous gel formed |
| | AA-1] (200 mg) | | |
| 21 | water (20 g) + HPMC K4M [Methocel | 1.0 | Very less viscous gel formed |
| | K4M] (200 mg) | | |

Observations: Polymers like carbopol, pemulen TR 1, polycarbophil, and HPMC were observed to form gels in PEG-400 alone, and polymers like carbopol, pemulen TR 1, polycarbophil were observed to form gels in mixture of PEG-400 and water.

Drug—excipient compatibility studies: The drug excipient compatibility studies were performed to check the compatibility between the niclosamide API and the proposed inactive ingredients to be used in the formulation. Proposed drug to excipient ratio was selected based on the function of the excipient and maximum concentration to be used. Drug and excipient binary mixtures were prepared by uniform mixing. The blends were packed in clear glass vials and the compatibility was evaluated in closed conditions. In the closed-vial study, the dry mix samples were filled in vials with screw stopper fitting covered with parafilm and stored at 40° C./75% RH, 25° C./60% RH and 2° C.-8° C. storage condition up to 4 weeks. Samples at 40° C./75% RH and 25° C./60% RH were checked for physical description and analyzed for related substances, initially, at 2 weeks and at 4 weeks. Ratio of drug substance to excipients in blend for the compatibility study, are as follows: a) niclosamide:gelling agents/mucoadhesive agents was 1:15; b) niclosamide:solvents/co-solvents was 1:5; c) niclosamide:pH adjusting agents/neutralizing agents was 1:1; and d) niclosamide: preservatives/anti-oxidants was 1:0.5. Summary of physical description and related substances analysis of binary mixtures of API & excipients at initial and 40° C./75% RH-4 weeks condition are presented below in Table 8.

TABLE 8
Summary of drug-excipient compatibility study data (physical description and related substances).

| | | | | | | | | | | | | |
|---|---|---|---|---|---|---|---|---|---|---|---|---|
| | | | | | | | | | | | Single | |
| | | | | | | | | | | | Max | |
| | Sample | Combination/ | Stability | Time | | | | | | | Unk | Total |
| Sr. No. | Code | Mixture | Condition | Interval | Description | Imp-1 | Imp-2 | Imp-3 | Imp-4 | Imp-5 | Imp | Impurities |

Active Pharmaceutical Ingredient

| | | | | | | | | | | | |
|---|---|---|---|---|---|---|---|---|---|---|---|
| 1 | A | niclosamide | Initial | Pale yellow | ND | ND | 0.029 | 0.009 | ND | 0.010 | 0.05 |
| | | | | color | | | | | | (RRT | |
| | | | | solid | | | | | | 0.768) | |

| | | | | | | | | | |
|---|---|---|---|---|---|---|---|---|---|
| 40° C./ | 4 | pale yellow | ND | ND | 0.026 | 0.012 | 0.014 | 0.007 | 0.06 |
| 75% RH | Weeks | color | | | | | | (RRT | |
| | | solid | | | | | | 0.768) | |

Gelling Agents/Mucoadhesive Agents

| | | | | | | | | | | | |
|---|---|---|---|---|---|---|---|---|---|---|---|
| 2 | B | niclosamide + | Initial | White color | 0.008 | ND | 0.036 | 0.018 | ND | ND | 0.06 |
| | | carbopol 980 | | solid | | | | | | | |

| | | | | | | | | | |
|---|---|---|---|---|---|---|---|---|---|
| 40° C./ | 4 | White color | ND | ND | 0.027 | 0.018 | 0.008 | 0.028 | 0.10 |
| 75% RH | Weeks | solid | | | | | | (RRT | |
| | | | | | | | | 0.126) | |

| | | | | | | | | | | | |
|---|---|---|---|---|---|---|---|---|---|---|---|
| 3 | C | niclosamide + | Initial | light yellow | ND | ND | 0.026 | ND | ND | 0.023 | 0.05 |
| | | hydroxypropyl | | color | | | | | | (RRT | |
| | | cellulose | | solid | | | | | | 0.113) | |

| | | | | | | | | | | |
|---|---|---|---|---|---|---|---|---|---|---|
| (Klucel HF | 40° C./ | 4 | Pale yellow | ND | ND | 0.030 | 0.012 | 0.009 | 0.010 | 0.06 |
| Pharm) | 75% RH | Weeks | color | | | | | | (RRT | |
| | | | solid | | | | | | 0.770) | |

| | | | | | | | | | | | |
|---|---|---|---|---|---|---|---|---|---|---|---|
| 4 | D | niclosamide + | Initial | White color | 0.014 | ND | 0.031 | 0.012 | ND | 0.032 | 0.10 |
| | | polycarbophils | | solid | | | | | | (RRT | |
| | | (Noveon AA-1) | | | | | | | | 0.106) | |

| | | | | | | | | | |
|---|---|---|---|---|---|---|---|---|---|
| 40° C./ | 4 | off White | ND | ND | 0.029 | 0.017 | 0.009 | 0.007 | 0.07 |
| 75% RH | Weeks | color | | | | | | (RRT | |
| | | solid | | | | | | 0.768) | |

| | | | | | | | | | | | |
|---|---|---|---|---|---|---|---|---|---|---|---|
| 5 | E | niclosamide + | Initial | off-White | 0.004 | ND | 0.034 | 0.013 | ND | 0.006 | 0.06 |
| | | hydroxypropyl | | color | | | | | | (RRT | |
| | | methylcellulose | | solid | | | | | | 0.142) | |

| | | | | | | | | | | |
|---|---|---|---|---|---|---|---|---|---|---|
| (Methocel | 40° C./ | 4 | Pale yellow | ND | ND | 0.027 | 0.011 | 0.009 | 0.007 | 0.05 |
| K4M) | 75% RH | Weeks | color | | | | | | (RRT | |
| | | | solid | | | | | | 0.767) | |

| | | | | | | | | | | | |
|---|---|---|---|---|---|---|---|---|---|---|---|
| 6 | F | niclosamide + | Initial | pale yellow | 0.008 | ND | 0.028 | 0.012 | ND | 0.005 | 0.05 |
| | | hydroxyethyl | | color | | | | | | (RRT | |
| | | cellulose | | solid | | | | | | 0.164) | |

| | | | | | | | | | | |
|---|---|---|---|---|---|---|---|---|---|---|
| (Natrosol | 40° C./ | 4 | Yellow color | ND | ND | 0.027 | 0.008 | 0.010 | 0.008 | 0.05 |
| 250HHX | 75% RH | Weeks | solid | | | | | | (RRT | |
| Pharm) | | | | | | | | | 0.767) | |

| | | | | | | | | | | | |
|---|---|---|---|---|---|---|---|---|---|---|---|
| 7 | G | niclosamide + | Initial | off-White | ND | ND | 0.025 | 0.015 | ND | 0.054 | 0.10 |
| | | sodium | | color | | | | | | (RRT | |
| | | carboxymethyl | | solid | | | | | | 0.112) | |

| | | | | | | | | | | |
|---|---|---|---|---|---|---|---|---|---|---|
| cellulose | 40° C./ | 4 | light pink | ND | 0.019 | 0.019 | 0.006 | 0.008 | 0.027 | 0.09 |
| (Blanose CMC | 75% RH | Weeks | color | | | | | | (RRT | |
| 7HF PH) | | | solid | | | | | | 0.623) | |

| | | | | | | | | | | | |
|---|---|---|---|---|---|---|---|---|---|---|---|
| 8 | H | niclosamide + | Initial | off-White | ND | 0.004 | 0.038 | 0.005 | ND | 0.013 | 0.07 |
| | | pemulen TR-1 | | color | | | | | | (RRT | |
| | | | | solid | | | | | | 0.126) | |

| | | | | | | | | | |
|---|---|---|---|---|---|---|---|---|---|
| 40° C./ | 4 | off-White | ND | ND | 0.028 | 0.015 | 0.007 | 0.008 | 0.07 |
| 75% RH | Weeks | color | | | | | | (RRT | |
| | | solid | | | | | | 0.768) | |

| | | | | | | | | | | | |
|---|---|---|---|---|---|---|---|---|---|---|---|
| 9 | I | niclosamide + | Initial | pale yellow | ND | ND | 0.034 | ND | ND | 0.006 | 0.04 |
| | | poloxamer | | color | | | | | | (RRT | |
| | | (Kolliphor | | solid | | | | | | 0.112) | |

| | | | | | | | | | | |
|---|---|---|---|---|---|---|---|---|---|---|
| P188) | 40° C./ | 4 | Pale yellow | ND | ND | 0.026 | 0.011 | 0.006 | 0.008 | 0.05 |
| | 75% RH | Weeks | color | | | | | | (RRT | |
| | | | solid | | | | | | 0.768) | |

Solvents/Co-solvents

| | | | | | | | | | | | |
|---|---|---|---|---|---|---|---|---|---|---|---|
| 10 | J | niclosamide + | Initial | pale yellow | 0.002 | ND | 0.030 | 0.012 | 0.009 | 0.006 | 0.06 |
| | | propylene | | color | | | | | | (RRT | |
| | | glycol | | liquid | | | | | | 0.761) | |

| | | | | | | | | | |
|---|---|---|---|---|---|---|---|---|---|
| 40° C./ | 4 | pale yellow | ND | ND | 0.027 | 0.011 | 0.010 | 0.007 | 0.06 |
| 75% RH | Weeks | color | | | | | | (RRT | |
| | | liquid | | | | | | 0.767) | |

| | | | | | | | | | | | |
|---|---|---|---|---|---|---|---|---|---|---|---|
| 11 | K | niclosamide + | Initial | pale yellow | 0.002 | ND | 0.028 | 0.012 | 0.007 | 0.006 | 0.06 |
| | | polyethylene | | color | | | | | | (RRT | |
| | | glycol 1000 | | solid | | | | | | 0.761) | |

| | | | | | | | | | |
|---|---|---|---|---|---|---|---|---|---|
| 40° C./ | 4 | pale yellow | ND | ND | 0.0026 | 0.013 | 0.008 | 0.007 | 0.05 |
| 75% RH | Weeks | color | | | | | | (RRT | |
| | | semi solid | | | | | | 0.767) | |

| | | | | | | | | | | | |
|---|---|---|---|---|---|---|---|---|---|---|---|
| 12 | L | niclosamide + | Initial | pale yellow | ND | ND | 0.028 | 0.010 | 0.006 | 0.010 | 0.06 |
| | | polyethylene | | color | | | | | | (RRT | |
| | | glycol 400 | | liquid | | | | | | 0.139) | |

| | | | | | | | | | |
|---|---|---|---|---|---|---|---|---|---|
| 40° C./ | 4 | pale yellow | ND | ND | 0.027 | 0.011 | 0.009 | 0.007 | 0.05 |
| 75% RH | Weeks | color | | | | | | (RRT | |
| | | semi solid | | | | | | 0.766) | |

| | | | | | | | | | | | |
|---|---|---|---|---|---|---|---|---|---|---|---|
| 13 | M | niclosamide + | Initial | Off-White | ND | ND | 0.026 | 0.013 | 0.010 | 0.031 | 0.08 |
| | | glycerin | | color | | | | | | (RRT | |
| | | | | semi solid | | | | | | 0.139) | |

| | | | | | | | | | |
|---|---|---|---|---|---|---|---|---|---|
| 40° C./ | 4 | Off-White | ND | ND | 0.026 | 0.012 | 0.010 | 0.008 | 0.06 |
| 75% RH | Weeks | color | | | | | | (RRT | |
| | | semi solid | | | | | | 0.768) | |

pH Adjusting Agents/Neutralizing Agents

| | | | | | | | | | | | |
|---|---|---|---|---|---|---|---|---|---|---|---|
| 14 | N | niclosamide + | Initial | pale yellow | ND | ND | 0.029 | 0.011 | 0.011 | 0.013 | 0.10 |
| | | citric acid | | color | | | | | | (RRT | |
| | | | | solid | | | | | | 0.139) | |

| | | | | | | | | | |
|---|---|---|---|---|---|---|---|---|---|
| 40° C./ | 4 | pale yellow | ND | ND | 0.027 | 0.012 | 0.009 | 0.007 | 0.06 |
| 75% RH | Weeks | color | | | | | | (RRT | |
| | | solid | | | | | | 0.765) | |

| | | | | | | | | | | | |
|---|---|---|---|---|---|---|---|---|---|---|---|
| 15 | O | niclosamide + | Initial | pale yellow | ND | ND | 0.026 | 0.010 | 0.010 | 0.010 | 0.06 |
| | | lactic acid | | color | | | | | | (RRT | |
| | | | | solid | | | | | | 0.139) | |

| | | | | | | | | | |
|---|---|---|---|---|---|---|---|---|---|
| 40° C./ | 4 | pale yellow | ND | ND | 0.027 | 0.012 | 0.010 | 0.007 | 0.06 |
| 75% RH | Weeks | color | | | | | | (RRT | |
| | | solid | | | | | | 0.766) | |

| | | | | | | | | | | | |
|---|---|---|---|---|---|---|---|---|---|---|---|
| 16 | P | niclosamide + | Initial | pale yellow | ND | ND | 0.030 | 0.011 | ND | 0.014 | 0.06 |
| | | sorbic acid | | color | | | | | | (RRT | |
| | | | | solid | | | | | | 0.768) | |

| | | | | | | | | | |
|---|---|---|---|---|---|---|---|---|---|
| 40° C./ | 4 | pale yellow | ND | ND | 0.027 | 0.013 | 0.009 | 0.006 | 0.06 |
| 75% RH | Weeks | color | | | | | | (RRT | |
| | | solid | | | | | | 0.766) | |

| | | | | | | | | | | | |
|---|---|---|---|---|---|---|---|---|---|---|---|
| 17 | Q | niclosamide + | Initial | Pale pink | ND | 0.018 | 0.013 | 0.012 | 0.008 | 0.027 | 0.09 |
| | | sodium | | color | | | | | | (RRT | |
| | | hydroxide | | solid | | | | | | 0.622) | |

| | | | | | | | | | |
|---|---|---|---|---|---|---|---|---|---|
| 40° C./ | 4 | Pale pink | 0.294 | 0.243 | 0.013 | 0.012 | 0.009 | 0.024 | 0.62 |
| 75% RH | Weeks | color | | | | | | (RRT | |
| | | solid | | | | | | 0.624) | |

| | | | | | | | | | | | |
|---|---|---|---|---|---|---|---|---|---|---|---|
| 18 | R | niclosamide + | Initial | Orange red | ND | 0.004 | 0.006 | 0.012 | 0.009 | 0.026 | 0.07 |
| | | ethylenediamine | | colored | | | | | | (RRT | |
| | | | | sticky | | | | | | 0.621) | |
| | | | | solid gel | | | | | | | |

| | | | | | | | | | |
|---|---|---|---|---|---|---|---|---|---|
| 40° C./ | 4 | Orange red | 0.003 | ND | 0.028 | 0.013 | 0.008 | 0.011 | 0.08 |
| 75% RH | Week | colored | | | | | | (RRT | |
| | | sticky | | | | | | 0.615) | |
| | | solid gel | | | | | | | |

| | | | | | | | | | | | |
|---|---|---|---|---|---|---|---|---|---|---|---|
| 19 | S | niclosamide + | Initial | Pale yellow | ND | ND | 0.027 | 0.013 | 0.007 | 0.009 | 0.06 |
| | | potassium | | color | | | | | | (RRT | |
| | | bitartrate | | powder | | | | | | 0.768) | |

| | | | | | | | | | |
|---|---|---|---|---|---|---|---|---|---|
| 40° C./ | 4 | Pale yellow | 0.002 | ND | 0.027 | 0.013 | 0.008 | 0.007 | 0.06 |
| 75% RH | Week | color | | | | | | (RRT | |
| | | powder | | | | | | 1.086) | |

Preservatives/Antioxidants

| | | | | | | | | | | | |
|---|---|---|---|---|---|---|---|---|---|---|---|
| 20 | T | niclosamide + | Initial | pale yellow | ND | ND | 0.031 | 0.010 | 0.008 | 0.007 | 0.06 |
| | | benzyl | | color | | | | | | (RRT | |
| | | alcohol | | semi liquid | | | | | | 0.764) | |

| | | | | | | | | | |
|---|---|---|---|---|---|---|---|---|---|
| 40° C./ | 4 | pale yellow | ND | ND | 0.026 | 0.011 | 0.008 | 0.007 | 0.05 |
| 75% RH | Week | color | | | | | | (RRT | |
| | | semi solid | | | | | | 0.761) | |

| | | | | | | | | | | | |
|---|---|---|---|---|---|---|---|---|---|---|---|
| 21 | U | niclosamide + | Initial | Paly-yellow | ND | ND | 0.030 | 0.011 | ND | 0.012 | 0.05 |
| | | benzoic acid | | to off white | | | | | | (RRT | |
| | | | | color | | | | | | 0.163) | |
| | | | | solid | | | | | | | |

| | | | | | | | | | |
|---|---|---|---|---|---|---|---|---|---|
| 40° C./ | 4 | Paly-yellow | ND | ND | 0.026 | 0.001 | 0.008 | 0.007 | 0.05 |
| 75% RH | Week | color | | | | | | (RRT | |
| | | solid | | | | | | 0.767) | |
| 40° C./ | 4 | Pale yellow | 0.002 | ND | 0.028 | 0.014 | 0.008 | 0.007 | 0.06 |
| 75% RH | Week | color | | | | | | (RRT | |
| | | powder | | | | | | 1.086) | |

The impurities detected were as follows: Impurity-1 (5-chloro salicylic acid); Impurity-2 (2-chloro-4-nitro aniline); Impurity-3 (N-(2-chloro-4-nitrophenyl)-2-hydroxybenzamide); Impurity-4 (3,5-dichloro-N-(2-chloro-4-nitrophenyl)-2-hydroxybenzamide); and Impurity-5 (3-chloro-N-(2-chloro-4-nitrophenyl)-2-hydroxybenzamide).

Observations: No significant change in physical description was observed with any of the binary mixtures except with sodium CMC where color was changed from off-white to light pink. However, there was no impact on related substances observed. There was no significant change in related substances with any of the binary mixture even after exposure for 4 weeks except for combination of API with sodium hydroxide. In case of binary mixture containing sodium hydroxide the Total impurities increased significantly from 0.09% (Initial) to 0.62% (40° C./75% RH-4 weeks). Impurity-1 & Impurity-2 were increased upon exposure due to hydrolysis which is catalyzed by heat and alkali. Drug to NaOH ratio proposed to be used in the formulation for pH adjustment was very low. Impact of actual concentration in the formulation was monitored using short term stability study and based on the outcome usage of NaOH in the formulation was concluded. Based on the results described herein, all the gelling agents were considered for the formulation trials. Preservatives: benzoic acid and benzyl alcohol, neutralizing agents: lactic acid, citric acid, sodium hydroxide and Solubilizers: PEG 400, were considered for further trials.

Preliminary Formulation Development Trials:

Described herein are initial formulation trials based on the preformulation data described herein, with the same formula with 3% and with increased drug concentration to 5%, as depicted in Table 9. The same formula was used for further trials with differing gelling agents and increased PEG concentration which are described herein.

TABLE 9
Qualitative and quantitative composition of formulation with
3% niclosamide and with 5% niclosamide [Batch Size: 150 g]

| | | 3% w/w niclosamide | | 5% w/w niclosamide | |
|---|---|---|---|---|---|
| Batch No.: | | Concentration | Quantity/ | Concentration | Quantity/ |
| Sr. No. | Ingredients | (% w/w) | Batch (g) | (% w/w) | Batch (g) |
| 1 | niclosamide | 3.00 | 4.50 | 5.00 | 7.50 |
| 2 | polyethylene glycol-400 | 35.00 | 52.50 | 35.00 | 52.50 |
| 3 | hydroxyethyl cellulose | 3.00 | 4.50 | 3.00 | 4.50 |
| | (Natrosol 250 HHX | | | | |
| | pharm) | | | | |
| 4 | potassium sodium | 0.35 | 0.525 | 0.35 | 0.525 |
| | tartrate | | | | |
| 5 | citric acid | 2.00 | 3.00 | 2.00 | 3.00 |
| 6 | lactic acid | 3.50 | 5.25 | 3.50 | 5.25 |
| 7 | benzoic acid | 0.20 | 0.30 | 0.20 | 0.30 |
| 8 | sodium chloride | 0.70 | 1.05 | 0.70 | 1.05 |
| 9 | sodium hydroxide (1N | q.s. | q.s. | q.s. | q.s. |
| | NAOH) | | | | |
| 10 | water | 50.55 | 75.83 | 48.55 | 72.90 |

Observations: Highly viscous, sticky, non-pourable, light yellow color opaque gel was formed.

Formulation trial with increased concentration of PEG-400 from 35% to 65% w/w: niclosamide was shown to have good solubility in PEG-400 (up to 7% w/w-from saturation solubility studies). Thus to solubilize 5% w/w of drug, concentration of PEG-400 was increased to 65% w/w in the formulation. 65% w/w of PEG-400 can be used for Topical formulations.

TABLE 10
Qualitative and quantitative composition of formulation with 5%
w/w of niclosamide and 65% w/w of PEG-400 [Batch Size: 150 g]

| | | 5% w/w niclosamide | |
|---|---|---|---|
| Batch No.: | | Concentration | Quantity/ |
| Sr. No. | Ingredients | (% w/w) | Batch (g) |
| 1 | niclosamide | 5.00 | 7.50 |
| 2 | polyethylene glycol-400 | 65.00 | 97.50 |
| 3 | hydroxyethyl cellulose | 3.00 | 4.50 |
| | (Natrosol 250 HHX Pharm) | | |
| 4 | potassium bitartrate | 0.35 | 0.525 |
| 5 | citric acid | 2.00 | 3.00 |
| 6 | lactic acid | 3.50 | 5.25 |
| 7 | benzoic acid | 0.20 | 0.30 |
| 8 | sodium chloride | 0.70 | 1.05 |
| 9 | sodium hydroxide (1N NaOH) | q.s. | q.s. |
| 10 | water | 18.55 | 27.90 |

Observations: Highly viscous, non-pourable light yellow color opaque gel was formed.

Formulation Trial with Different Gelling Agents.

Formulation trials were done with various gelling agents with same formula and manufacturing process as used for testing the 5% w/w niclosamide formulation with 65% PEG-400, as described herein.

TABLE 11
Qualitative and quantitative composition of formulations with 5% w/w niclosamide,
65% w/w PEG-400 with different gelling agents (Batch Size: 100 g)

| | | 5% w/w niclosamide with | | 5% w/w niclosamide with | | 5% w/w niclosamide with | | 5% w/w niclosamide with | |
|---|---|---|---|---|---|---|---|---|---|
| | | sodium CMC | | carbopol 980 | | pemulen TR-1 | | polycarbophil | |
| Formulation Types | | Concentration | Quantity/ | Concentration | Quantity/ | Concentration | Quantity/ | Concentration | Quantity/ |
| Sr. No. | Ingredients | (% w/w) | Batch (g) | (% w/w) | Batch (g) | (% w/w) | Batch (g) | (% w/w) | Batch (g) |
| 1 | niclosamide | 5.00 | 5.00 | 5.00 | 5.00 | 5.00 | 5.00 | 5.00 | 5.00 |
| 2 | polyethylene | 65.00 | 65.00 | 65.00 | 65.00 | 65.00 | 65.00 | 65.00 | 65.00 |
| | glycol-400 | | | | | | | | |
| 3 | sodium | 3.00 | 3.00 | — | — | — | — | — | — |
| | carboxymethyl | | | | | | | | |
| | cellulose | | | | | | | | |
| | (Blanose CMC | | | | | | | | |
| | 7HF PH) | | | | | | | | |
| 4 | carbopol 980 | — | — | 0.5 | 0.5 | — | — | — | — |
| 5 | pemulen TR-1 | — | — | — | — | 0.5 | 0.5 | — | — |
| 6 | polycarbophil | — | — | — | — | — | — | 0.5 | 0.5 |
| | (Noveon AA-1) | | | | | | | | |
| 4 | potassium | 0.35 | 0.35 | 0.35 | 0.35 | 0.35 | 0.35 | 0.35 | 0.35 |
| | bitartrate | | | | | | | | |
| 5 | citric acid | 2.00 | 2.00 | 2.00 | 2.00 | 2.00 | 2.00 | 2.00 | 2.00 |
| 6 | lactic acid | 3.50 | 3.50 | 3.50 | 3.50 | 3.50 | 3.50 | 3.50 | 3.50 |
| 7 | benzoic acid | 0.20 | 0.20 | 0.20 | 0.20 | 0.20 | 0.20 | 0.20 | 0.20 |
| 8 | sodium chloride | 0.70 | 0.70 | 0.70 | 0.70 | 0.70 | 0.70 | 0.70 | 0.70 |
| 9 | sodium | q.s. | q.s. | q.s. | q.s. | q.s. | q.s. | q.s. | q.s. |
| | hydroxide (1N | | | | | | | | |
| | NaOH) | | | | | | | | |
| 10 | water | 19.80 | 19.80 | 21.10 | 21.10 | 21.10 | 21.10 | 21.10 | 21.10 |

Observations: Viscous, slightly pourable light yellow color opaque gel was formed.

Physico-chemical evaluation of preliminary formulation trials of niclosamide gels: Preliminary formulation trials with different gelling agents were evaluated for description, assay & related substances as shown in Table 12.

TABLE 12
Physical and chemical evaluation of preliminary formulation trials of niclosamide gel

| | YCT | YCT | | | | | |
|---|---|---|---|---|---|---|---|
| | recipe | recipe | Increased | | | | |
| | with 3% | with 5% | PEG-400 | sodium | carbopol | pemulen | |
| Tests | API | API | Concentration | CMC | 980 | TR-1 | polycarbophil |
| Description | Highly viscous, non-pourable light | | | Viscous, slightly pourable light yellow color gel | | | |
| | yellow color gel formed | | | formed | | | |
| Assay | Not | Not | Not | 101.2 | 96.2 | 95.6 | 95.2 |
| (% w/w) | Done | Done | Done | | | | |
| pH | 3.90 | 3.80 | 4.65 | 4.31 | 3.84 | 4.06 | 3.77 |

Related Substances (% w/w)

| | | | | | | | |
|---|---|---|---|---|---|---|---|
| Impurity-1 | ND | ND | ND | ND | ND | ND | 0.022 |
| Impurity-2 | ND | ND | ND | ND | ND | ND | ND |
| Impurity-3 | 0.028 | 0.035 | 0.028 | 0.026 | 0.027 | 0.027 | 0.027 |
| Impurity-4 | 0.012 | 0.010 | 0.013 | 0.011 | 0.013 | 0.014 | 0.012 |
| Impurity-5 | 0.010 | ND | 0.009 | 0.008 | 0.009 | 0.010 | 0.008 |
| Single | 0.008 | 0.013 | 0.007 | 0.007 | 0.006 | 0.006 | 0.006 |
| Maximum | (RRT- | (RRT- | (RRT- | (RRT- | (RRT- | (RRT- | (RRT- |
| Unknown | 0.764) | 0.766) | 0.139) | 0.766) | 0.766) | 0.767) | 0.766) |
| Total | 0.06 | 0.06 | 0.06 | 0.05 | 0.06 | 0.06 | 0.08 |
| Impurities | | | | | | | |

Observations: Based on above data, it was concluded that there is no significant impact of various gelling agents on related substances of drug product.

Reproducible Formulation Trials with Various Gelling Agents:

Based on the physical observation of the preliminary formulation development trials described herein, few gelling agents were selected, Preservative benzoic acid was replaced with benzyl alcohol due to solubility issues of benzoic acid and reproducible formulation trials with different gelling agents were manufactured, packed in multilaminated tubes, submitted for initial evaluation and loaded for stability at conditions 40° C./75% RH (2W & 4W), 25° C./60% RH (2W & 4W), 60° C. (2W). Formulation composition details, physical and chemical evaluation are reported in Table 13.

TABLE 13
Qualitative and quantitative composition of reproducible
formulation trials of niclosamide gel

| | Batch Size: 250 g | | | | |
|---|---|---|---|---|---|
| | Quantity/Batch ( g) | | | | |
| | Batch No. | | | | |
| | | | | 5% w/w | |
| | 3% w/w | 5% w/w | 5% w/w | API & | 5% w/w |
| | API & | API & | API & | 0.5% w/w | API & |
| | 3% w/w | 3% w/w | 0.5% w/w | pemulen | 3% w/w |
| Ingredients | HEC | HEC | carbopol | TR-1 | polycarbophil |
| niclosamide | 7.50 | 12.50 | 12.50 | 12.50 | 12.50 |
| polyethylene glycol-400 | 162.50 | 162.50 | 162.50 | 162.50 | 162.50 |
| hydroxyethyl cellulose | 7.50 | 7.50 | — | — | — |
| (Natrosol 250 HHX Pharm) | | | | | |
| carbopol 980 | — | — | 1.250 | — | — |
| pemulen TR-1 | — | — | — | 1.250 | — |
| polycarbophil (Noveon AA-1) | — | — | — | — | 1.250 |
| potassium bitartrate | 0.875 | 0.875 | 0.875 | 0.875 | 0.875 |
| citric acid | 5.00 | 5.00 | 5.00 | 5.00 | 5.00 |
| lactic acid | 8.750 | 8.750 | 8.750 | 8.750 | 8.750 |
| benzyl alcohol | 0.50 | 0.50 | 0.50 | 0.50 | 0.50 |
| sodium chloride | 1.75 | 1.75 | 1.75 | 1.75 | 1.75 |
| sodium hydroxide (1N NaOH) | 0.250 | 0.250 | 0.250 | 0.250 | 0.250 |
| water | 55.375 | 50.375 | 56.625 | 56.625 | 56.625 |
| Total | 250.00 | 250.00 | 250.00 | 250.00 | 250.00 |

TABLE 14
Physical and chemical evaluation of preliminary reproducible formulation trials of niclosamide gel-initial formulations

| | | | | | Related Substances (%) | | | | | | |
|---|---|---|---|---|---|---|---|---|---|---|---|
| | | | | | | | | | | Maximum | |
| Tests | | | Viscosity | Assay | Imp- | Imp- | Imp- | Imp- | Imp- | Unknown | Total |
| Formulations | Description* | pH | (cPs) | (% w/w) | 1 | 2 | 3 | 4 | 5 | Impurity | Impurities |
| 3% w/w API & | Complies | 3.70 | — | 97.5 | ND | ND | 0.029 | 0.009 | 0.007 | 0.012 | 0.071 |
| 3% w/w HEC | | | | | | | | | | (RRT | |
| | | | | | | | | | | 0.257) | |
| NCM-5-F-009 | Complies | 3.65 | 700 | 97.5 | 0.003 | ND | 0.030 | 0.014 | 0.009 | ND | 0.056 |
| (5% w/w API & | | | | | | | | | | | |
| 3% w/w HEC) | | | | | | | | | | | |
| NCM-5-F-010 | Complies | 3.68 | 11000 | 104.1 | ND | ND | 0.027 | 0.009 | ND | 0.004 | 0.040 |
| (5% w/w API & | | | | | | | | | | (RRT | |
| 0.5% w/w | | | | | | | | | | 1.066) | |
| carbopol) | | | | | | | | | | | |
| NCM-5-F-011 | Complies | 3.69 | 58000 | 102.4 | 0.002 | ND | 0.030 | 0.015 | 0.010 | 0.006 | 0.068 |
| (5% w/w API & | | | | | | | | | | (RRT | |
| 0.5% w/w | | | | | | | | | | 0.761) | |
| pemulen TR-1) | | | | | | | | | | | |
| NCM-5-F-012 | Complies | 3.71 | 18000 | 97.1 | 0.030 | ND | ND | 0.015 | 0.009 | 0.006 | 0.060 |
| (5% w/w API & | | | | | | | | | | (RRT | |
| 3% w/w | | | | | | | | | | 0.764) | |
| polycarbophil) | | | | | | | | | | | |

*Pale yellow colored homogeneous opaque gel;
ND—Not Detected

Observations: Viscosity data was found to be in increasing order as: HEC<carbopol<polycarbophil<pemulen base gels. Gel with HEC (700 cPs) as gelling agent had lowest viscosity and with pemulen TR-1 (58000 cPs) had highest viscosity. Based on above data, it was concluded that there is no significant impact of various gelling agents on pH and related substances of drug product.

Stability studies summary: Reproducible batches of niclosamide gel formulations described herein, were prepared and charged for stability at the accelerated (40±2° C. and 75±5% RH), long term (25±2° C. and 60±5% RH), and 60° C. The summary of data for the samples that were withdrawn is presented in Table 15. Product was observed to show satisfactory stability for all the batches studied in the multilaminated packs at stress studies and accelerated condition.

TABLE 15
Summary of stability study results of niclosamide gel formulations.

| | | | | Related Substances (%) | | | | | | |
|---|---|---|---|---|---|---|---|---|---|---|
| Tests | | | | | | | | | Max. | |
| | Testing | | | Imp- | Imp- | Imp- | Imp- | Imp- | Unk. | Total |
| Condition | Period | Description* | pH | 1 | 2 | 3 | 4 | 5 | Impurity | Impurities |

Formulation: 3% API & 3% HEC

| Initial | | Complies | 3.70 | ND | ND | 0.029 | 0.009 | 0.007 | 0.012 | 0.071 |
|---|---|---|---|---|---|---|---|---|---|---|
| | | | | | | | | | (RRT | |
| | | | | | | | | | 0.257) | |
| 60° C. | 1 | Complies | — | 0.004 | 0.003 | 0.027 | 0.017 | ND | 0.006 | 0.073 |
| | Week | | | | | | | | (RRT | |
| | | | | | | | | | 1.054) | |
| | 2 | Complies | 3.68 | 0.008 | 0.004 | 0.029 | 0.013 | 0.006 | 0.009 | 0.092 |
| | Week | | | | | | | | (RRT | |
| | | | | | | | | | 0.575) | |
| 40° C./ | 2 | Complies | — | 0.002 | ND | 0.028 | 0.013 | 0.008 | 0.008 | 0.063 |
| 75% RH | Week | | | | | | | | (RRT | |
| | | | | | | | | | 1.087) | |
| | 4 | Complies | 3.54 | 0.004 | 0.002 | 0.028 | 0.009 | 0.007 | 0.004 | 0.054 |
| | Week | | | | | | | | (RRT | |
| | | | | | | | | | 0.773) | |

Formulation: 5% API & 3% HEC

| Initial | | Complies | 3.65 | 0.003 | ND | 0.030 | 0.014 | 0.009 | ND | 0.056 |
|---|---|---|---|---|---|---|---|---|---|---|
| 60° C. | 1 | Complies | — | 0.004 | 0.004 | 0.030 | 0.014 | ND | 0.003 | 0.055 |
| | Week | | | | | | | | (RRT | |
| | | | | | | | | | 0.284) | |
| | 2 | Complies | 3.68 | 0.009 | 0.004 | 0.030 | 0.014 | 0.008 | 0.007 | 0.093 |
| | Week | | | | | | | | (RRT | |
| | | | | | | | | | 0.768) | |
| 40° C./ | 2 | Complies | — | 0.004 | ND | 0.028 | 0.013 | 0.008 | 0.005 | 0.062 |
| 75% RH | Week | | | | | | | | (RRT | |
| | | | | | | | | | 1.085) | |
| | 4 | Complies | 3.61 | 0.006 | 0.002 | 0.027 | 0.010 | 0.008 | 0.004 | 0.057 |
| | Week | | | | | | | | (RRT | |
| | | | | | | | | | 0.772) | |

Formulation: 5% API & 0.5% carbopol-980

| Initial | | Complies | 3.68 | ND | ND | 0.027 | 0.009 | ND | 0.004 | 0.040 |
|---|---|---|---|---|---|---|---|---|---|---|
| | | | | | | | | | (RRT | |
| | | | | | | | | | 1.066) | |
| 60° C. | 1 | Complies | — | 0.004 | ND | 0.029 | 0.011 | ND | 0.019 | 0.083 |
| | Week | | | | | | | | (RRT | |
| | | | | | | | | | 1.055) | |
| | 2 | Complies | 3.90 | 0.010 | 0.003 | 0.029 | 0.020 | 0.008 | 0.019 | 0.131 |
| | Week | | | | | | | | (RRT | |
| | | | | | | | | | 0.575) | |
| 40° C./ | 2 | Complies | — | 0.003 | ND | 0.028 | 0.012 | 0.008 | 0.005 | 0.059 |
| 75% RH | Week | | | | | | | | (RRT | |
| | | | | | | | | | 1.087) | |
| | 4 | Complies | 3.27 | 0.004 | 0.001 | 0.028 | 0.010 | 0.012 | 0.005 | 0.062 |
| | Week | | | | | | | | (RRT | |
| | | | | | | | | | 0.772) | |

Formulation: 5% API & 0.5% pemulen TR-1

| Initial | | Complies | 3.69 | 0.002 | ND | 0.030 | 0.015 | 0.010 | 0.006 | 0.068 |
|---|---|---|---|---|---|---|---|---|---|---|
| | | | | | | | | | (RRT | |
| | | | | | | | | | 0.761) | |
| 60° C. | 1 | Complies | — | 0.003 | 0.005 | 0.028 | 0.011 | ND | 0.004 | 0.054 |
| | Week | | | | | | | | (RRT | |
| | | | | | | | | | 1.067) | |
| | 2 | Complies | 3.50 | 0.006 | 0.004 | 0.028 | 0.016 | 0.007 | 0.007 | 0.077 |
| | Week | | | | | | | | (RRT | |
| | | | | | | | | | 0.768) | |
| 40° C./ | 2 | Complies | — | 0.003 | ND | 0.028 | 0.012 | 0.008 | 0.006 | 0.061 |
| 75% RH | Week | | | | | | | | (RRT | |
| | | | | | | | | | 1.087) | |
| | 4 | Complies | 3.54 | 0.003 | 0.001 | 0.027 | 0.009 | 0.008 | 0.004 | 0.059 |
| | Week | | | | | | | | (RRT | |
| | | | | | | | | | 0.770) | |

Formulation: 5% API & 0.5% polycarbophil

| Initial | | Complies | 3.71 | 0.030 | ND | ND | 0.015 | 0.009 | 0.006 | 0.060 |
|---|---|---|---|---|---|---|---|---|---|---|
| | | | | | | | | | (RRT | |
| | | | | | | | | | 0.764) | |
| 60° C. | 1 | Complies | — | 0.003 | 0.005 | 0.004 | 0.015 | ND | 0.028 | 0.058 |
| | Week | | | | | | | | (RRT | |
| | | | | | | | | | 0.621) | |
| | 2 | Complies | 4.08 | 0.008 | 0.003 | 0.028 | 0.019 | 0.008 | 0.016 | 0.110 |
| | Week | | | | | | | | (RRT | |
| | | | | | | | | | 0.576) | |
| 40° C./ | 2 | Complies | — | 0.003 | ND | 0.028 | 0.012 | 0.008 | 0.006 | 0.060 |
| 75% RH | Week | | | | | | | | (RRT | |
| | | | | | | | | | 1.087) | |
| | 4 | Complies | 3.43 | 0.003 | 0.002 | 0.029 | 0.010 | 0.008 | 0.004 | 0.058 |
| | Week | | | | | | | | (RRT | |
| | | | | | | | | | 0.772) | |

*Pale yellow colored homogeneous opaque gel,
ND—Not Detected,
RRT—Relative Retention Time*

Observations: Related substances data was found to be almost similar at 60° C.-1W & 2W and 40° C./75% RH-2W & 4W when compared with initial data except for formulation batches with carbopol-980 and polycarbophil. In which related substances were increased at 60° C.-2W when compared with initial. Moreover, there was no significant impact on description and pH upon stability when compared with initial data.

Proposed Formula Composition and Process:

Based on the results obtained and inferences drawn from prototype formulation development, a qualitative and quantitative composition and manufacturing process proposed for further optimization and scale-up, as shown below.

TABLE 16
Qualitative and quantitative composition
for niclosamide gel 5% w/w

| | | Quantity |
|---|---|---|
| Name of | Concentration | per |
| Ingredients | (% w/w) | Gram (g) |
| niclosamide | 5.00 | 0.050 |
| polyethylene glycol-400 | 65.00 | 0.650 |
| gelling agent* | 0.50 | 0.005 |
| potassium bitartrate | 0.35 | 0.0035 |
| citric acid | 2.00 | 0.020 |
| lactic acid | 3.50 | 0.035 |
| benzyl alcohol | 0.20 | 0.002 |
| sodium chloride | 0.70 | 0.007 |
| sodium hydroxide | 0.10 | 0.001 |
| purified water | 22.65 | 0.2265 |
| Total | 100 | 1.00 |

*Gelling agents would be selected from carbopol (0.5% w/w), pemulen TR-1 (0.5% w/w), polycarbophil (0.5% w/w), HEC (3% w/w)

Provided herein is a manufacturing/preparation process for the niclosamide formulations disclosed herein, as follows: 1. In the first vessel, niclosamide can be mixed in PEG-400 for 30 minutes at 500-600 RPM using overhead stirrer. It can be further solubilised with simultaneous heating and stirring for 30 min. benzoic acid and benzyl alcohol can be added to the solution and solubilised by stirring; 2. In a separate vessel 20 g water, sodium chloride, citric acid, lactic acid, potassium tartrate and carbopol 980 can be combined and mixed for 15 minutes at 100-200 RPM using overhead stirrer; 3. Aqueous fraction of step 3 can be added to the PEG fraction of step 2 under stirring and mixing vigorously for 15 minutes at 900-1200 RPM using overhead stirrer; 4. 1N NaOH can be added to adjust the pH to ˜3.70. (˜2.5 mL and for pH found to be 3.80 of ˜3.70); 5. Remaining water (7.90 g) can be added to get the final volume; 6. Homogeneity can be achieved by mixing for 10 minutes at 500-600 RPM using overhead stirrer.

### Example 3

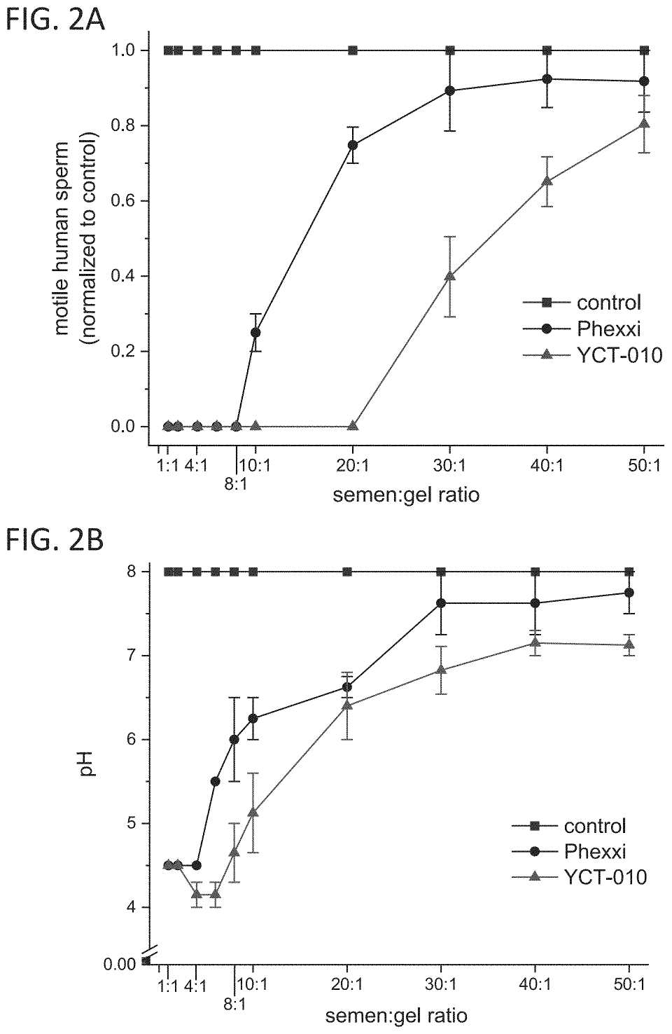

Described herein is a study comparing the efficacy of the niclosamide formulation disclosed herein with Phexxi, a commercially available contraceptive gel from Evofemin, in inhibiting sperm motility and in pH buffering capacity. Fresh human semen was mixed with niclosamide gel formulation of the disclosure, at different ratios, and human sperm motility and pH were measured. Results described herein, show that the niclosamide gel formulation of the disclosure, maintained complete inhibition of sperm cells in human semen at semen to gel ratio of 20:1. In contrast, incubation of human semen with Phexxi resulted in motile sperm cells at a semen to semen to gel ratio of 10:1 (FIG. 2A). Further, as depicted in FIG. 2B, the pH buffering capacity (ability to maintain pH between 3-5), of the niclosamide gel formulation of the present disclosure is significantly higher than that of Phexxi, at the same semen to gel ratio (4:1 to 10:1). Based on the results described herein, the niclosamide gel formulation of the present disclosure, is more effective in inhibiting sperm motility and pH buffering capacity, as compared to commercially available contraceptive formulations.

### Example 4

Described herein is a study to determine drug release over time from the niclosamide gel formulation, as disclosed herein. Two initial niclosamide gel formulations (Formulation A and Formulation B) described in Table 17 below, were covered with vaginal fluid simulant (VFS) and incubated at 37° C. At time points t=0, 5, 10, 30 and 60 minutes, VFS was aspirated and analyzed by LC-MS/MS to detect niclosamide.

TABLE 17
Drug and excipient concentrations of niclosamide gel
formulations A and B, used in drug release study.

| | % w/w | grams |
|---|---|---|
| Formulation A | | |
| NaCl | 0.795731136 | 0.085 |
| lactic acid | 9.361542782 | 1 |
| water | 65.53079948 | 7 |
| 1N NaOH | 18.72308556 | 2 |
| HEC | 2.527616551 | 0.27 |
| NA | 3.06122449 | 0.327 |
| Total | 100 | 10.682 |
| Formulation B | | |
| NaCl | 0.79573 | 0.085 |
| lactic acid | 9.36154 | 1 |
| water | 56.1693 | 6 |
| 1N NaOH | 18.7231 | 2 |
| HEC | 2.52762 | 0.27 |
| PEG-400 | 9.36154 | 1 |
| NA | 3.06122 | 0.327 |
| Total | 100 | 10.682 |

TABLE 18
Drug release from niclosamide gel formulations A and B.

| | niclosamide | |
|---|---|---|
| time | released (mM) | |
| (minutes) | Formulation A | Formulation B |

| | | | |
|---|---|---|---|
| 0 | min | 0.039 | <LOD |
| 5 | min | 0.034 | <LOD |
| 10 | min | 0.057 | <LOD |
| 30 | min | <LOD | <LOD |
| 60 | min | 2.2 | 7.6 |

<LOD; Below the limit of detection

Conclusion: Data from the study shows niclosamide release from the gel formulations of the disclosure.

### Example 5

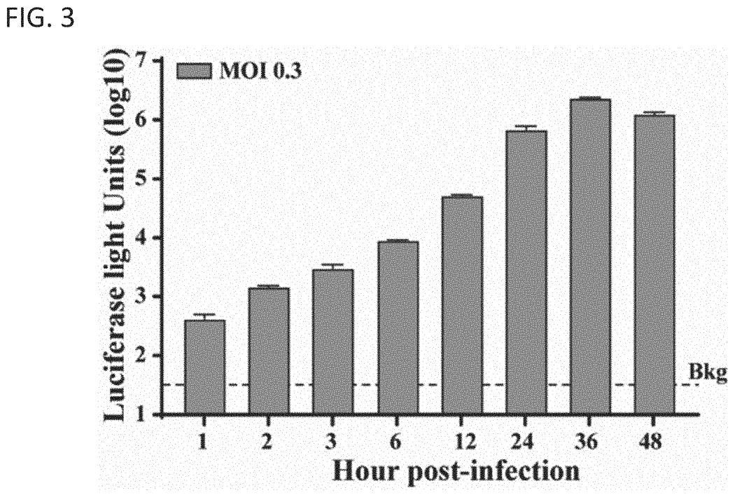

To facilitate high-throughput antiviral testing, a nano luciferase gene was engineered into the open-reading-frame 7 (ORF7) of the SARS-CoV-2 genome. When cells were infected with such reporter virus, the cells expressed luciferase that could be quantified to indicate viral replication. As shown in FIG. 3, increasing amounts of luciferase signals were detected in Vero E6 cells after infected with the reporter virus from 1 to 36 h post-infection. At 36 h post-infection, viral infection-induced cytopathic effect was observed, leading to a decrease of luciferase signal at 48 h post-infection. The results suggest that luciferase signal could be used as a surrogate to test antiviral inhibitors.

### Example 6

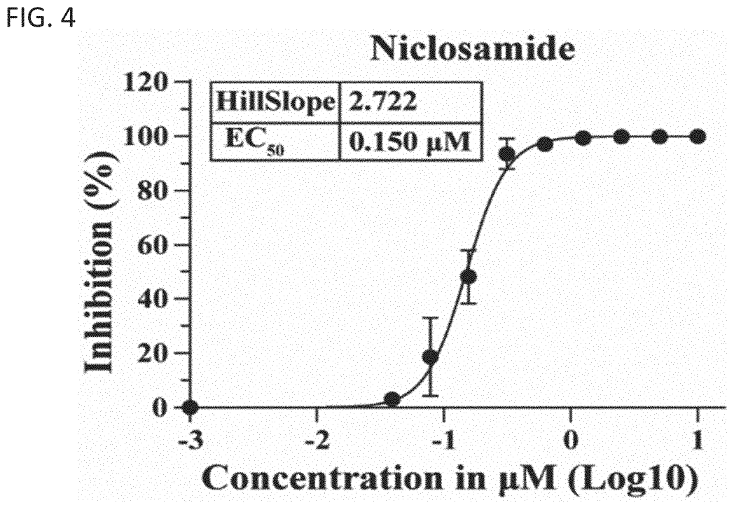

Described herein is a study determining the activity of niclosamide (an antiparasitic agent that has broad spectrum of antiviral activity) against SARS-CoV-2. niclosamide powder was dissolved in 100% DMSO at 10 mM. The antiviral assay was performed in a 96-well plate. Approximately 1.5×10⁴ Vero E6 cells were seeded to each well the night before viral infection. Different concentrations of niclosamide were added to cells together with reporter SARS-CoV-2 (MOI of 0.1). At each tested condition, the cell medium contained a final concentration of 0.5% DMSO. After 24 h post-infection, the infected cells were assayed for luciferase activity using Nano-Glo Luciferase System following the manufacture's protocol (Promega). The EC₅₀ value was derived from the luciferase curve (FIG. 4). niclosamide showed a potent antiviral EC₅₀ of 150 nM.

### EQUIVALENTS

Those skilled in the art will recognize or be able to ascertain using no more than routine experimentation, many equivalents to the specific embodiments of the invention described herein. Such equivalents are intended to be encompassed by the following claims.

All references cited herein are incorporated herein by reference in their entirety and for all purposes to the same extent as if each individual publication or patent or patent application was specifically and individually indicated to be incorporated by reference in its entirety for all purposes.

The present invention is not to be limited in scope by the specific embodiments described herein. Indeed, various modifications of the invention in addition to those described herein will become apparent to those skilled in the art from the foregoing description and accompanying figures. Such modifications are intended to fall within the scope of the appended claims.

Unless indicated otherwise, all percentages by are percentages by weight, parts are parts by weight, temperature is in degrees Celsius, and pressure is at or near atmospheric. There are numerous variations and combinations of reaction conditions, e.g., component concentrations, desired solvents, solvent mixtures, temperatures, pressures and other reaction ranges and conditions that can be used to optimize the product purity and yield obtained from the described process. Only reasonable and routine experimentation will be required to optimize such process conditions.

1. A method of treating or preventing an infection by a virus in a subject having or at risk for a viral infection and/or inhibiting or preventing viral replication of a virus in a subject having or at risk for a viral infection, comprising administering to a subject an effective amount of a formulation comprising an amount of niclosamide and one or more of a humectant, a lubricant, a solvent, an osmolality modulator, a pH modulator, a viscosity modulator and a preservative,
wherein the formulation inhibits viral replication of the virus, optionally thereby treating or preventing a viral infection.

2. The method of claim 1, wherein the virus is a Herpes-Simplex virus (HSV), Human immunodeficiency virus (HIV), Coronavirus, Severe acute respiratory syndrome coronavirus (SARS-CoV-1), Severe acute respiratory syndrome coronavirus (SARS-CoV-2), Human coronavirus-Subtype OC43 (HCoV-OC43), Middle eastern respiratory syndrome corona virus (MERS-CoV), Vesicular stomatitis virus—Zaire Ebola virus (VZVEBOV), Dengue virus (DENV), Powassan virus (POWV), Enterovirus (EV), Influenza A virus (FluAV), Human metapneumovirus (HMPV), Rabies virus (RABV), Rift valley fever virus (RVFV), Zika virus (ZIKV), West-nile virus (WNV), Yellow fever virus (YFV), Sindbis virus (SINV), Junin virus (JUNV), Lassa mammarenavirus (LASV), or Lymphocytic choriomeningitis virus (LCMV).

3-4. (canceled)

5. The method of claim 1, wherein the administering step comprises administering the formulation by a local route to achieve a concentration of at least about 150 nM in the subject, optionally administering by intramuscular injection.

6. (canceled)

7. The method of claim 1, wherein the administration step results in a concentration of niclosamide at a site of administration of the formulation in the subject, of about 100 nM to about 1 μM, about 100 nM to about 500 nM, about 100 nM to about 250 nM, about 100 nM to about 150 nM, or 150 nM.

8-11. (canceled)

12. The method of claim 1, wherein the formulation is administered locally, optionally administered topically, intrarectally, intramuscularly or intravaginally.

13. (canceled)

14. The method of claim 1, wherein the subject is human.

15. The method of claim 1, wherein the formulation has a viscosity of between about 700 cPs and about 58,000 cPs, between about 10,000 cPs and about 18,000 cPs, or about 11,000 cPs, optionally wherein the viscosity modulator is HEC, carbopol, polycarbophil, or pemulen.

16-20. (canceled)

21. The method of claim 1, wherein the humectant, the lubricant or the solvent comprises a synthetic polymer, optionally the humectant, the lubricant or the solvent comprises at least two monomers of polyethylene glycol (PEG), optionally PEG-400, PEG-1000, or PEG-2000.

22. (canceled)

23. The method of claim 21, wherein the humectant, the lubricant or the solvent comprises PEG-400.

24. The method of claim 1, wherein the method comprises an amount of PEG-400 between 0.1% and 75%, between 35% and 75%, between 35% and 65%, about 65%, or 65% of the total weight of the formulation, inclusive of the endpoints.

25-28. (canceled)

29. The method of claim 1, wherein the formulation is a semisolid form, a gel, or a cream.

30. (canceled)

31. The method of claim 1, wherein the amount of niclosamide is between 0.1% and 10%, between 3% and 7%, between 3% and 6%, between 3% and 5%, about 5%, or 5% of total weight of the formulation, inclusive of the endpoints.

32-36. (canceled)

37. The method of claim 1, wherein the niclosamide has a concentration of between 4 mM and 500 mM, between 10 mM and 250 mM, about 150 mM, or 150 mM, inclusive of the endpoints.

38-42. (canceled)

43. The method of claim 1, wherein the formulation comprises an amount of the osmolality modulator of less than 5% or less than 1% of the total weight of the formulation, optionally the osmolality modulator comprises sodium chloride.

44-45. (canceled)

46. The method of claim 1, wherein the formulation comprises an amount of the pH modulator of less than 10%, less than 5%, less than 1%, or less than 0.5% of the total weight of the formulation, and wherein the pH modulator comprises one or more of lactic acid, potassium sodium tartrate, citric acid monohydrate, potassium bitartrate, and sodium hydroxide.

47-49. (canceled)

50. The method of claim 1, wherein the viscosity modulator is a viscosity enhancer, optionally the viscosity enhancer comprising one or more of hydroxyethyl cellulose, alginic acid, polycarbophil and carbopol, and optionally the formulation comprises an amount of a viscosity enhancer of between 1% and 10%, between 1% and 5%, less than 5%, 3%, less than 1%, about 0.5%, or 0.5% of the total weight of the formulation, inclusive of the endpoints.

51-62. (canceled)

63. The method of claim 1, wherein the preservative comprises one or more of benzyl alcohol, chlorhexidine gluconate and benzoic acid.

64-65. (canceled)

66. The method of claim 1, wherein the formulation comprises an amount of the preservative of less than 1%, about 0.2%, 0.2%, or less than 0.2% of the total weight of the formulation.

67-69. (canceled)

70. The method of claim 1, wherein the formulation comprises:
a) niclosamide at a concentration of 3% to 5% by weight of the formulation;
b) a humectant, a lubricant or a solvent, wherein the humectant, the lubricant or the solvent comprises an amount of PEG-400 of 35% to 65% by weight of the formulation and an amount of water of 10% to 60% by weight of the formulation;
c) an osmolality modulator, wherein the osmolality modulator comprises an amount of sodium chloride of 0.1% to 1% by weight of the formulation;
d) at least one pH modulator, wherein the at least one pH modulator comprises an amount of lactic acid of 1% to 5% by weight of the formulation, an amount of citric acid monohydrate of 1% to 5% by weight of the formulation, an amount of potassium sodium tartrate of 0.1% to 0.5% by weight of the formulation, and an amount of sodium hydroxide of 0.1% to 0.5% by weight of the formulation;
e) at least one viscosity modulator, wherein the at least one viscosity modulator comprises an amount of carbopol of 0.1% to 1% by weight of the formulation; and
f) at least one preservative, wherein the at least one preservative comprises an amount of benzyl alcohol of 0.1% to 0.5% by weight of the formulation.

71. The method of claim 1, wherein the formulation comprises:
a) niclosamide at a concentration of 100 mM;
b) a humectant, a lubricant or a solvent, wherein the humectant, the lubricant or the solvent comprises an amount of PEG-400 of between 0.1% and 65% by weight of the formulation and an amount of water of between 10% and 90% by weight of the formulation;
c) an osmolality modulator, wherein the osmolality modulator comprises an amount of sodium chloride of less than 1% by weight of the formulation;
d) at least one pH modulator, wherein the at least one pH modulator comprises an amount of lactic acid of less than 10% by weight of the formulation, an amount of citric acid monohydrate of less than 5% by weight of the formulation, an amount of potassium bitartrate of less than 0.5% by weight of the formulation, and an amount of sodium hydroxide of less than 1% by weight of the formulation;
e) at least one viscosity modulator, wherein the at least one viscosity modulator comprises an amount of hydroxyethyl cellulose of 3% by weight of the formulation, an amount of alginic acid of less than 5% by weight of the formulation, an amount of polycarbophil of less than 5% by weight of the formulation and an amount of carbopol of less than 5% by weight of the formulation; and
f) at least one preservative, wherein the at least one preservative comprises an amount of benzyl alcohol of less than 1% by weight of the formulation, an amount of chlorhexidine gluconate of less than 0.12% by weight of the formulation, and an amount of benzoic acid of less than 0.2% by weight of the formulation.

72. The method of claim 1, wherein the formulation comprises niclosamide at a concentration of:
a) between 0.01 μM and 20 μM;
b) between 0.04 μM and 1.1 μM;
c) between 0.01 μM and 0.44 μM;
d) between 0.04 μM and 0.30 μM;
e) between 0.12 μM and 0.10 μM;
f) between 0.3 μM and 5 μM; or
g) between 2.5 μM and 5 μM.

73-74. (canceled)

75. A formulation for use in the method of claim 1.
